(19) United States
(12) Patent Application Publication
(10) Pub. No.: US 2008/0280933 A1
(43) Pub. Date: Nov. 13, 2008

(54) BENZIMIDAZOLYL COMPOUNDS

(76) Inventors: Ivan Efremov, Gales Ferry, CT (US); Bruce N. Rogers, Mystic, CT (US); Allen J. Duplantier, Ledyard, CT (US); Lei Zhang, Waterford, CT (US); Qian Zhang, Levittown, NY (US); Noha S. Maklad, Waterford, CT (US); Edelweiss Evrard, Eastry (GB); Michael A. Brodney, East Lyme, CT (US)

Correspondence Address: PFIZER INC;Steve T. Zelson, 150 EAST 42ND STREET, 5TH FLOOR - STOP 49, NEW YORK, NY, 10017-5612, US

(21) Appl. No.: 11/780,579

(22) Filed: Jul. 20, 2007

Related U.S. Application Data
(60) Provisional application No. 60/833,149, filed on Jul. 25, 2006.

(51) Int. Cl. C07D 401/02; A61K 31/454; A61K 31/438; C07D 487/10; A61P 25/00

(52) U.S. Cl. 514/278; 546/199; 514/322; 546/18

(57) ABSTRACT

Compounds and pharmaceutically acceptable salts of the compounds are disclosed, wherein the compounds have the structure of Formula I

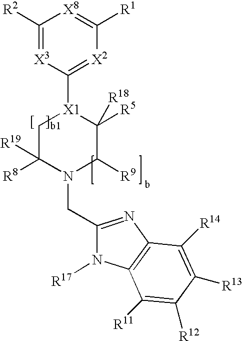

as defined in the specification. Corresponding pharmaceutical compositions, methods of treatment, methods of synthesis, and intermediates are also disclosed.

### CROSS-REFERENCE TO RELATED APPLICATIONS

This application claims priority from U.S. Provisional application Ser. No. 60/833,149 Filed Jul. 25, 2006, which is incorporated by reference in its entirety.

### FIELD OF THE INVENTION

The present invention comprises a novel class of benzimidazolyl compounds having the structure of formula I (including tautomers and salts of those compounds) and pharmaceutical compositions comprising a compound of formula I. The present invention also comprises methods of treating a subject by administering a therapeutically effective amount of a compound of formula I to the subject. These compounds are useful for the conditions disclosed herein. The present invention further comprises methods for making the compounds of formula I and corresponding intermediates.

### BACKGROUND OF THE INVENTION

The present invention provides potentiators of glutamate receptors (compounds of formula I), pharmaceutical compositions thereof, and methods of using the same, processes for preparing the same, and intermediates thereof.

Glutamate is an abundant and important neurotransmitter in mammalian CNS that is involved in a variety of normal CNS functions and has been suggested to be involved in CNS disorders. The functions of glutamate as a neurotransmitter are mediated by two families of glutamate receptors on cells in the CNS—the ionotropic glutamate receptor family, which contain integral ion channels, and the metabotropic glutamate receptor family whose members are linked to G-proteins (Ozawa et al., Prog. Neurobiol., 1998, 54, 581-618). The mGlu receptors are part of the Type III G-protein coupled receptor (GPCR) superfamily, which also includes the GABA-B receptors, calcium-sensing receptor, putative pheromone receptors, and taste receptors (Pin et al., Pharmacol Ther., 2003, 98, 325-354).

A key feature in the understanding of many members of the Type III GPCR superfamily that has emerged recently is the recognition of multiple binding sites on these receptors for different classes of pharmacological agents. One class of agents bind to the extracellular endogenous ligand binding site on the receptor (the orthosteric site)—both pharmacological agonists and antagonists that bind to this site have been described for members of the Type III receptor superfamily (Conn and Pin, Ann. Rev. Pharmacol. Toxicol., 1997, 37, 205-237). More recently, for many receptors in the Type III superfamily (including multiple types of mGlu receptors), compounds have been described that bind to regions of the receptor distinct from the orthosteric site (Pin et al., Mol. Pharmacol., 2001, 60, 881-884). These are termed allosteric ligands, and for many type III receptors the discovery of allosteric ligands has provided pharmacological tools which can be differentiated in chemical structure from orthosteric ligands.

Allosteric compounds may also provide pharmacological distinctions not possible with orthosteric ligands. For example, allosteric compounds may not directly activate a receptor, but rather modulate (by enhancing or reducing) the activity of the endogenous ligand upon its binding to the orthosteric site. In addition, pharmacological distinctions include the potential for pharmacological specificity between related receptors types that share the same endogenous ligand. For example, the structural similarly of the glutamate binding site on closely related members of the mGlu receptor family has resulted in the development of agonist and antagonist compounds that bind to this site which are similar in potency toward multiple receptor within a family. There may be advantages to targeting the development of novel, selective pharmacological agents for these receptors that bind at allosteric sites, since other regions of the receptors show less homology across receptor subtypes than the glutamate binding site.

The metabotropic glutamate (mGlu) receptors include eight subtypes which have been categorized into three groups based on their structural homologies, the second messenger systems to which they are linked, and their pharmacology. The mGlu receptors are found on both CNS neurons and glia, and have been implicated in a variety of CNS functions. Because of the key role of glutamate in CNS function, pharmacological manipulation of this class of glutarnate receptors has been suggested as an avenue to treat a variety of diseases (Conn and Pin, Ann. Rev. Pharmacol. Toxicol., 1997, 37, 205-237; Schoepp and Conn, Trends Pharmacol. Sci., 1993, 14, 13-20).

The present invention relates to the mGluR2 subtype of mGlu receptor, which together with mGluR3 receptors comprise the group II mGlu receptors. mGluR2 receptors have been shown to modulate synaptic transmission at both excitatory glutamate-releasing and inhibitory GABA-releasing neurons (Schoepp, J Pharmacol. Exp. Ther., 2001, 299, 12-20). The pharmacological tools that have been used to probe the functions of mGluR2 receptors are direct agonist and competitive antagonist compounds that have activity at both mGluR2 and mGluR3 receptors. Compounds that bind to allosteric sites of the mGluR2 receptor may allow differentiation from the activities of these orthosteric ligands. Pharmacological manipulations of mGluR2 have been suggested to be useful for a variety of disorders (Marek, Current Opinion in Pharmacology, 2004, 4, 18-22). These include anxiety and related disorders (Tizzano et al., Pharmacol. Biochem., Behav., 2002, 73, 367-374), stress disorders (Eur J. Pharmacol., 2002, 435, 161-170), depression (Feinberg et al., Pharmacol Biochem, Behav., 2002, 73, 467-474), schizophrenia (Klodzinska et al., Pharmacol Biochem, Behav., 2002, 73, 327-332; Moghaddam and Adams, Science, 1998, 281, 1349-1352), pain disorders including chronic pain syndromes (Varney and Gereau, Curr. Drug Target CNS Neurol. Disorders, 2002, 1, 283-296), seizure disorders and epilepsy (Moldrich et al., Neuropharmacol., 2001, 41, 8-18), Parkinson's (Bradley et al., J. Neurosci., 2000, 20, 3085-3094), neurodegenerative disorders and brain injury (Bond et al., J. Pharmacol Exp. Ther., 2000, 294, 800-809; Allen et al., J. Pharmacol Exp. Ther, 1999, 290, 112-290), and substance abuse (Helton et al., Neuropharmacol., 1998, 36, 1511-1516).

Pin et al., European J. Pharmacology 375 (1999), pp. 277-294, describes the role of mGluR2 agonists and antagonists in regulating the activity of many synapses in the central nervous system, thereby affecting a wide number of physiological and pathological processes.

Johnson et al., J. Med. Chem. 2003, 46, 3189-3192, describes mGluR2 potentiators that have antianxiolytic activity.

All journal articles cited hereinabove are incorporated by reference herein in their entirety.

WO 01/56990 states that mGluR2 receptor potentiators may be effective in the treatment of neurological and psychiatric disorders associated with glutamate dysfunction, including: acute neurological and psychiatric disorders such as cerebral deficits subsequent to cardiac bypass surgery and grafting, stroke, cerebra ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain and post-traumatic pain), tardive dyskinesia, sleep disorders (including narcolepsy), attention deficit/hyperactivity disorder, and conduct disorder.

A need still exists for new drug therapies for the treatment of subjects suffering from or susceptible to the above disorders or conditions. In particular, a need still exists for new drugs having one or more improved properties (such as safety profile, efficacy, or physical properties) relative to those currently available.

### SUMMARY OF THE INVENTION

The invention is directed to a class of compounds, including the pharmaceutically acceptable salts of the compounds, having the structure of formula I:

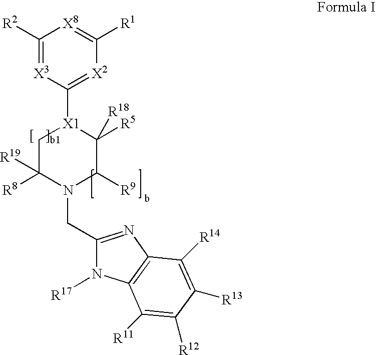

wherein:

X³=CR⁶;

X²=CR⁴;

X⁸=CR³;

R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ wherein each of R¹, R², R³, R⁴ and R⁶ alkyl, alkenyl, cycloalkyl, cycloalkenyl, heterocycloalkyl, aryl or heteroaryl is optionally independently substituted with one or more substituents independently selected from the group consisting of halogen, cyano, R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², NR¹⁰¹C(O)R¹⁰³, and C(O)R¹⁰³;

or two substituents bonded to adjacent carbon atoms of the ring containing X², X³ and X⁸, together with the adjacent carbon atoms, form a heterocyclic or carbocyclic ring which is optionally substituted with one or more R¹⁰, wherein each R¹⁰ is independently selected from the group consisting of hydrogen, —CN, halogen, —C(O)R¹⁰¹, —C(O)NR¹⁰¹R¹⁰², —NR¹⁰¹R¹⁰², —OR¹⁰¹, or —R¹⁰¹;

q is 0, 1 or 2;

each R¹⁰¹ and each R¹⁰² is independently selected from the group consisting of hydrogen, alkyl, alkenyl, alkynyl, cycloalkyl, aryl, heterocycloalkyl and heteroaryl;

wherein each R¹⁰¹ and R¹⁰² alkyl, alkenyl alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

R¹⁰³ is independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

X¹=CR⁷;

b=0, 1 or 2;

b1=1 or 2;

each of R⁵, R⁸ and R⁹ is independently selected from the group consisting of halogen, cyano, —R⁴⁰¹, —OR⁴⁰¹, —C(O)OR⁴⁰¹ and —NR⁴⁰¹R⁴⁰²;

R⁷ is hydrogen, halogen, hydroxyl, alkyl, alkoxy, cyano or alkyl-CO—;

or R⁵ and R⁷ taken together form a second bond;

R¹⁶ is hydrogen, halogen or alkyl;

R¹⁹ is hydrogen or —R⁸ and —R¹⁹ together form ═O;

wherein R⁴⁰¹ and R⁴⁰² are independently selected from the group consisting of hydrogen, alkyl, alkenyl, cycloalkyl, aryl, heterocycloalkyl and heteroaryl;

wherein each of the R⁴⁰¹ and R⁴⁰² alkyl alkenyl, cycloalkyl, aryl, heterocycloalkyl and heteroaryl substituents is optionally independently substituted with one or more substituents independently selected from the group consisting of halogen, hydroxy, cyano, nitro, —R⁴¹¹, —C(O)R⁴¹³, —C(O)OR⁴¹³, —C(O)NR⁴¹¹R⁴¹², —OR⁴¹¹, —OC(O)R⁴¹³, —NR⁴¹¹R⁴¹², —NR⁴¹¹C(O)R⁴¹³, —NR⁴¹¹C(O)OR⁴¹³, —NR⁴¹¹S(O)₂R⁴¹³, —S(O)₁R⁴¹³, —S(O)₂NR⁴¹¹R⁴¹²;

t is 0, 1 or 2;

R⁴¹¹ and R⁴¹² are independently selected from the group consisting of hydrogen, alkyl, cycloalkyl, aryl, heterocycloalkyl and heteroaryl;

R⁴¹³ is independently selected from the group consisting of alkyl, cycloalkyl, aryl, heterocycloalkyl and heteroaryl;

wherein the R⁴¹¹, R⁴¹² and R⁴¹³ alkyl, cycloalkyl, aryl, heterocycloalkyl and heteroaryl substituents are each optionally independently substituted with one or more substituents independently selected from the group consisting of halogen, hydroxy, cyano, nitro, alkyl, aryl, heterocycloalkyl, heteroaryl, haloalkyl, hydroxyalkyl, carboxy, alkoxy and alkoxycarbonyl;

or R⁴ and R⁵ together with the atoms connecting R⁴ and R⁵ form a 5-7-membered carbocyclic or heterocyclic ring optionally containing a heteroatom selected from O, N and S;

or if b=1 and b1=1, R⁵ and R⁹ together with the atoms connecting R⁵ and R⁹ form a 5-7-membered carbocyclic or heterocyclic ring containing up to two heteroatoms selected from O, N and S, wherein the carbocyclic or heterocyclic ring is optionally substituted with one or more substitutents selected from halogen, cyano, alkyl, cycloalkyl, heterocycloalkyl, aryl, heteroaryl or —C(O)R²⁰, wherein R²⁰ is alkyl, cycloalkyl, heterocycloalkyl, aryl or heteroaryl and R²⁰ is optionally substituted with one or more substituents independently selected from the group consisting of alkyl, alkoxy, aryloxy, cyano, —CO₂-alkyl, and —OC(O)alkyl,

or R⁴ and R⁷ together with the atoms connecting R⁶ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms R⁴ and R⁷ contains a heteroatom selected from the group of O, N and S;

or R⁵ and R⁷ together with the atoms connecting R⁵ and R⁷ form a 3-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;

wherein the carbocyclic or heterocyclic ring formed by R⁴ and R⁷ together with the atoms connecting R⁴ and R⁷, or by R⁵ and R⁷ together with the atoms connecting R⁵ and R⁷, is optionally substituted with one or more substitutents independently selected from halogen, cyano, alkyl, cycloalkyl, heterocycloalkyl, aryl, heteroaryl and C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl or —OC(O)alkyl;

R¹⁷ is selected from the group consisting of alkyl, alkenyl, cycloalkyl, and cycloalkenyl, wherein the R¹⁷ alkyl, alkenyl, cycloalkyl, or cycloalkenyl is optionally substituted with one or more substituents independently selected from the group consisting of halogen, hydroxy, cyano, nitro, —R⁵⁰¹, —OR⁵⁰¹, —NR⁵⁰¹R⁵⁰², —S(O)ᵥR⁵⁰³, —S(O)₂NR⁵⁰¹R⁵⁰², —NR⁵⁰¹S(O)₂R⁵⁰³, —OC(O)R⁵⁰³, —C(O)OR⁵⁰³, —C(O)NR⁵⁰¹R⁵⁰², —NR⁵⁰¹C(O)R⁵⁰³, and —C(O)R⁵⁰³;

v is 0, 1 or 2,

wherein each R⁵⁰¹ and each R⁵⁰² is independently selected from the group consisting of hydrogen, alkyl, alkenyl, cycloalkyl, cycloalkenyl, aryl, heterocycloalkyl and heteroaryl;

R¹¹, R¹², R¹³ and R¹⁴ are each independently selected from the group consisting of halogen, cyano, —R⁶⁰¹, —C(O)OR⁶⁰¹, —C(O)NR⁶⁰¹R⁶⁰², —OR⁶⁰¹, —OC(O)R⁶⁰², —NR⁶⁰¹R⁶⁰¹, and —NR⁶⁰¹C(O)R⁶⁰²

wherein R⁶⁰¹ and R⁶⁰² are each independently selected from the group consisting of hydrogen, alkyl, alkenyl, cycloalkyl, aryl, heterocycloalkyl and heteroaryl;

wherein the R⁶⁰¹ and R⁶⁰² alkyl, alkenyl, cycloalkyl, aryl, heterocycloalkyl and heteroaryl substituents are each optionally independently substituted with one or more substituents independently selected from the group consisting of halogen, hydroxy, cyano, nitro, —R⁶¹¹, —C(O)R⁶¹³, —C(O)OR⁶¹³, —C(O)NR⁶¹¹R⁶¹², —OR⁶¹¹, —OC(O)R⁶¹³, —NR⁶¹¹R⁶¹², —NR⁶¹¹C(O)R⁶¹³, —NR⁶¹¹C(O)OR⁶¹³, —NR⁶¹¹S(O)₂R⁶¹³, —S(O)ᵤR⁶¹³, —S(O)₂NR⁶¹¹R⁶¹²;

u is 0, 1 or 2;

R⁶¹¹ and R⁶¹² are each independently selected from the group consisting of hydrogen, alkyl, cycloalkyl, aryl, heterocycloalkyl and heteroaryl;

R⁶¹³ is independently selected from the group consisting of alkyl, cycloalkyl, aryl, heterocycloalkyl and heteroaryl.

In one embodiment of the invention, R¹⁷ is selected from the group consisting of alkyl and cycloalkyl; wherein the R¹⁷ alkyl and cycloalkyl substituents are optionally substituted with one or more substituents independently selected from the group consisting of halogen, cyano, —OR⁵⁰¹, and —NR⁵⁰¹R⁵⁰².

In another embodiment of the invention, at least one of R¹, R², R³, R⁴ and R⁶ is a heterocycloalkyl that contains a nitrogen that is directly bonded to the phenyl ring containing X², X³ and X⁶, wherein the R¹, R², R³, R⁴ or R⁶ heterocycloalkyl is optionally substituted as defined in formula I.

In another embodiment of the invention, at least one of R¹, R², R³, R⁴ and R⁶ is a heteroaryl that contains a nitrogen that is directly bonded to the phenyl ring containing X², X³ and X⁶, wherein the R¹, R², R³, R⁴ or R⁶ heteroaryl is optionally substituted as defined in formula I.

In another embodiment of the invention, at least one of R¹, R², R³, R⁴ or R⁶ is —CO-heterocycloalkyl, wherein the heterocycloalkyl in the —CO-heterocycloalkyl contains a nitrogen that is directly bonded to —CO—, wherein the heterocycloalkyl in the —CO-heterocycloalkyl is optionally substituted as defined in formula I.

In another embodiment of the invention, at least one of R¹, R², R³, R⁴ or R⁶ is —CO-heteroaryl, wherein the heteroaryl in the —CO-heteroaryl contains a nitrogen that is directly bonded to —CO—, wherein the heteroaryl in the —CO-heteroaryl is optionally substituted as defined in formula I.

In another embodiment of the invention, R¹⁰¹ is heterocycloalkyl that contains a nitrogen that is directly bonded to the R¹, R², R³, R⁴ or R⁶ alkyl, alkenyl, cycloalkyl, cycloalkenyl, heterocycloalkyl, aryl, or heteroaryl, wherein the R¹⁰¹ heterocycloalkyl is optionally substituted as defined in formula I.

In another embodiment of the invention, R¹⁰¹ is heteroaryl that contains a nitrogen that is directly bonded to the R¹, R², R³, R⁴ or R⁶ alkyl, alkenyl, cycloalkyl, cycloalkenyl, heterocycloalkyl, aryl or heteroaryl, wherein the R¹⁰¹ heteroaryl is optionally substituted as defined in formula I.

In another embodiment of the invention, —C(O)R¹⁰³ is —CO-heterocycloalkyl, wherein the heterocycloalkyl contains a nitrogen that is directly bonded to CO, wherein the R¹⁰³ heterocycloalkyl in the COR¹⁰³ is optionally substituted as defined in formula I.

In another embodiment of the invention, —C(O)R¹⁰³ is —CO-heteroaryl, wherein the heteroaryl contains a nitrogen that is directly bonded to CO, wherein the R¹⁰³ heteroaryl in the COR¹⁰³ is optionally substituted as defined in formula I.

In another embodiment of the invention, —SO₂R¹⁰³ is —SO₂heterocycloalkyl, wherein the heterocycloalkyl contains a nitrogen that is directly bonded to SO₂, wherein the R¹⁰³ heterocycloalkyl in the SO₂R¹⁰³ is optionally substituted as defined in formula I.

In another embodiment of the invention, —SO₂R¹⁰³ is SO₂heteroaryl, wherein the heteroaryl contains a nitrogen that is directly bonded to SO₂, wherein the R¹⁰³ heteroaryl in the SO₂R¹⁰³ is optionally substituted as defined in formula I.

In another embodiment of the invention, R⁷ is hydrogen, fluoro or alkyl.

In another embodiment of the invention, each of R¹¹, R¹², R¹³ and R¹⁴ is independently selected from the group consisting of hydrogen, halogen, cyano, alkyl, alkoxy, cycloalkyl, aryl, heterocycloalkyl and heteroaryl, wherein the R¹¹, R¹², R¹³ and R¹⁴ alkyl, cycloalkyl, aryl, heterocycloalkyl and heteroaryl substituents are optionally independently substituted as in the compound of formula I.

Preferably, each of R¹¹, R¹², R¹³ and R¹⁴ is independently selected from the group consisting of hydrogen, cyano and halogen.

In another embodiment of the invention, b=1 and b1=0.

In another embodiment of the invention, b=1 and b1=1

In another embodiment of the invention, b and b1 are not both equal to 2.

In another embodiment of the invention, the compound of formula I has the formula II

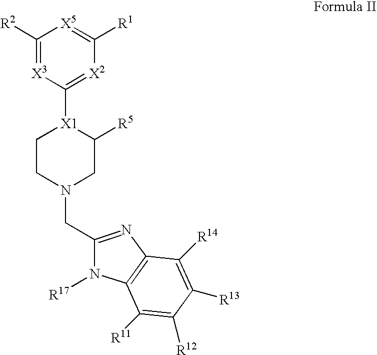

wherein,

R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ or, wherein each of R¹, R², R³, R⁴ and R⁶ alkyl, alkenyl, cycloalkyl, cycloalkenyl, heterocycloalkyl, aryl or heteroaryl is optionally independently substituted with one or more substitutents independently selected from the group consisting of halogen, cyano, —R¹⁰¹, OR¹⁰¹, —NR¹⁰¹R¹⁰², —S(O)_{q}R¹⁰³, —S(O)₂NR¹⁰¹R¹⁰², NR¹⁰¹S(O)₂R¹⁰³, —OC(O)R¹⁰³, —C(O)OR¹⁰³, C(O)NR¹⁰¹R¹⁰², NR¹⁰¹C(O)R¹⁰³, —C(O)R¹⁰³;

R⁵ is selected from the group consisting of halogen, —R⁴⁰¹, —OR⁴⁰¹, and —NR⁴⁰¹R⁴⁰²;

R⁷ is hydrogen, halogen, hydroxyl, alkyl, or alkoxy,

or R⁴ and R⁷ together with the atoms connecting R⁴ and R⁷ form a 5-7-membered carbocyclic or heterocyclic ring, wherein if the ring formed by R⁴ and R⁷ together with the atom connecting R⁴ and R⁷ is a heterocyclic ring, the heterocyclic ring formed by R⁴ and R⁷ together with the atoms connecting R⁴ and R⁷ contains a heteroatom selected from the group of O, N and S;

or R⁵ and R⁷ together with the atoms connecting R⁵ and R⁷ form a 3-7-membered carbocyclic or heterocyclic ring, such as a 5-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;

wherein the carbocyclic or heterocyclic ring formed by R⁴ and R⁷ together with the atoms connecting R⁴ and R⁷, or by R⁵ and R⁷ together with the atoms connecting R⁵ and R⁷, is optionally substituted with one or more substitutents independently selected from halogen, cyano, alkyl, cycloalkyl, heterocycloalkyl, aryl, heteroaryl and —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

In another embodiment of the compound of formula II, R⁷ is hydrogen or fluoro.

In another embodiment of the compound of formula II, R⁵ is hydrogen, halogen or alkyl optionally substituted with one or more fluorines.

In another embodiment of the compound of formula II, R¹⁷ is selected from the group consisting of alkyl and cycloalkyl wherein the R¹⁷ alkyl and cycloalkyl substituent is optionally substituted as in the compound of formula II.

In another embodiment of the compound of formula II, R¹¹, R¹², R¹³ and R¹⁴ are each independently selected from the group consisting of hydrogen, halogen, cyano, alkyl, alkoxy, cycloalkyl, aryl, heterocycloalkyl and heteroaryl, wherein the R¹¹, R¹², R¹³ or R¹⁴ alkyl, cycloalkyl, aryl, heterocycloalkyl and heteroaryl are each optionally independently substituted as in the compound of formula II.

Preferably, each of R¹¹, R¹², R¹³ and R¹⁴ is independently selected from the group consisting of hydrogen, cyano and halogen.

In another embodiment of the invention, the compound of formula II has the formula III,

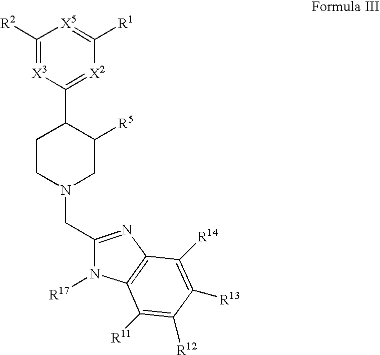

wherein,

R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ or, wherein each of R¹, R², R³, R⁴ and R⁶ alkyl, alkenyl cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, and —C(O)R¹⁰³; and

R⁵ is hydrogen, halogen or alkyl optionally substituted with one or more fluorines.

In one embodiment of Formula III,

R¹⁴ is selected from the group consisting of hydrogen and halogen;

R¹³ is selected from the group consisting of hydrogen, halogen, cyano, alkyl, amino, heterocycloalkyl and heteroaryl;

R¹² is selected from the group consisting of hydrogen, halogen, cyano, alkyl, heterocycloalkyl and heteroaryl, and

R¹¹ is selected from the group consisting of hydrogen, halogen, alkyl, aryl, heterocycloalkyl and heteroaryl.

In another embodiment of Formula III.

R¹¹, R¹², R¹³ and R¹⁴ are each independently selected from the group consisting of alkyl, cycloalkyl, heterocycloalkyl, heteroaryl and aryl and are each optionally independently substituted with one or more substituents independently selected from the group consisting of halogen, alkyl, haloalkyl, alkoxy and alkoxycarbonyl.

In another embodiment of compounds of formula III, R⁵ is hydrogen.

In another embodiment of compounds of formula III, R⁵ is alkyl or alkyl substituted with one or more fluorines.

In another embodiment of compounds of formula III, R⁵ and the aromatic ring containing X², X³ and X⁸ are cis- to each other.

In another embodiment of compounds of Formula III, R¹⁷ is alkyl or cycloakyl, wherein the R¹⁷ alkyl or cycloalkyl substituent is optionally substituted as in the compound of formula III.

In another embodiment of compounds of formula III, R¹⁴ is hydrogen, fluoro or bromo; R¹³ is hydrogen, cyano, halogen, methyl or amino; R¹² is selected from the group consisting of hydrogen, bromo, fluoro, cyano, methyl, methoxy and methoxypyridinyl; and R¹¹ is selected from the group consisting of bromo, fluoro, phenyl and methoxypyridinyl.

In another embodiment of compounds of formula III, R¹⁷ is methyl, cyclopropyl, fluoroethyl, fluoromethyl, methoxyethyl or methoxymethyl.

In another embodiment of the compound of Formula III, R¹⁷ is selected from the group consisting of alkyl and cycloalkyl; R¹⁷ is optionally substituted with one or more substituents independently selected from the group consisting of halogen, alkyl, haloalkyl, alkoxy and alkoxycarbonyl.

In another embodiment of the compound of formula III,

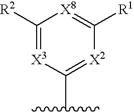

is aryl, optionally substituted as in the compound of formula III.

The aryl is preferably phenyl or naphthalenyl or tetrahydronaphthalenyl, optionally substituted as in the compound of formula III.

In one embodiment where

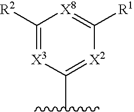

is aryl,

R¹¹, R¹², R¹³ and R¹⁴ are each independently selected from the group consisting of hydrogen, halogen, cyano, alkyl, aryl, haloalkyl, amino, heterocycloalkyl and heteroaryl, wherein the R¹¹, R¹², R¹³ and R¹⁴ alkyl, heterocycloalkyl, aryl or heteroaryl are each optionally independently substituted with one or more substituents independently selected from the group consisting of halogen, alkyl, haloalkyl, alkoxy and alkoxycarbonyl.

In another embodiment of the invention, the compound of formula I has the formula IV,

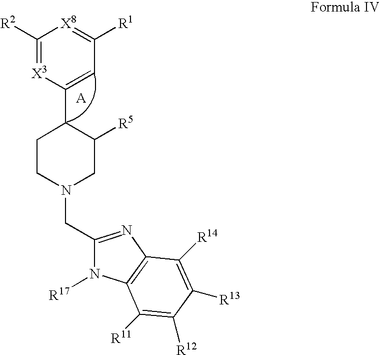

wherein,

X³=CR⁶

X⁸=CR³

R¹, R², R³, and R⁶ are each independently selected from the group consisting of hydrogen, halogen, —CN, —OR¹⁰¹, alkyl alkenyl, cycloalkyl, cycloalkenyl, heterocycloalkylaryl, heteroaryl, —C(O)R¹⁰¹, —C(O)NR¹⁰¹R¹⁰², —NR¹⁰¹R¹⁰², or, wherein each of R¹, R², R³, and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O) R¹⁰³, —C(O)OR¹⁰³, —C(O)NR¹⁰¹R¹⁰², —NR¹⁰¹C(O)R¹⁰³, and —C(O)R¹⁰³

R⁵ is hydrogen, halogen or alkyl optionally substituted with one or more fluorines; and

wherein ring A is a 5-7-membered carbocyclic or heterocyclic ring, wherein A is optionally substituted with one or more substitutents independently selected from halogen, cyano; alkyl optionally substituted with heterocycloalkyl; cycloalkyl, heterocycloalkyl, aryl, heteroaryl, —C(O)OR²⁰ or —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

In an embodiment of compounds of formula IV, R⁵ is alkyl or alkyl substituted with one or more fluorines.

In an exemplary embodiment, the compound of formula IV is a compound of formula IVa:

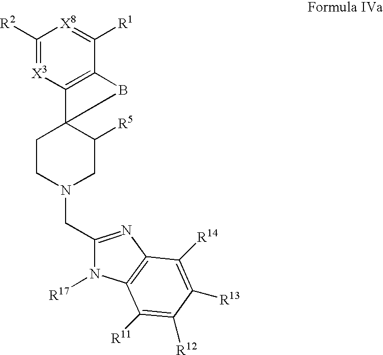

wherein B is a divalent chain selected from the group consisting of ethylene, ethynelene, propylene, butylene, methylenoxy, methylenethioxy, methylenamino, ethylenoxy, ethylenethioxy, and ethylenamino.

wherein the carbons or the N of the methylenamino or ethylenamino divalent chain and the carbons of the ethylene, ethynelene, propylene, butylene, metheylenoxy, ethylenoxy, methylenethioxy, and ethylenethioxy divalent chain are each optionally independently substituted with one or more substitutent is independently selected from halogen, cyano; alkyl optionally substituted with heterocycloalkyl; cycloalkyl, heterocycloalkyl, aryl, heteroaryl or —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂alkyl, or —OC(O)alkyl. The foregoing embodiment is intended to include compounds of formula IVa wherein a heteroatom of the divalent chain B is bonded to the carbon of the piperidine ring as well as compounds of formula IVa wherein a heteroatom of the divalent chain B is bonded to the carbon of the ring containing X³ and X⁸. In one exemplary embodiment, the N of the methylenamino or ethylenamino is optionally substituted with one or more substitutents independently selected from halogen, cyano, alkyl, cycloalkyl, heterocycloalkyl, aryl, heteroaryl or C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, CO₂-alkyl, or OC(O)alkyl.

In an exemplary embodiment of the invention,

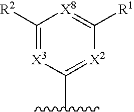

is selected from the group consisting of the following substituents:

4-fluoro-2-methoxyphenyl, 5-fluoro-2-methoxy phenyl, 5-chloro-2-methoxyphenyl, 5-chloro-2-ethoxyphenyl, 5-chloro-2-propoxyphenyl, 5-chloro-2-isobutoxyphenyl, isobutoxyphenyl, butoxyphenyl, 5-chloro-2-((S)-2-methyl-butoxy)-phenyl, 5-Chloro-2-((R)-2-methyl-butoxy)-phenyl, 2-butoxy-5-chlorophenyl, 5-Chloro-2-(tetrahydro-pyran-2-ylmethoxy)phenyl, 5-Chloro-2-(3-methyl-oxetan-3-ylmethoxy)-phenyl, 5-Chloro-2-(tetrahydro-furan-2-ylmethoxy)-phenyl, 5-Chloro-2-(tetrahydro-furan-3-ylmethoxy)-phenyl, 5-Chloro-2-(2-methyl-cyclopropylmethoxy)-phenyl, 5-Chloro-2-(2-cyclopropyl-ethoxy)-phenyl, 5-Chloro-2-cyclobutylmethoxy-phenyl, cyclobutylmethoxy-phenyl, 4-fluoro-3-methoxyphenyl, 2-fluoro-6-methoxyphenyl, difluorophenyl, chlorofluorophenyl, chlorophenyl, bromophenyl, dibromophenyl, fluorophenyl, 2-methoxy-4-trifluoromethylphenyl, trifluoromethylphenyl, [dimethylmorpholin-4-yl]methylphenyl, (2-morpholin-4-yl-ethoxy)-phenyl, methylphenyl, dimethylphenyl, 4-chloro-3-trifluoromethylphenyl, methoxyphenyl, dimethoxyphenyl, hydroxyphenyl, phenyl, fluorophenyl, cyclopentylaminocarbonylphenyl, [N-cyclopropylmethyl]propylaminocarbonylphenyl, [methylpyridynyl]aminocarbonylphenyl, fluorochromanyl, ethylphenyl, t-butylphenyl, cyanophenyl, trifluoromethoxyphenyl, isopropoxyphenyl, 2-methoxy-4-trifluoromethylphenyl, 2-methoxy-5-trifluoromethylphenyl, 2-fluoro-5-trifluoromethylphenyl, 2-fluoro-4-trifluoromethylphenyl, bis-trifluoromethylphenyl, hydroxyethylphenyl, 4-fluoro-2-methylphenyl, 5-Chloro-2-prop-2-ynyloxy-phenyl, prop-2-ynyloxy-phenyl, naphthalenyl, aminocarbonylnaphthalenyl, (1-phenyl-ethoxy)-phenyl, (Indan-2-yloxy)-phenyl, [(S)-(tetrahydro-furan-3-yl)oxy]-phenyl, (tetrahydro-pyran-4-yloxy)-phenyl, ((S)-1-methyl-pyrrolidin-2-ylmethoxy)-phenyl, (2-pyridin-2-yl-ethoxy)-phenyl, ((S)-2-methyl-butoxy)-phenyl, cyclopropyl-ethoxyphenyl, pentoxyphenyl, 3-ethoxypropoxyphenyl, 2-ethoxyethoxyphenyl, 2-isopropoxyethoxyphenyl, 3-dimethylaminopropoxyphenyl, cyclopentylmethoxyphenyl, 2-(2,6-Dimethyl-morpholin-4-yl)-ethoxy)-phenyl, (2,6-Dimethyl-morpholin-4-yl)-phenyl, methoxycarbonylphenyl, methylsulfonyamidophenyl, methyl-cyclopropylmethoxyphenyl, propynyloxyphenyl, 5-chloro-2-propynyloxyphenyl, 5-chloro-2-(3-tetrahydrofuranyl)methoxyphenyl, 5-chloro-2-(3-tetrahydropyranyl)methoxyphenyl, 5-chloro-2-(2-tetrahydrofuranyl)methoxyphenyl, 5-chloro-2-2-tetrahydropyranyl)methoxyphenyl, ethoxyphenyl, N-(5-methyl-1H-pyrazol-3-yl)aminocarbonylphenyl, 3-fluoro-4-trifluoromethyl-phenyl, 2-fluoro-4-trifluoromethoxyphenyl, 2-methyl-4-trifluoromethoxyphenyl, 4-chloro-2-methylphenyl, 4-fluoro-2-methylphenyl, 2-chloro-4-trifluoromethylphenyl, 2-chloro-4-isopropoxyphenyl, 2-fluoro-4-isopropoxy phenyl, 3-fluoro-4-isopropoxyphenyl, 3-chloro-4-isopropoxyphenyl, 3-chloro-4-ethoxyphenyl, 4-methoxy-2-trifluoromethylphenyl, difluoromethoxyphenyl, 2-fluoro-4-difluoromethoxyphenyl, 2-chloro-4-difluoromethoxyphenyl, trifluorophenyl, tetralinyl, 4-fluoro-2-isopropoxyphenyl, 4-fluoro-3-trifluoromethylphenyl, (2,3-dihydro-1-benzofuran-5-yl), 4-fluoro-2-trifluoromethylphenyl, 4-chloro-2-trifluoromethylphenyl, 2-chloro-4-methylphenyl, 3-chloro-4-trifluoromethoxyphenyl, 2-chloro-4-trifluoromethoxy-phenyl, 2-methoxy-4-trifluoromethoxyphenyl, 2-trifluoromethyl-4-isopropoxyphenyl, 2-fluoro-6-trifluoromethylphenyl, dichlorophenyl, 3-chloro-4-trifluoromethylphenyl, 2-methyl-4-trifluoromethylphenyl, 3-methyl-4-trifluoromethylphenyl, 4-fluoro-2-difluoromethoxyphenyl, 3-methoxy-4-trifluoromethylphenyl. In the previous substituents, it is understood that, where the relative position of the groups is not specified, any positional isomer is intended to be within the scope of the embodiment. For example, “methoxyphenyl” includes phenyl having a methoxy substituent that may be ortho, meta, or para to the ring containing X1. “Difluorophenyl” includes phenyl having two fluoro substituents that may be ortho, meta, or para to each other, and either of which may be ortho, meta, or para to the ring containing X1. Where the relative position of the groups is specified, the substituent is merely exemplary of any positional isomer having such groups, and such positional isomers are intended to be within the scope of the embodiment.

In another exemplary embodiment of the invention,

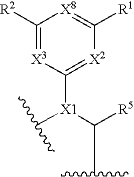

has the structure

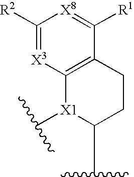

As an example of this embodiment, the aromatic ring containing X³ and X⁸ may be substituted with one or more groups each independently selected from bromo, chloro and methoxy.

Exemplary embodiments of the invention also include embodiments wherein R¹⁷ is selected from the group consisting of the following substituents:

cycloalkyl such as cyclopropyl;

alkyl, such as methyl or ethyl;

alkyl substituted with halogen, such as fluoroethyl or fluoromethyl; and alkyl substituted with alkoxy, such as methoxyethyl or methoxymethyl.

Exemplary embodiments of the invention also include embodiments wherein each of R¹¹, R¹², R¹³ and R¹⁴ is independently selected from the group consisting of the following substituents:

fluoro, bromo, cyano, chloro, alkoxy such as methoxy, aryl such as phenyl, amino, alkylamino, dialkylamino, carboxy, carboxyalkyl, carbonylamino, alkylcarbonyl, wherein the alkyl is optionally substituted with one or more alkoxy which is optionally substituted with aryl; cycloalkylcarbonyl; heteroaryl optionally substituted with one or more alkyl or one or more alkoxy, such as methoxypyridinyl; CO-heteroaryl optionally substituted with one or more alkyl or one or more alkoxy; aryl optionally substituted with one or more alkyl or one or more alkoxy or one or more halogen; alkyl such as methyl, and alkyl substituted with aryl, hydroxyl, alkoxy, cycloalkyl or halogen.

In one embodiment of the invention, R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring

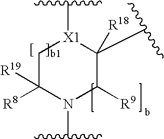

are cis-fused.

In one embodiment of the invention, R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring

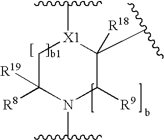

are trans-fused.

In another embodiment of the invention, the compound of formula I is an optically active compound of the formula

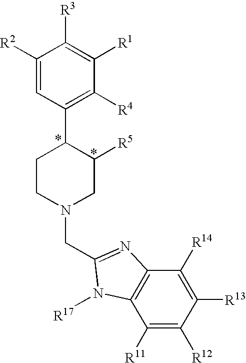

wherein R¹⁷, R¹¹, R¹², R¹³ and R¹⁴ are as defined in formula I; R¹ and R² are each independently halogen or hydrogen; R³ is halogen, hydrogen, alkyl optionally substituted with halogen, or alkoxy optionally substituted with halogen; R⁴ is halogen, hydrogen, alkyl optionally substituted with halogen, or alkoxy; and R⁵ is alkyl optionally substituted with aryloxy, wherein each of the carbons marked with an asterisk independently has the (R) configuration or the (S) configuration, provided that the R⁵ group and the phenyl group substituted with R¹, R², R³ and R⁴ are cis to each other.

In another embodiment of the invention, the compound of formula I is an optically active compound of the formula

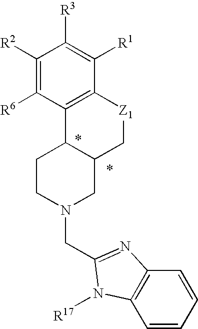

wherein R¹⁷ is as defined in formula I; Z₁ is O or CH₂; R¹ and R² are each independently halogen, hydrogen, or OR¹⁰¹ wherein R¹⁰¹ is alkyl or cycloalkyl, R³ is halogen, hydrogen, alkyl optionally substituted with halogen, or alkoxy optionally substituted with halogen; R⁶ is halogen, hydrogen, alkyl optionally substituted with halogen, or alkoxy wherein each of the carbons marked with an asterisk independently has the (R) configuration or the (S) configuration.

Exemplary compounds according to the invention include the compounds disclosed in Table 7 herein.

The compounds of formula I are useful for the treatment or prevention of a variety of neurological and psychiatric disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington'Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder, and obsessive compulsive disorder, mood disorders (including depression, mania, bipolar disorders), trigeminal neuralgia, hearing loss tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder. Accordingly, in one embodiment, the invention provides a method for treating or preventing a condition in a mammal, such as a human, selected from the conditions above, comprising administering a compound of formula I to the mammal. The mammal is preferably a mammal in need of such treatment or prevention. As an example, the invention provides a method for treating or preventing a condition selected from migraine, anxiety disorders, schizophrenia, and epilepsy. Exemplary anxiety disorders are generalized anxiety disorder, social anxiety disorder, panic disorder, post-traumatic stress disorder and obsessive-compulsive disorder.

In another embodiment the present invention provides methods of treating or preventing neurological and psychiatric disorders associated with glutamate dysfunction, comprising: administering to a patient in need thereof an amount of a compound of formula I effective in treating or preventing such disorders. The compound of formula I is optionally used in combination with another active agent. Such an active agent may be, for example, a metabotropic glutamate receptor agonist.

The invention is also directed to a pharmaceutical composition comprising a compound of formula I, and a pharmaceutically acceptable carrier. The composition may be, for example, a composition for treating or preventing a condition selected from the group consisting of acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder, wherein the composition contains an amount of the compound of formula I that is effective in the treatment or prevention of such conditions. The composition may be, as another example, a composition comprising an mGluR-2 antagonizing amount of the compound of formula I.

The composition may also further comprise another active agent. Such an active agent may be, for example, a metabotropic glutamate receptor agonist.

### DETAILED DESCRIPTION OF THE INVENTION

This detailed description of embodiments is intended only to acquaint others skilled in the art with Applicants' invention, its principles, and its practical application so that others skilled in the art may adapt and apply the invention in its numerous forms, as it may be best suited to the requirements of a particular use. This invention, therefore, is not limited to the embodiments described in this specification, and may be variously modified.

### Abbreviations and Definitions

TABLE A
Abbreviations

| | |
|---|---|
| 1-HOAT | 1-hydroxy-7-azabenzotriazole |
| 1-HOBt | 1-hydroxybenzotriazole hydrate |
| ADP | Adenosine diphosphate (the natural ligand of P2Y12) |
| AMP | Adenosine monophospate |
| ASA | Acetylsalicylic acid |
| ATP | Adenosine triphosphate |
| Bn | Benzyl group |
| Boc | tert-butoxycarbonyl |
| BOP-Cl | bis(2-oxo-3-oxazolidinyl)phosphinic chloride |
| br | Broad |
| BSA | Bovine serum albumin |
| Cbz | benzyloxycarbonyl |
| CD₃OD | Deuterated methanol |
| CDCl₃ | Deuterated chloroform |
| CDI | 1,1′-carbonyldiimidazole |
| d | Doublet |
| DBN | 1,5-diazabicyclo[4.3.0]non-5-ene |
| DBU | 1,8-diazabicyclo[5.4.0]undec-7-ene |
| DCC | 1,3-dicyclohexylcarbodiimide |
| DCM | dichloromethane |
| DMC | 2-chloro-1,3-dimethylimidazolinium chloride |
| dd | Doublet of doublets |
| DEPC | diethyl cyanophosphonate |
| DIEA | diisopropylethylamine |
| DMF | N,N-dimethylformamide |
| DMSO | dimethyl sulphoxide |
| DPBS | Dulbecco's Phosphate Buffered Saline |
| EBSS | Earle's Balanced Salt Solution |
| EDC | 1-(3-dimethylaminopropyl)-3-ethylcarbodiimide |
| | hydrochloride |
| EDTA | ethylenediaminetetraacetic acid |
| EGTA | ethyleneglycol-bis(β-aminoethyl)-N,N,N′,N′-tetraacetic |
| | Acid |
| ESI | Electrospray Ionization for mass spectrometry |
| Et₃N | triethylamine |
| EtOAc | ethyl acetate |
| EtOH | ethanol |
| FBS | Fetal bovine serum |
| Fmoc | Fluorene methyloxycarbonyl |
| HATU | O-(7-azabenzotriazol-1-yl)-N,N,N′,N′-tetramethyluronium |
| | hexafluorophosphate |
| HBTU | O-benzotriazol-1-yl-N,N,N′,N′-tetramethyluronium |
| | hexafluorophosphate |
| HCl | Hydrochloric acid |
| HEK | Human embryonic kidney |
| HEPES | 4-(2-hydroxyethyl)-1-Piperazineethane sulfonic acid |
| HOBT | 1-hydroxybenzotriazole |
| HRMS | High Resolution Mass Spectroscopy (electrospray |
| | ionization positive scan) |
| K₃PO₄ | Potassium phosphate |
| LCMS | Liquid Chromatography - Mass Spectroscopy |
| LRMS | Low Resolution Mass Spectroscopy (electrospray or |
| | thermospray ionization positive scan) |
| LRMS (ES) | Low Resolution Mass Spectroscopy (electrospray ionization |
| | negative scan) |
| m | Multiplet |
| m/z | Mass spectrum peak |
| MEM | Minimum essential medium |
| MeOH | methanol |
| MHz | Megahertz |
| MS | Mass spectroscopy |
| NaH | Sodium hydride |
| NMM | N-methylmorpholine |
| NMP | 1-methyl-2-pyrrolidinone |
| NMR | Nuclear Magnetic Resonance |
| PG | Protecting group. Exemplary protecting groups include Boc, |
| | Cbz, Fmoc and benzyl |
| Pg. | Page |
| PPP | Platelet poor plasma |
| PRP | Platelet rich plasma |
| q | Quartet |
| Rpm | Revolutions per minute |
| s | Singlet |
| t | Triplet |
| TFA | trifluoroacetic acid |
| THF | Tetrahydrofuran |
| TLC | Thin layer chromatography |
| Vol. | Volume |
| δ | Chemical shift |
| DEA | Diethylamine |

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl, pentyl, iso-amyl, hexyl and the like.

The term “alkenyl” refers to a linear or branched-chain hydrocarbyl substituent containing one or more double bonds and from two to twenty carbon atoms; in another embodiment, from one to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” embraces substituents having “cis” and “trans” orientations, or alternatively, “E” and “Z” orientations.

The term “benzyl” refers to methyl radical substituted with phenyl, i.e., the following structure:

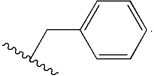

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”, indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur.

The term “cycloalkyl” refers to a saturated carbocyclic substituent having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substitutents, unless otherwise specified, are each bound to a carbon atom of the cycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclopentyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the aryl group. When such a fused aryl group is substituted with one more substituents, the one or more substitutents unless otherwise specified, are each bound to an aromatic carbon of the fused aryl group. The fused C₄-C₁₀ carbocyclic or 4-10-membered heterocyclic ring may be optionally substituted with halogen, C₁-C₆ alkyl, C₃-C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (e.g., alkyl, alkenyl, cycloalkyl, cycloalkenyl, aryl, etc.) is indicated by the prefix “Cₓ-C_{y},” wherein x is the minimum and y is the maximum number of carbon atoms in the substitutent. Thus, for example, “C₁-C₆-alkyl” refers to an alkyl substituent containing from 1 to 6 carbon atoms. Illustrating further, C₃-C₆-cycloalkyl refers to saturated cycloalkyl containing from 3 to 6 carbon ring atoms.

In some instances, the number of atoms in a cyclic substituent containing one or more heteroatoms (e.g., heteroaryl or heterocycloalkyl) is indicated by the prefix “X-Y-membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one or more heteroatoms, in the cyclic moiety of the heterocycloalkyl.

The term “hydrogen” refers to hydrogen substituent, and may be depicted as —H.

The term “hydroxy” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO₂.

The term “cyano” (also referred to as “nitrile”) —CN, which also may be depicted

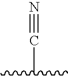

The term “carbonyl” means —C(O)—, which also may be depicted as:

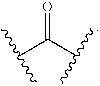

The term “amino” refers to —NH₂—.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

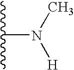

and dialkylamino such as dimethylamino, (exemplified by the formula

—N(CH₃)₂, which may also be depicted:

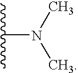

The term “aminocarbonyl” means —C(O)—NH₂, which also may be depicted as:

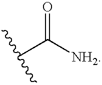

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfluoroalkyl” refers to an alkyl substituent wherein a fluorine substituent is in the place of each hydrogen substituent. Examples of perfluoroalkyl substituents include trifluoromethyl (—CF₃), perfluorobutyl, perfluoroisopropyl, perfluorododecyl, and perfluorodecyl. To illustrate further, the term “perfluoroalkoxy” refers to an alkoxy substituent wherein each hydrogen substituent is replaced with a fluorine substituent. Examples of perfluoroalkoxy substituents include trifluoromethoxy (—O—CF₃), perfluorobutoxy, perfluoroisopropoxy, perfluorododecoxy, and perfluorodecoxy.

The term “oxo” refers to ═O.

The term “oxy” refers to an ether substituent, and may be depicted as —O—.

The term “alkoxy” refers to an alkyl linked to an oxygen, which may also be represented as

—O—R, wherein the R represents the alkyl group. Examples of alkoxy include methoxy, ethoxy, propoxy and butoxy.

The term “alkythio” means —S-alkyl. For example, “methylthio” is —S—CH₃. Other examples of alkylthio include ethylthio, propylthio, butylthio, and hexylthio.

The term “alkylcarbonyl” means —C(O)-alkyl. For example, “ethylcarbonyl” may be depicted as

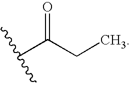

Examples of other alkylcarbonyl include methylcarbonyl, propylcarbonyl, butylcarbonyl, pentylcarbonyl, and hexylcarbonyl.

The term “aminoalkylcarbonyl” means —C(O)-alkyl-NH₂. For example, “aminomethylcarbonyl” may be depicted as:

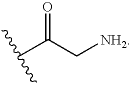

The term “alkoxycarbonyl” means —C(O)—O-alkyl. For example, “ethoxycarbonyl” may be depicted as

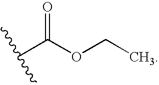

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The terms “thio” and “thia” mean a divalent sulfur atom and such a substituent may be depicted as —S—. For example, a thioether is represented as “alkyl-thio-alkyl” or, alternatively, alkyl-S-alkyl.

The term “thiol” refers to a sulfhydryl substituent, and may be depicted as —SH.

The term “thione” refers to ═S.

The term “sulfonyl” refers to —S(O)₂—, which also may be depicted as:

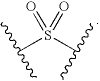

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “aminosulfonyl” means —S(O)₂—NH₂, which also may be depicted as

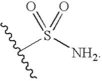

The term “sulfinyl” or “sulfoxido” means —S(O)—, which also may be depicted as:

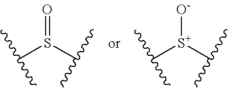

Thus, for example “alkylsulfinylalkyl” or “alkylsulfoxidoalkyl” refers to alkyl-S(O)-alkyl. Exemplary alkylsulfinyl groups include methylsulfinyl, ethylsulfinyl, butylsulfinyl, and hexylsulfinyl.

The term “heterocycloalkyl” refers to a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g. nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituent with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆-C₁₀ aromatic ring or to a 5-10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one more substituents, the one or more substitutents, unless otherwise specified, are each bound to a heteroatom of the heterocycloalkyl group or to a carbon atom of the heterocycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “heteroaryl” refers to an aromatic ring structure containing from 5 to 14 ring atoms in which at least one of the ring atoms is a heteroatom (i.e., oxygen, nitrogen, or sulfur), with the retaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls include furanyl, dihydrofuranyl, tetrahydrofuranyl, thiophenyl (also known as “thiofuranyl”), dihydrothiophenyl, tetrahydrothiophenyl, pyrrolyl, isopyrroyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiadiazolyl, oxathiazolyl, oxadiazolyl (including oxadiazolyl, 1,2,4-oxadiazolyl (also known as “azoximyl”) 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl, dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)); oxazinyl (including 1,2,3-oxazinyl, 1,3,2-oxazinyl, 1,3,6-oxazinyl (also known as “pentoxazolyl”), 1,2,6-oxazinyl or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryl include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”, anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl 1,4,2-benzoxazinyl 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substitutents, unless otherwise specified, are each bound to an aromatic carbon of the heteroaryl group or to a heteroatom of the heteroaryl group. The fused C₄-C₁₀ carbocyclic or 4-10-membered heterocyclic ring may be optionally substituted with halogen, C₁-C₆ alkyl, C₃-C₁₀ cycloalkyl, or ═O.

The term “ethylene” refers to the group —CH₂—CH₂—.

The term “ethynelene” refers to the group —CH═CH—.

The term “propylene” refers to the group —CH₂—CH₂—CH₂—CH₂—.

The term “butylene” refers to the group —CH₂—CH₂—CH₂—CH₂—.

The term “methylenoxy” refers to the group —CH₂—O—.

The term “methylenethioxy” refers to the group —CH₂—S—.

The term “methylenamino” refers to the group —CH₂—N(H)—

The term “ethylenoxy” refers to the group —CH₂—CH₂—O—.

The term “ethylenethioxy” refers to the group —CH₂—CH₂—S—

The term “ethylenamino” refers to the group —CH₂—CH₂—N(H)—.

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituents is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen

A prefix attached to a multi-moiety substituent only applies to the first moiety. To illustrate, the term “alkylcycloalkyl” contains two moieties: alkyl and cycloalkyl. Thus, a C₁-C₆-prefix on C₁-C₆-alkylcycloalkyl means that the alkyl moiety of the alkylcycloalkyl contains from 1 to 6 carbon atoms; the C₁-C₆-prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution may only occur on the alkyl moiety, the substituent would be described as “alkoxyhaloalkyl.” If the halogen substitution may occur on both the alkyl moiety and the alkoxy moiety, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be depicted as

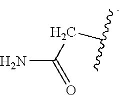

In a substituent trifluoromethylaminocarbonyl, the carbonyl moiety is attached to the remainder of the molecule, where the substituent may also be depicted as

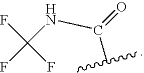

If substituents are described as being “independently selected” from a group, each substituent is selected independent of the other. Each substituent therefore may be identical to or different from the other substituent(s).

### Isomers

When an asymmetric center is present in a compound of formulae I through IV, hereinafter referred to as the compound of the invention, the compound may exist in the form of optical isomers (enantiomers). In one embodiment, the present invention comprises enantiomers and mixtures, including racemic mixtures of the compounds of formulae I through IV. In another embodiment, for compounds of formulae I through IV that contain more than one asymmetric center, the present invention comprises diastereomeric forms (individual diastereomers and mixtures thereof) of compounds. When a compound of formulae I through IV contains an alkenyl group or moiety, geometric isomers may arise.

### Tautomeric Forms

The present invention comprises the tautomeric forms of compounds of formulae I through IV. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context) the salt preferably is pharmaceutically acceptable. The term “pharmaceutically acceptable salt” refers to a salt prepared by combining a compound of formulae I-V with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g. methyl, ethyl, propyl, and butyl chlorides, bromides, and iodides), dialkyl sulfates (e.g., dimethyl, diethyl, dibuytl, and diamyl sulfates), long chain halides (,e.g., decyl, lauryl, myristyl, and stearyl chlorides, bromides, and iodides), arylalkyl halides (e.g., benzyl and phenethyl bromides), and others.

In one embodiment, hemisalts of acids and bases may also be formed, for example, hemisulphate and hemicalcium salts.

The compounds of the invention may exist in both unsolvated and solvated forms. A “solvate” as used herein is a nonaqueous solution or dispersoid in which there is a noncovalent or easily dispersible combination between solvent and solute, or dispersion means and disperse phase

### Prodrugs

Also within the scope of the present invention are so-called “prodrugs” of the compound of the invention. Thus, certain derivatives of the compound of the invention which may have little or no pharmacological activity themselves can, when administered into or onto the body, be converted into the compound of the invention having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association), Prodrugs in accordance with the invention can, for example, be produced by replacing appropriate functionalities present in the compounds of any of formulae I through IV with certain moieties known to the skilled in the art as “pro-moieties” as described, for example, in “Design of Prodrugs” by H Bundgaard (Elsevier, 1985).

### Isotopes

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁶S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium. i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

### Administration and Dosing

Typically, a compound of the invention is administered in an amount effective to treat or prevent a condition as described herein. The compounds of the invention are administered by any suitable route in the form of a pharmaceutical composition adapted to such a route, and in a dose effective for the treatment or prevention intended. Therapeutically effective doses of the compounds required to treat or prevent the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-tree injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration, and the activity of the particular compound employed. Thus the dosage regimen may vary widely. Dosage levels of the order from about 0.01 mg to about 100 mg per kilogram of body weight per day are useful in the treatment or prevention of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 10 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from About 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment or prevention of the conditions recited herein.

### Pharmaceutical Compositions

For the treatment or prevention of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment or prevention intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of Formulae I through IV are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975, Liberman, et al., Eds., Pharmaceutical Dosage Forms, Marcel Decker, New York, N.Y., 1980, and Kibbe, et al. Eds. Handbook of Pharmaceutical Excipients (3^{rd} Ed.), American Pharmaceutical Association, Washington, 1999.

### Co-Administration

The compounds of the present invention can be used, alone or in combination with other therapeutic agents, in the treatment or prevention of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment or prevention described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Intermediates

In another embodiment, the invention relates to the novel intermediates useful for preparing the compounds of the invention

### General Synthetic Schemes

The compounds of the formula I may be prepared by the methods described below, together with synthetic methods known in the art of organic chemistry, or modifications and derivatisations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981, T. W. Greene and P. G. M. Wuts, Protective Groups in Organic Chemistry, John Wiley & Sons, 1991, and T. W. Greene and P. G. M. Wuts, Protective Groups in Organic Chemistry. John Wiley & Sons, 1999, which are hereby incorporated by reference.

Compounds of formula I, or their pharmaceutically acceptable salts, can be prepared according to the reaction Schemes discussed hereinbelow. Unless otherwise indicated, the substituents in the Schemes are defined as above. Isolation and purification of the products is accomplished by standard procedures, which are known to a chemist of ordinary skill.

The following schemes are exemplary of the processes for making compounds of formula I. In the schemes below, the numerals used, including numerals from (I) to (V), are used for convenience to designate the formulae in the schemes. The use of numerals from (I) to (V) in the schemes below is not intended to imply that the compounds designated by such numerals correspond to the compounds of formulae I-V that are disclosed hereinabove and that are recited in the appended claims.

Scheme I illustrates a method for the preparation of compounds of formula I, where R¹ to R¹⁰ and X¹ to X⁸ are defined as above.

Referring to scheme I, a compound of formula (I) can be synthesized by treating secondary amine of formula (II) with the aldehydes of formula (III) in the presence of suitable reducing agents such as NaBH(OAc)₃, or Na(CN)BH₃ in solvents such as methylene chloride, dichloroethane, DMF or THF, at about room temperature. Other suitable conditions for this transformation include treatment of the amine of formula (II) with aldehydes of formula (III) in solvents such as methanol or ethanol at room temperature, followed by treatment with reducing agents such as NaBH₄ or NaCNBH₃, which also produce the desired compounds of formula (I). Alternatively, a compound of formula (I) can be synthesized by alkylating the amine of formula (II) with reagents of formula (IV), wherein X is a good leaving group such as Cl, Br, I, mesylate or tosylate, in the presence of a suitable base, such as, but not limited to, diethylpropylamine, sodium carbonate, potassium carbonate, or sodium ethoxide, in solvents such THF, DMF or DMSO, at elevated temperature around 40° C. to 180° C. with or without microwave heating.

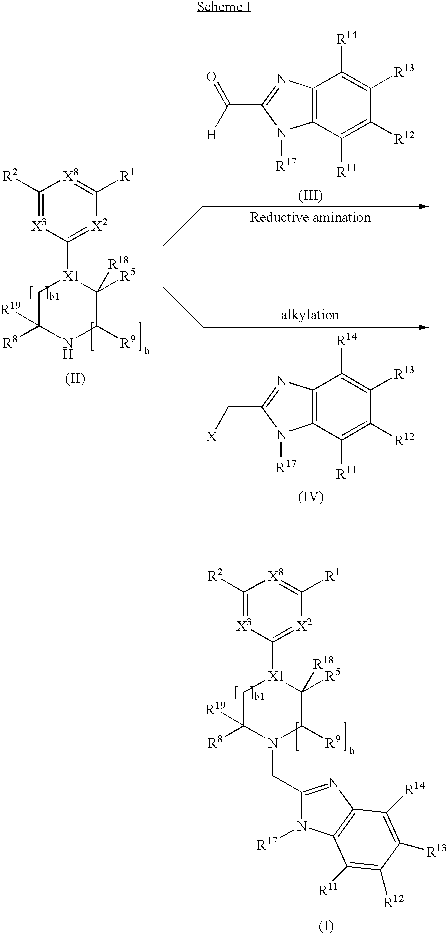

Aldehydes of formula (III) are either commercially available or can be prepared, but not limited to, by a general procedure illustrated by scheme II, wherein R¹⁷, and R¹¹ through R¹⁴ are defined as above. Referring to scheme II below, substituted 2-halo-nitrobenzene (V) can be treated with primary amine of formula (VI) in the presence of a suitable base such as potassium carbonate and the like and in a suitable solvent such as dichloromethane at a reaction temperature ranging from room temperature to 100° C. to give aniline of formula (VII). Reduction of the nitro group using well-precedented conditions, such as Pd/C under hydrogen or Fe/EtOH/CaCl₂, can yield dianiline of formula (VII). The imidazole ring can then be formed by treating dianiline (VII) with acetimidates of formula (XII) in the presence of acetic acid, in a suitable solvent such as MeOH. The acetal of compound (XI) can be deprotected with acids such as HCl to yield the desired aldehyde of formula (III). Alternatively, dianiline (VIII) can be condensed with glycolic acid under strong acidic conditions, such as aqueous hydrochloric acid, at elevated temperature such as reflux. The resultant alcohol of formula (X) can then be oxidized using a suitable oxidation reagent, such as MnO₂ in a suitable solvent such as methylene chloride, to yield the desired aldehyde of formula (III). In addition, dianiline (VII) can cyclize with triethylorthoacetate in a suitable solvent such as ethanol at elevated temperature with or without microwave heating to produce imidazole of formula (IX), which can be subsequently oxidized to the desired aldehyde of formula (II) using selenium dioxide. Other known literature procedures on synthesis of methylbenzimidazole aldehydes or small variations of the synthesis described above can also be used.

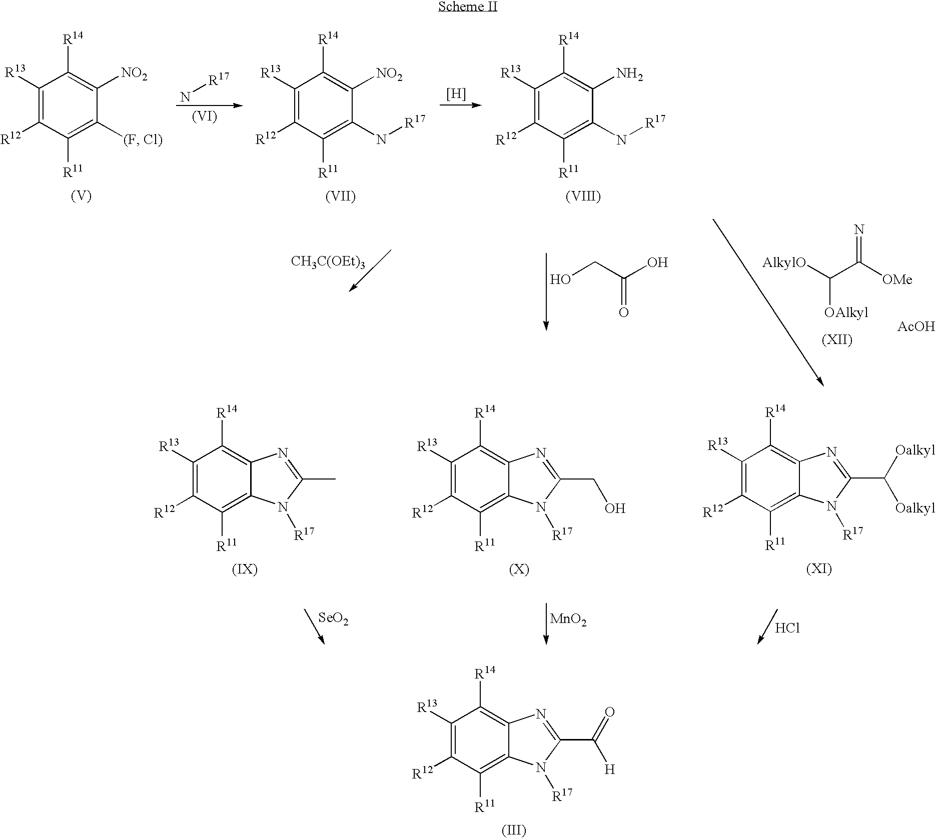

Scheme III Illustrates the synthesis of imidazole of formula (IV), wherein R¹¹ to R¹⁷ are defined as above and X is Cl, Br, I, OMs or OTs. Referring to scheme III, condensation of dianiline (VIII) with chloroacetic acid under strong acidic conditions, such as 6N HCl, at elevated temperature, give the imidazole of formula (IV), wherein X is Cl. Alternatively, dianiline (VIII) can be condensed with glycolic acid to yield alcohol (X). The alcohol then can be converted to compound of formula (IV), wherein X is Br, I, mesylate or tosylated using well established literature procedures (Comprehensive Organic Transformations: a Guide to Functional Group Preparations, Larock, R. C., VCH Publishers Inc, 1989, ISBN 0-89573-710-8).

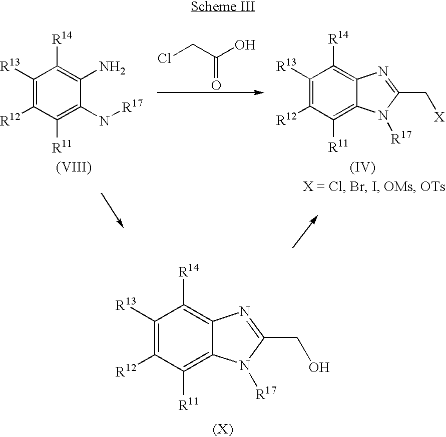

Scheme IV illustrates the synthesis of compound of formula (XIX), wherein R³ to R¹⁷ are defined as above and R is hydrogen or any one of the substituents R¹-R⁴ and R⁶ as defined in formula I. Boc-protected piperidinone (XIII), either commercially available or readily prepared from commercial precursors, isss treated with a suitable base, such as diethylisopropylamine, triethylamine and the like, in the presence of a triflic source such as triflic anhydride to form enol triflate of formula (XIV). Suzuki coupling of enol triflate (XIV) and boronic acid (XV), with a catalyst such as palladium (0) tetrakis(triphenylphosphine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenylphosphino)ferrocene]palladium (II) dichloromethane adduct, in the presence or absence of a base such as potassium phosphate, potassium acetate, sodium acetate, cesium acetate, sodium carbonate, lithium carbonate, potassium carbonate, cesium fluoride or cesium carbonate, preferably sodium carbonate, give olefin (XVI). This reaction is typically carried out in an inert solvent such as dimethyl ethylene glycol ether (DME), 1,4-dioxane, acetonitrile, methyl sulfoxide, tetrahydrofuran, ethanol, methanol, 2-propanol, or toluene, in the presence or absence of from about 1%-about 10% water, preferably about 5% water, with or without microwave assisted heating at a temperature from about 0° C. to about 200° C., preferably from about 60° C. to about 100° C. Hydrogenation of resultant olefin (XVI) under hydrogen in the presence of a suitable catalyst, such as Pd/C, Pd(OH)₂ or PtO₂, yield aryl piperidine (XVII). Removal of Boc protecting group under acidic conditions, such as trifluoroacetic acid or HCl, give amines of formula (XVIII). Amine (XVIII) can then undergo reductive amination with aldehyde (III) or alkylation with reagents of formula (IV), as described in Scheme I, to give the compounds of formula (XIX)

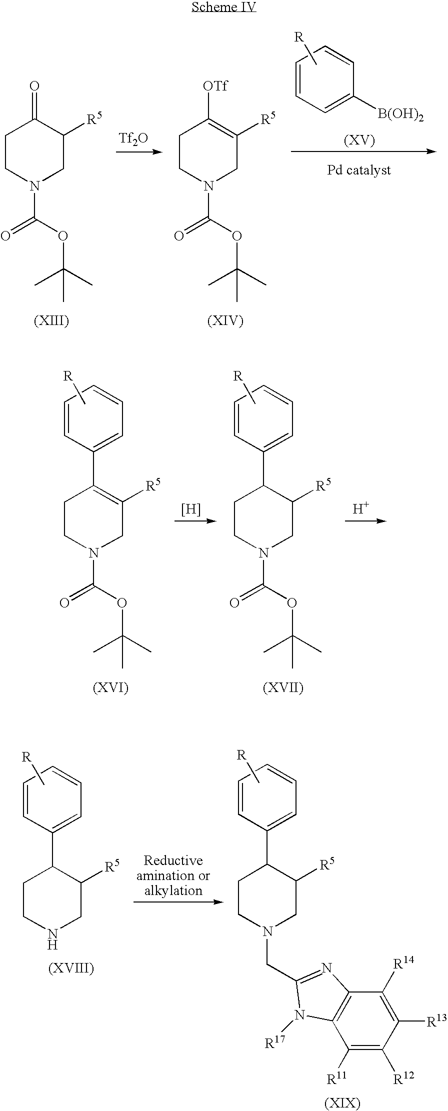

Alternatively, arylpiperidine of formula (XVII) can be synthesized as illustrated in Scheme V. Referring to Scheme V, piperidinone (XIII) can be treated with an aryllithium or aryl Grinard species of formula (XX) to yield alcohol of formula (XXI). Dehydration of alcohol (XXI) under strong acidic conditions, such as trifluoroacetic acid or aqueous HCl solution, yields a mixture of olefin isomers (XXIIa) and (XXIIb). Subsequent hydrogenation of the olefin (XXIIa, b) using a suitable catalyst, such as Pd/C, PtO₂ or Pd(OH)₂, under hydrogen in a suitable solvent, such as ethanol, methanol or ethylacetate, followed by deprotection yield arylpiperidine of formula (XVIII), which then can be further derivatized to give compound of formula (XIX) as described in Scheme IV. Alternatively, treating alcohol (XXI) with ethyl chloroformate yields carbonate (XVIII), which upon heating in a suitable high boiling point and inert solvent, such as decalin, give olefin of formula (XXIIb). Subsequent hydrogenation of the olefin and deprotection give aryl piperidine of formula (XVIII).

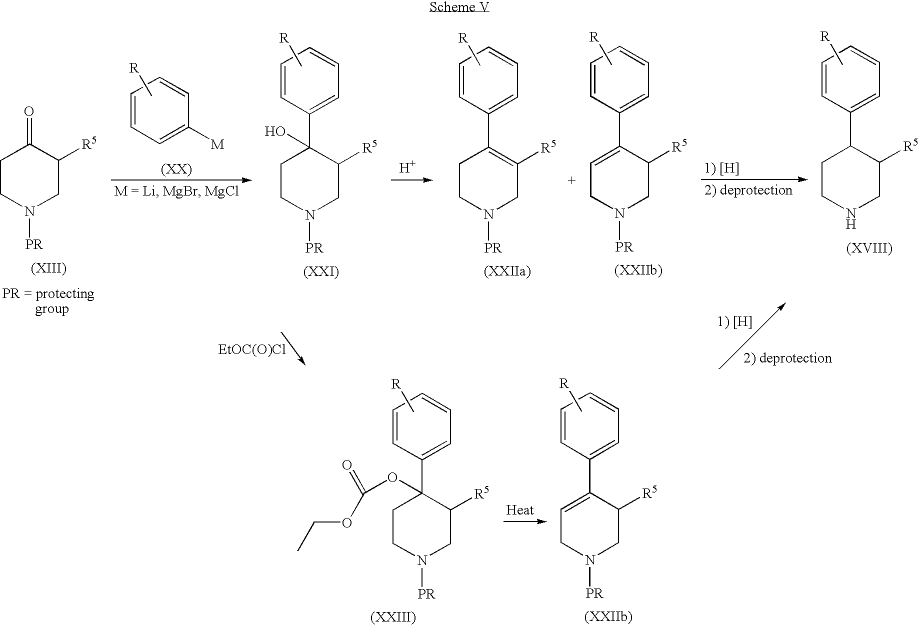

Scheme VI illustrates the synthesis of compounds of formula (XXVII), wherein R⁵, R⁸, R⁹, R¹¹-R¹⁴ and R¹⁷ are defined as above. R is hydrogen or any one of the substituents R¹-R⁴ and R⁶ as defined in formula I. Bromopyridine of formula (XXIV), either commercially available or easily prepared from commercial sources, can be coupled with boronic acid of formula (XV) to give aryl-pyridine (XXV). Suitable conditions for this Suzuki coupling reaction involve a catalyst, such as palladium (0) tetrakis(triphenylphosphine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenyl(phosphino(ferrocene]palladium (II) dichloromethane adduct, in the presence or absence of a base such as potassium phosphate, potassium acetate, sodium acetate, cesium acetate, sodium carbonate, lithium carbonate, potassium carbonate, cesium fluoride or cesium carbonate, preferably sodium carbonate. This reaction is typically carried out in an inert solvent such as dimethyl ethylene glycol ether (DME), 1,4-dioxane, acetonitrile, methyl sulfoxide, tetrahydrofuran, ethanol, methanol, 2-propanol, or toluene, in the presence or absence of from about 1% to about 10% water, preferably about 5% water, with or without microwave assisted heating at a temperature from about 0° C. to about 200° C., preferably from about 60° C. to about 100° C. Hydrogenation of the HCl salt of pyridine (XXV) under hydrogen in the presence of a suitable catalyst, such as Pd/C, Pd(OH)₂ or PtO₂, in a suitable solvent, such as ethanol, at elevated temperature, yield amine (XXVI). The resultant amine (XXVI) can then undergo reductive amination with aldehyde (III) or alkylation with reagents of formula (IV), as described in Scheme I, to give the compounds of formula (XXVII)

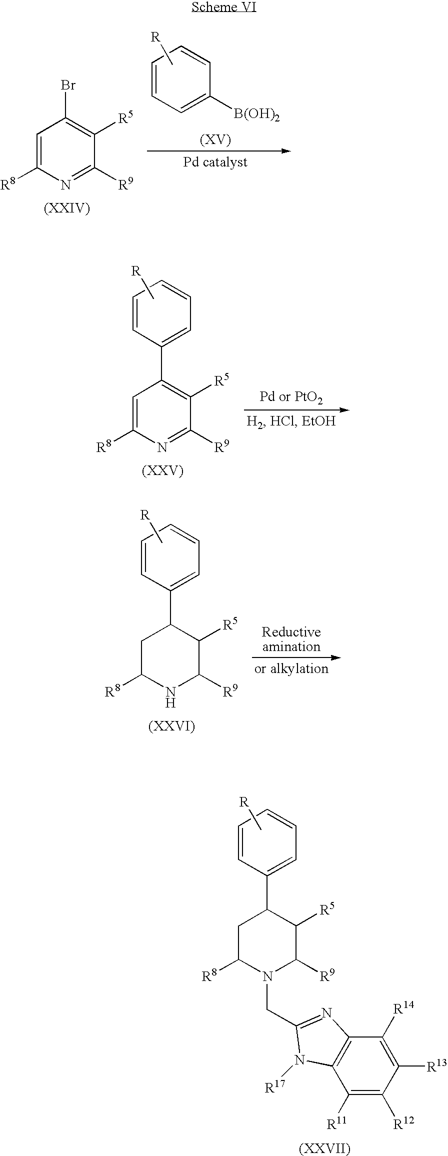

Scheme VII illustrates the synthesis of compounds of formula (XXXII) wherein R¹¹-R¹⁴, R¹⁷ and R¹⁰¹ are defined as above. R is hydrogen or any one of the substituents R¹-R⁴ and R⁶ as defined in formula I. Referring to Scheme VII, deprotection of methoxy group of arylpiperidine (XXVIII), prepared through methods described in Scheme IV-VI, yield phenol of formula (XXIX). Phenol (II) can be coupled with an alcohol of formula (XXX) in the presence of a suitable coupling reagent such as diethylazodicarboxylate (DEAD) and triarylphosphines, such as triphenylphosphine, in solvents such as THF or ether at or about room temperature, to produce the corresponding ether of formula (XXXI). The amine (XXXI) can then undergo reductive amination with aldehyde (III) or alkylation with reagents of formula (IV), as described in Scheme I, to give the compounds of formula (XXXII)

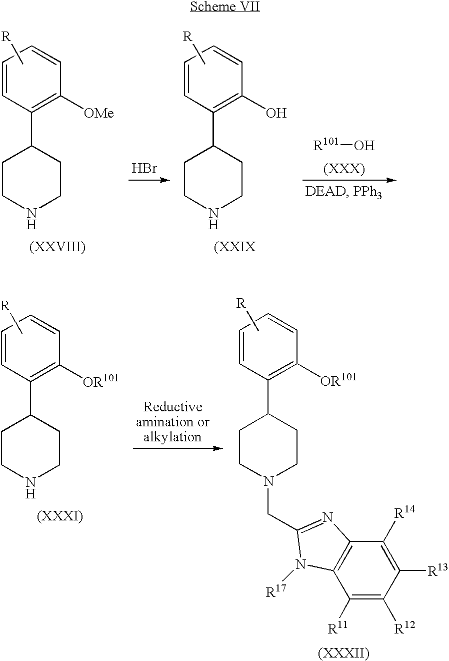

Scheme VIII illustrates the synthesis of compounds of formula (XXXV)-(XXXVII), wherein R⁵, R¹⁷, R⁴⁰¹ and R⁴⁰² are defined as above R is hydrogen or any one of the substituents R¹, R⁴ and R⁶ as defined in formula I. Referring to Scheme VIII, a compound of formula (XXXV) can be generated by treating a compound of formula (XXXIII), wherein R¹¹, R¹², R¹³ or R¹⁴ is chlorine, bromine, or iodine, with a reagent of formula (XXXIVa), wherein M is defined as a boronic acid, boronic ester, trialkylstanane, magnesium halogen, or zinc, with a palladium catalyst such as but not limited to palladium(0) tetrakis(triphenylphosphine), palladium(II) acetate, tris(dibenzylideneacetone) dipalladium(0), dichloro[1,1′-bis(diphenylphosphino)ferrocene]palladium (II) dichloromethane adduct, in the presence of a phosphine ligand such as but not limited to triphenylphosphine, tri-o-tolylphosphine, tri-tert-butylphosphine, 1,1′-bis(diphenyl-phosphino)ferrocene, 1,2-bis(diphenyl-phosphino)ethane, 1,3-bis(diphenylphosphino)-propane, 2,2′-bis(diphenylphosphino)-1,1′-binaphthyl (BINAP), in the presence or absence of a base such as but not limited to potassium or sodium acetate, sodium or potassium or cesium carbonate, potassium phosphate, cesium fluoride and sodium tert-butoxide. This reaction is typically carried out in an inert solvent such as 1,4-dioxane, ethyl ether, tetrahydrofuran (THF), benzene, toluene, DMF, DMSO in the presence or absence of 1%-10% water at a temperature from 0° C. to 200° C.

Compound of formula (XXXVI) is prepared by treating a compound of formula (XXXIII) with alcohol (XXXIVc) wherein R¹¹, R¹², R¹³ or R¹⁴ is chlorine, bromine, or iodine. The reaction is usually carried out in the presence of a copper salt such as, but not limited to, copper(I) chloride (CuCl) copper(II) triflate and copper(I) iodide (CuI), in the presence or absence of a ligand such as, but not limited to, 2,2,6,6-tetramethylheptane-3,5-dione (TMHD), 1,10-phenanthroline, 8-hydroxyquinoline, 2-aminopyridine and pentane-2,4-dione (acac), and in the presence or absence of a base such as cesium carbonate, potassium phosphate, potassium acetate, sodium acetate, cesium acetate, sodium carbonate, lithium carbonate, potassium carbonate, preferably cesium carbonate, using the reacting alcohol as solvent or in an inert solvent such as, but not limited to, benzene, toluene, xylene, N,N-dimethylformamide (DMF), dimethylsulfoxide (DMSO) and N-methylpyrrolidinone (NMP) at a temperature from about 0° C. to about 200° C.

Compound of formula (XXXVII) is prepared by treating a compound of formula (XXXIII), wherein R¹¹, R¹², R¹⁴ or R¹⁴ is chlorine, bromine, or iodine, with amine (XXXIVb). The reaction is carried out in the presence or absence of a palladium catalyst such as palladium(II) acetate, tris(dibenzylidene acetone)dipalladium(0), dichloro-[1,1′-bis(diphenylphosphino)ferrocene]palladium (II) dichloro methane adduct, in the presence or absence of a phosphine ligand such as BINAP, 1,3-bis(diphenylphosphino)-propane, or 1,1′-bis(diphenylphosphino)ferrocene, in the presence of a strong base such as sodium tert-butoxide in a suitable solvent such as toluene at a temperature from 60° C. to 110° C.

Compound of formula (XXXVa) is prepared by treating a compound of formula (XXXIII), wherein R¹¹, R¹², R¹⁴ or R¹⁴ is chlorine, bromine, or iodine, under carbon monoxide, typically 20-50 psi, in an alcoholic solvent, such as methanol or ethanol, in the presence of a suitable catalyst, such as but not limited to dichloro-[1,1′-bis-diphenylphosphino)ferrocene]palladium (II) dichloro methane adduct at a temperature from ambient to 80° C. The resultant ester functionality can be further derivatized to other functional groups.

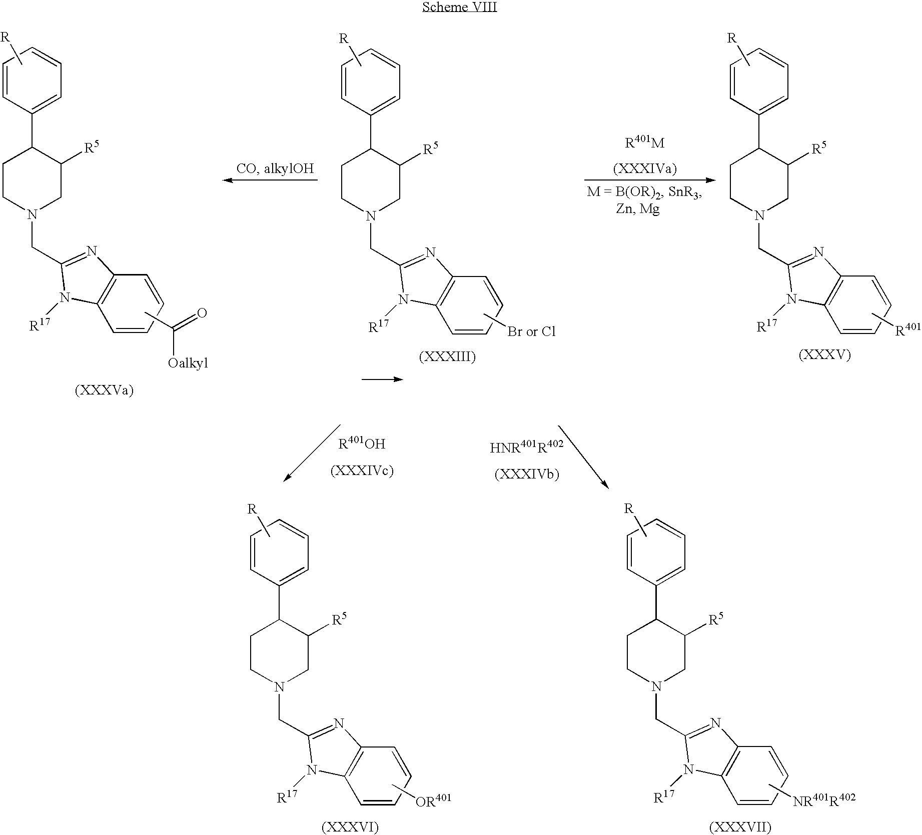

Scheme IX illustrates the synthesis of compounds of formulas (XLVII) and (XLIX), wherein R is hydrogen or any one of the substituents R¹-R⁴ and R⁶ as defined in formula I. Commercially available amino acid (XXXVIII) can be protected as a carbamate, here illustrated by benzyloxycarbonyl derivative (XXXIX) Carboxyl group can be converted to acid chloride, for example by treatment with oxalyl chloride in an inert solvent such as toluene optionally in the presence of catalytic amount of DMF. (XL) can be converted into aldehyde (XLI) directly by means of reducing conditions such as hydrogenation over palladium catalyst. Alternatively, acid chloride (XL) can be converted into alkyl ester (XLII) by reaction with an excess of the corresponding alcohol. The selective reduction of (XLII) to alcohol (XLIII) can be achieved, for example, by reaction with sodium borohydride in an alcoholic solvent. Convertion of primary alcohol (XLIII) to (XLI) can be accomplished by well known oxidation methods such as Swern oxidation and Dess-Martin oxidation. Spiroindoline derivatives (XLV) can be prepared by reacting hydrazines (XVIV) with protected aminoaldehydes such as (XVI) in an inert solvent such as toluene, dichloromethane or acetonitrile in the presence or absence of acidic catalysts exemplified by trifluoroacetic acid or zinc chloride followed by treatment with reducing agents such as sodium borohydride. The free amino group of (XLV) can be protected, for example as Boc (tert-butyloxycarbonyl) derivative illustrated by structure (XLVI). Cbz group can be removed using reducing conditions such as hydrogenation over palladium catalyst to afford monoprotected derivative (XLVII). Cbz group removal can also be preformed in a similar manner on the spiroindolines (XLV) to afford diamines (XLVII). The more reactive amino group of (XLVIII) can be selectively protected for example as Boc carbamate (XLIX).

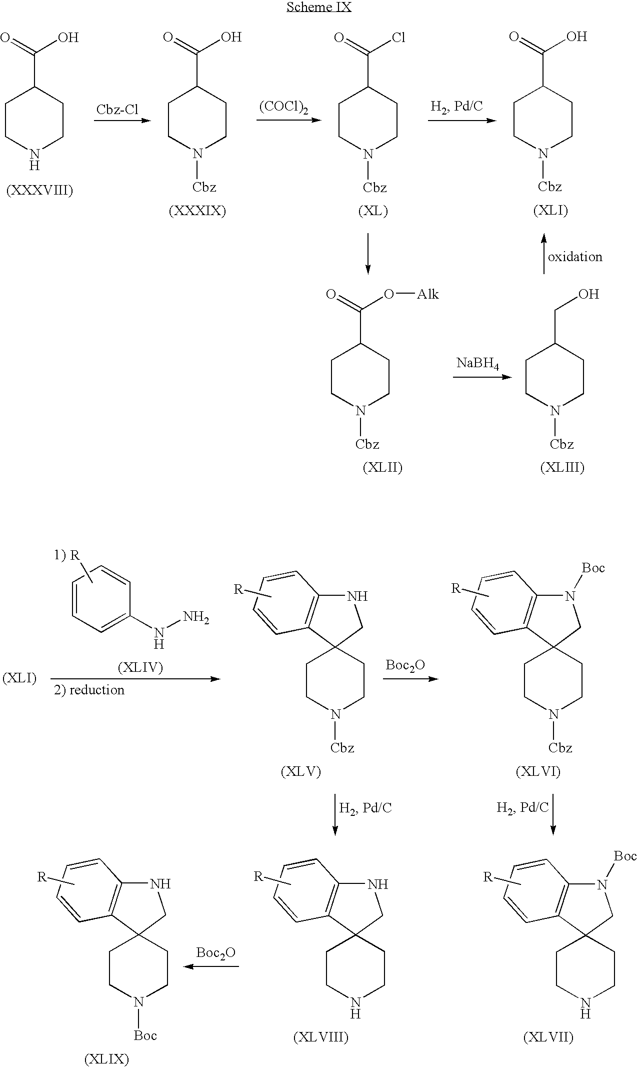

Scheme X illustrates an alternative synthesis of compounds of formula (XLIX), wherein R is hydrogen or any one of the substituents R³-R⁴ and R⁶ as defined in formula I. (2-Fluoroaryl)acetonitriles (LXVI) can be reacted with 2-chloro-N-(2-chloroethyl)-N-methylethanamine in the presence of a suitable base, such as, but not limited to, cesium carbonate, sodium hydride, potassium hexahydrodisilazide in solvents such as THF, DMF or DMSO to afford piperidines (LXVII). Spiroindoline compounds (LXVIII) can be obtained by reduction and spontaneous cyclization of (LXVII) using hydride reducing agents such as lithium aluminium hydride in solvents such as dimethoxyethane, dioxane or glyme optionally in the presence of alcohols such as methanol of ethanol. The free amino group can be protected as a carbamate derivative here illustrated by benzyloxycarbamate (LXIX) using conventional methods. Compounds of formula (LXX) can be obtained by selective demethylation by reaction with chloroethylchloroformate. The free amino group of (XLV) can be protected, for example as (tert-butyloxycarbonyl) derivative illustrated by structure (LXXI). Cbz group can be removed using reducing conditions such as hydrogenation over palladium catalyst to afford monoprotected derivative (XLIX).

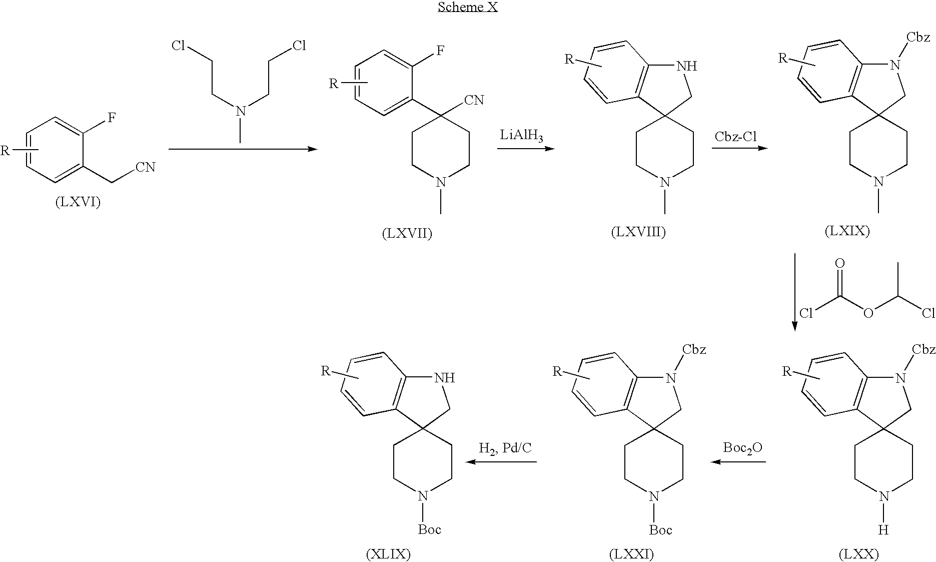

Scheme XI illustrates the synthesis of compounds of formulas (LII) and (LIII) where R′ is hydrogen or optionally substituted alkyl such as C₁-C₆ alkyl, R″ is optionally substituted aryl heteroaryl or alkyl such as C₁-C₆ alkyl and wherein R¹¹-R¹⁴ and R¹⁷ are defined as above. Spiroindoline derivative (XLVI) can undergo reductive amination with aldehyde (III) or alkylation with reagents of formula (IV), as described in Scheme I, to give the compounds of formula (L). Boc group can be removed by treatment with acidic reagents such as hydrochloric or trifluoroacetic acids in a solvent such as ether, dioxane or methanol. The compounds (LII) can be synthesized by treating secondary amine of formula (LI) with the corresponding aldehydes in the presence of suitable reducing agents such as NaBH(OAc)₃. Na(CN)BH₃, or formic acid in solvents such as methylene chloride, dichloroethane, DMF or THF, at about room temperature. Other suitable conditions for this transformation include treatment of the amine of formula (LI) with aldehydes in solvents such as methanol or ethanol at room temperature followed by treatment with reducing agents such as NaBH₄ or NaCNBH₃, which also produce the desired compounds of formula (LII). Alternatively, a compound of formula (LII) can be synthesized by alkylating the amine of formula (LI) with the corresponding alkylating agent in the presence of a suitable base, such as, but not limited to, diethylpropylamine, sodium carbonate, potassium carbonate, or sodium ethoxide, in solvents such THF, DMF or DMSO, at elevated temperature around 40° C. to 180° C. with or without microwave heating. Alternatively, amines (LI) can be converted to amides (LIII) by treatment with the corresponding carboxylic acids in the presence of activating agents such as, but not limited to, HBTU, HATU, carbonyldiimidazole, DMC, HOBT, and DCC in the presence or absence of a suitable base, such as, but not limited to, diethylpropylamine, sodium carbonate, potassium carbonate. Amides (LIII) can also be prepared by treatment of amines (LI) with the corresponding acid chlorides in the presence of a suitable base, such as, but not limited to, diethylpropylamine, sodium carbonate, potassium carbonate in solvents such as dichloromethane, THF, DMF or DMSO.

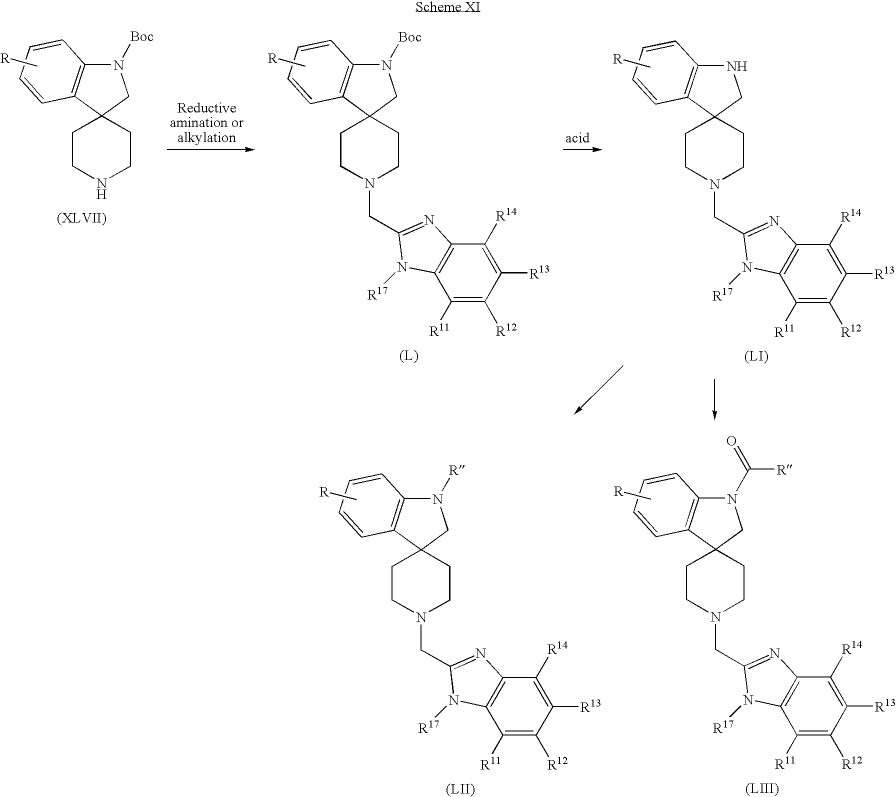

Scheme XII illustrates an alternative synthes of compounds of formulas (LII) and (LIII) where R′ is hydrogen or optionally substituted alkyl such as C₁-C₆ alkyl, R″ is optionally substituted aryl, heteroaryl or alkyl such as C₁-C₆ alkyl and wherein R¹¹-R¹⁴ and R¹⁷ are defined as above. The compounds (LIV) can be synthesized by treating secondary amine of formula (XLIX) with the corresponding aldehydes in the presence of suitable reducing agents such as NaBH(OAc)₃, Na(CN)BH₃, or formic acid in solvents such as methylene chloride, dichloroethane, DMF or THF, at about room temperature. Other suitable conditions for this transformation include treatment of the amine of formula (LI) with aldehydes in solvents such as methanol or ethanol at room temperature, followed by treatment with reducing agents such as NaBH₄ or NaCNBH₃, which also produce the desired compounds of formula (LII). Alternatively, a compound of formula (LIV) can be synthesized by alkylating the amine of formula (XLIX) with the corresponding alkylating agent in the presence of a suitable base, such as, but not limited to, diethylpropylamine, sodium carbonate, potassium carbonate, or sodium ethoxide, in solvents such THF, DMF or DMSO, at elevated temperature around 40° C. to 180° C. with or without microwave heating, Alternatively, amines (LXLIX) can be converted to amides (LVI) by treatment with the corresponding carboxylic acids in the presence of activating agents such as, but not limited to, HBTU, HATU, carbonyldiimidazole. DMC, HOBT, and DOC in the presence or absence of a suitable base, such as, but not limited to, diethylpropylamine, sodium carbonate, potassium carbonate. Amides (LVI) can also be prepared by treatment of amines (XLIX) with the corresponding acid chlorides in the presence of a suitable base, such as, but not limited to, diethylpropylamine, sodium carbonate, potassium carbonate in solvents such as dichloromethane, THF, DMF or DMSO. Free amine derivatives of formulas (LV) and (LVII) can be prepared by removal of the Boc group by treatment with acidic reagents such as hydrochloric or trifluoroacetic acids in a solvent such as ether or dioxane. Amines (LV), and (LVII) can undergo reductive amination with aldehyde (III) or alkylation with reagents of formula (IV), as described in Scheme I, to give the compounds of formulas (LII) and (LIII).

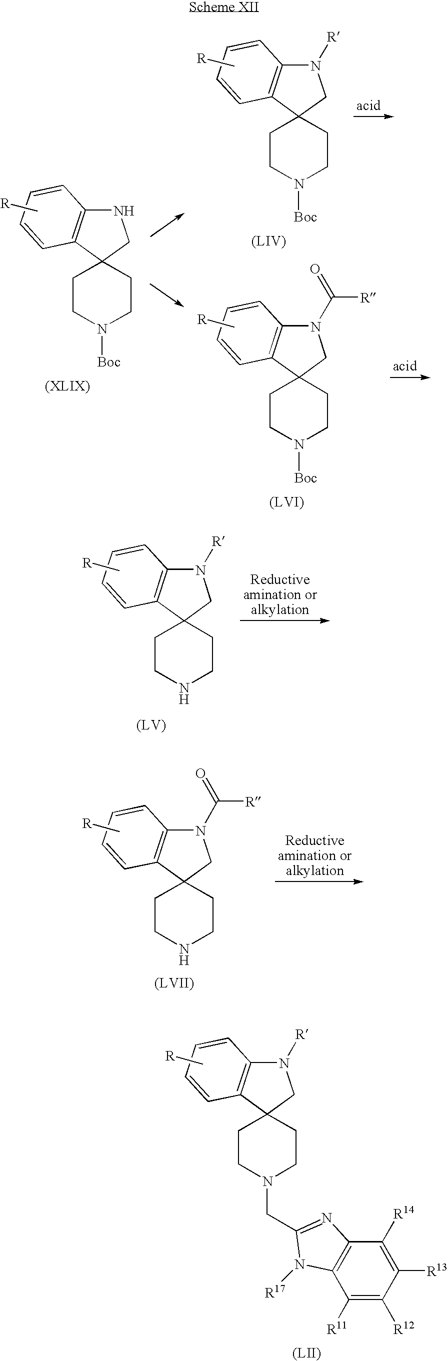

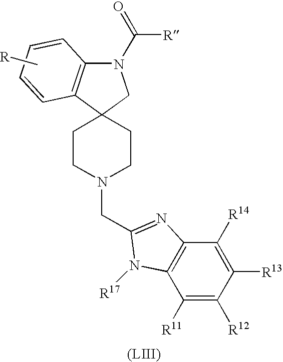

Scheme XIII illustrates a synthesis of F— or —OH substituted piperidines, wherein R is hydrogen or any one of the substituents R¹-R⁴ and R⁶ as defined in formula I. Referring to Scheme XII alcohol (LVIII) can be treated with a fluorinating reagent, such as diethylaminosulfurtrifluoride (DAST) or bis-(1-methoxyethyl)aminosulfurtrifluoride (BAST) in a suitable solvent such as methylene chloride, to give fluorinated compound of (LIX). Deprotection of Boc under acidic conditions yield 4-fluoro piperidine of formula (LX). For the synthesis of 3-fluoropiperidine (LXIII), olefin (LXI) can be converted to alcohol (LXII) via hydroboration reaction. A typical condition involves treating the substrate with borane dimethylsulfide complex, followed by hydrogen peroxide and sodium hydroxide aqueous solution. The resulting alcohol (LXII) can be deprotected under acidic condition to 3-hydroxyl piperidine (LXIV), or be fluorinated with DAST or BAST to give (LXIII), which upon deprotection to yield 3-fluoro piperidine (LXV). Reductive amination or alkylation of (LX), (LXIV) or LXV) according to Scheme I will yield desired compounds of formula (I).

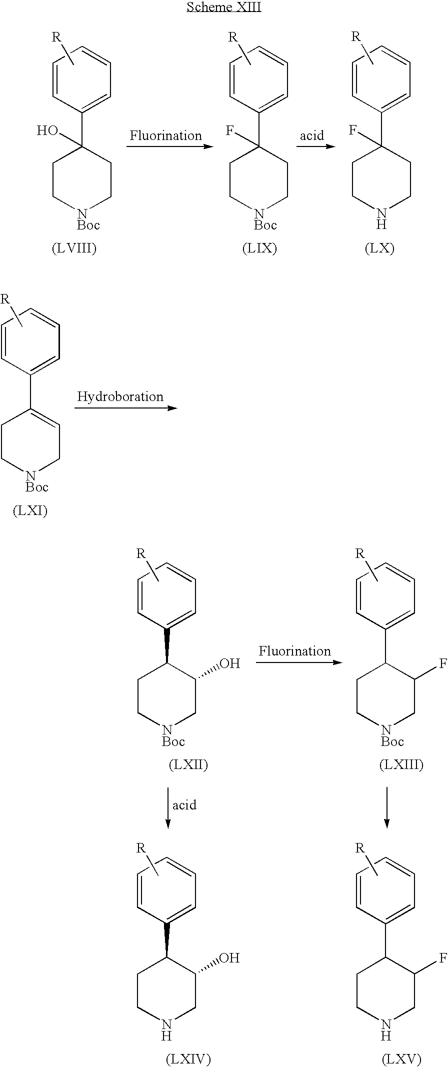

### WORKING EXAMPLES

The following illustrate the synthesis of various compounds of the present invention. Additional compounds within the scope of this invention may be prepared using the methods illustrated in these Examples, either alone or in combination with techniques generally known in the art.

Preparation of Substituted Benzimidazoles (III) or (IV):

### Preparation 1

### 5,6-Difluoro-1-methyl-1H-benzimidazole-2-carbaldehyde Hydrochloride Hydrate

Sodium methylate (54 g, 1 mol) was added to a solution of diethoxyacetonitrile (139 mL, 1 mol) in methanol (500 mL). The reaction mixture was kept at room temperature for 24 g, then the reaction mixture was evaporated, treated with water (500 mL), and the product was extracted with ether (2×300 mL). The combined organic extracts were dried over anhydrous K₂CO₃ and evaporated to give 114.62 g (60% purity) of methyl 2,2-diethoxyethanimidoate. The obtained crude product was used for the next stage without additional purification.

10% Pd/C (2 g) was added to a solution of 4,5-difluoro-2-nitroaniline (40 g, 0.229 mol in methanol (500 mL). The reaction mixture was hydrogenated at room temperature for 3 h and treated with the mixture of compound methyl 2,2-diethoxyethanimidoate (71 g, 0.23 mol) with acetic acid (60 g). After 16 h at room temperature, the reaction mixture was evaporated, washed with the 10% K₂CO₃ solution (1 L), and the product vas extracted with ether (300 mL). The organic extract was evaporated, and the residue was purified on silica gel (ethyl acetate/hexane 1:5) to give 48 g of 2-(diethoxymethyl)-5,6-difluoro-1H-benzimidazole.

Cs₂CO₃ (67.21 g, 0.206 mol) and methyl iodide (27 g, 0.19 mol) were added to a solution of 2-(Diethoxymethyl)-5,6-difluoro-1H-benzimidazole (48 g, 0.1875 mol) in DMF (250 mL). The reaction mixture was stirred at room temperature for 16 h and evaporated. Ethyl acetate (300 mL) was added to the residue, and the reaction mixture was washed with water (1 L). The organic layer was separated, dried, and evaporated. The residue was purified on silica gel (ethyl acetate/hexane 1:5) to give 40.8 g of 2-(diethoxymethyl)-5,6-difluoro-1-methyl-1H-benzimidazole.

2-(Diethoxymethyl)-5,6-difluoro-1-methyl-1H-benzimidazole was treated with 5 M HCl (300 mL), and the reaction mixture was kept at 60° C. for 6 h. Then the reaction mixture was evaporated to a volume of 100 mL and treated with acetone (200 mL). Then the mixture was kept in a refrigerator, and white crystals precipitated. The crystals were separated by filtration and dried under a reduced pressure to give 5,6-Difluoro-1-methyl-1H-benzimidazole-2-carbaldehyde Hydrochloride Hydrate in 72% (26.77 g, 21.4 g of free base) yield. 400 MHz ¹H NMR (D₂O) δ (ppm): 7.9 (m, 1H), 7.8 (m, 1H), 6.5 (s, 1H), 4.1 (s, 3H); MS⁺ 197

### Preparation 2

### 1-(Fluoromethyl)-1H-benzimidazole-2-carbaldehyde Hydrochloride Hydrate

Formaldehyde (37% solution in water, 315 mL, 4.208 mol) was added to a stirred solution of 2-methylbenzimidazole (97.0 g, 734 mmol) in MeOH (500 mL). The mixture was refluxed overnight. The formed precipitate was separated by filtration and dried in vacuo to afford (2-Methyl-1H-benzimidazol-1-yl)methanol (388.0 g 74%).

DAST (29.6 mL, 224 mmol) was added dropwise to a stirred solution of (2-Methyl-1H-benzimidazol-1-yl)methanol (33.0 g, 203 mmol) in CH₂Cl₂ (700 mL at −80° C. The mixture was stirred overnight at room temperature. The reaction mixture was poured into ice-cold water, and pH was adjusted to 9 with a 10% NaOH solution. The organic phase was separated, dried with Na₂SO₄, and concentrated to afford 1-(Fluoromethyl)-2-methyl-1H-benzimidazole (23.3 g, 70%).

Selenium dioxide (94.6 g, 853 mmol) was added to a stirred solution of 1-(Fluoromethyl-2-methyl-1H-benzimidazole (70.0 g 426 mmol) in dioxane (1.5 L), and the mixture was refluxed overnight. The solids were filtered off, and the filtrate was concentrated. The residue was purified by column chromatography (silica gel, CHCl₃/MeOH 15:1) and dried. The resulting product was dissolved in concentrated HCl (100 mL), and the solution was concentrated in vacuo to afford 1-(Fluoromethyl)-1H-benzimidazole-2-carbaldehyde Hydrochloride Hydrate (32.1 g, 32%). 400 MHz ¹H NMR (DMSO-d⁶+TFA) δ (ppm): 10.0 (s, 1H), 8.0 (m, 2H), 7.6 (m, 1H), 7.5 (m, 1H), 6.8 (s, 1H), 6.6 (s, 1H); MS⁺ 179.

### Preparation 4

### 1-(2-Methoxyethyl)-1H-benzimidazole-2-carbaldehyde Hydrochloride Hydrate

2-(Diethoxymethyl)-1H-benzimidazole (50 g, 0.227 mol) was suspended in DMF (200 mL), and Cs₂CO₃ (81.4 g, 0.227 mol) was added. 1-Bromo-2-methoxyethane (21.4 mL, 0.25 mol) was added dropwise, and the mixture was stirred overnight. Then the precipitate was filtered through Celite, and DMF was rotary evaporated. Water (300 mL) and diethyl ether (500 mL) were added to the residue. The organic layer was washed with water (2×300 mL), dried over Na₂SO₄, and ether was evaporated. The residue was the virtually pure 2-(Diethoxymethyl)-1-(2-methoxyethyl)-1H-benzimidazole obtained in 97% (61 g) yield.

2-(Diethoxymethyl)-1-(2-methoxyethyl)-1H-benzimidazole (61 g, 0.219 mol) was dissolved in 6 M aqueous HCl (150 mL). The solution was kept at 60-70° C. for 12 h. Most of water and acid was rotary evaporated, and the product crystallized from the residue. The product was filtered, washed with acetone and ether, and vacuum-dried to give 1-(2-methoxyethyl)-1H-benzimidazole-2-carbaldehyde Hydrochloride Hydrate in 64% (36.1 g) yield. 400 MHz ¹H NMR (D₂O) δ (ppm): 7.8-8.0 (m, 2H), 7.7 (m, 2H), 6.6 (s, br, 1H), 4.0 (m, br 4H), 3.4 (s, 3H); GC-MS 204.

Similar procedure was used to prepare.

1-(2-Fluoroethyl)-1H-benzimidazole-2-carbaldehyde Hydrochloride Hydrate. 400 MHz ¹H NMR (D₂O) δ (ppm): 7.8-8.0 (m, 2H), 7.7 (m, 2H), 6.6 (s, br, 1H), 5.1 (m, 2H), 5.0 (m, 2H); GC-MS 192.

### Preparation 5

### 1-(Methoxymethyl)-1H-benzimidazole-2-carbaldehyde

A mixture of tartaric acid (94.5 g, 630 mmol), o-phenylenediamine (163.5 g, 1512 mmol), water (158 mL), ethanol (194.5 mL), 12N hydrochloric acid (157.5 mL), and 85% phosphoric acid (63 mL) was kept at 135° C. for 12 h. The solid phase was filtered and dissolved in water, and charcoal was added. The mixture was refluxed for 2 h, filtered, and alkalized with ammonium hydroxide. The solid phase was filtered, washed with acetone, and dried to give 1,2-bis(1H-Benzimidazol-2-yl)ethane-1,2-diol in 51% (114 g) yield.

60% NaH in oil (29.4 g, 734 mmol) was added in portions to a stirred suspension of 1,2-bis(1H-Benzimidazol-2-yl)ethane-1,2-diol (108.0 g, 367 mmol) in DMF (2 L). The mixture was stirred for 1 h, and chloromethyl methyl ether (59.1 g, 734 mmol) was added dropwise, and the reaction mixture was stirred at room temperature overnight. The reaction mixture was concentrated in vacuum, poured into water (2 L), and HCl was added to pH 3. The mixture was extracted with chloroform (3×200 mL), and ammonium hydroxide was added to pH 10. The product was extracted with chloroform (4×200 mL). The extracts were combined, dried over sodium sulfate, and concentrated. The residue was purified by column chromatography (silica gel, CHCl₃/MeOH 25:1) and the resumed product was recrystallized from i-PrOH to give 1,2-bis[1-(Methoxymethyl)-1H-benzimidazol-2-yl]ethane-1,2-diol in 21% (28.9 g) yield.

NalO₄ (16.16 g, 76 mmol) was added to a solution of 1,2-bis[1-(Methoxymethyl)-1H-benzimidazol-2-yl]ethane-1,2-diol (28.9 g, 76 mmol) in 1 N H₂SO₄, (1 L), and the reaction mixture was stirred at room temperature overnight. The solution was neutralized with NaHCO₃, and the product was extracted with EtOAc (4×200 mL). The combined extracts were dried over sodium sulfate, concentrated, and poured into hexane. The solid phase was filtered, washed with hexane, and dried to give 1-(Methoxymethyl-1H-benzimidazole-2-carbaldehyde in 89% (25.6 g) yield. 40 MHz ¹H NMR (DMSO-d⁶) δ (ppm): 10.1 (s, 1H), 7.9 (d, 1H), 7.8 (d, 1H), 7.5 (t, 1H, 7.4 (t, 1H), 6.0 (s, 2H), 3.2 (s, 3H); GC-MS 190.

### Preparation 6

### 1-Cyclopropyl-1H-benzimidazole-2-carbaldehyde-Hydrochloride-Hydrate

1-Chloro-2-nitrobenzene (47.3 g, 0.300 mol) was dissolved in hexametapol (50 mL), and cyclopropylamine (51.4 g, 0.900 mol) was added. The mixture was boiled for 7 h. The 70% degree of conversion was attained according to ¹H NMR data. The reaction mixture was diluted with water (500 mL) and extracted with ether (2×200 mL). The ether layer was washed with water (2×300 mL). The organic layer was dried over Na₂SO₄. Ether was evaporated to give a mixture (53 g) containing 30% 1-chloro-2% nitrobenzene and N-Cyclopropyl-2-nitroaniline in 70% yield.

N-Cyclopropyl-2-nitroaniline (53.0 g, 0.297 mol) was dissolved in methanol (500 mL), and 10% Pd/C (2.5 g) was added in argon. The mixture was hydrogenated in a Parr apparatus at 20 psi for 1 h. The mixture self-heated, therefore hydrogen was supplied in portions so that the temperature was no higher than 50° C. After the reaction mixture was completely decolorized, the catalyst was filtered off, and acetic acid (60 mL) was added to the residue. This mixture containing N-Cyclopropylbenzene-1,2-diamine was used at the next step.

Methyl 2,2-Diethoxyethanimidoate (61.1 g, 0.446 mol) was added to the solution containing N-Cyclopropylbenzene-1,2-diamine and the mixture was kept at room temperature for 24 h. Then methanol was evaporated, and the residue was dissolved in ether (500 mL). The solution was washed with the 5% solution of Na₂CO₃ (2×300 mL), and ether was dried over Na₂SO₄. Ether was evaporated, and the residue was subjected to chromatography on silica gel (dichloromethane→dichloromethane/10% diethyl ether) to give 1-Cyclopropyl-2-(diethoxymethyl)-1H-benzimidazole in 28% (22 g) yield.

1-Cyclopropyl-2-(diethoxymethyl)-1H-benzimidazole (22.0 g, 0.0845 mL) was dissolved in 20% aqueous HCl (150 mL). The solution was heated at 60-70° C. for 4 h. The main portion of water and acid was rotary evaporated. Acetone (200 mL) and ether (50 mL) was added to the residue. The precipitate was separated by filtration, washed with acetone and ether, and vacuum-dried to give 1-Cyclopropyl-1H-benzimidazole-2-carbaldehyde Hydrochloride Hydrate in 96% (19.7 g) yield. 400 MHz ¹H NMR (D₂O) δ (ppm): 8.0 (m, 1H), 7.8 (m, 1H), 7.6 (m, 2H), 6.6 (s, br, 1H), 3.7 (m, 1H), 1.4 (m 2H), 1.3 (m, 2H); GC-MS 186.

### Preparation 7

### 2-Formyl-1-methyl-1H-benzoimidazole-5-carbonitrile

4-Chloro-3-nitro-benzonitrile (30 g, 165 mmol) was suspended in EtOH (60 mL), and methylamine (33% in EtOH, 24 ml, 165 mmol) was added. The mixture was stirred at room temperature for 1 h, then heated to 70° C. overnight. The reaction mixture was cooled to rt and concentrated in vacuo. The residue was suspended in Et₂O and filtered to give 42 g of 4-methylamino-3-nitro-benzonitrile, which was used in the next step without further purification. 400 MHz ¹H NMR (CD₃OD) δ (ppm): 8.5 (s, 1H), 7.70 (d, J=9 Hz, 1H), 7.09 (d, J=9 Hz, 1H), 3.29 (s, 3H), APCl MS⁺ 177.

4-Methylamino-3-nitro-benzonitrile (42 g, 0.297 mol) was suspended in EtOH/H₂O (10:1, 1000 mL) under N₂ at rt. Iron powder (53 g, 948 mmol) and CaCl₂ (24 g, 216 mmol) were added and the mixture was heated at reflux for 2 hours. TLC showed no starting material was left. The mixture was cooled to room temperature and filtered through a pad of celite. The filtrate was concentrated in vacuo and the residue was re-dissolved in CH₂Cl₂ (400 mL) and washed with water, brine and dried over sodium sulfate. The solvent was removed in vacuo to give 20.5 g of the desired product, 3-amino-4-methylamino-benzonitrile, 400 MHz ¹H NMR (CD₃OD) δ (ppm): 7.02 (d, J=8 Hz, 1H), 6.86 (s, 1H) 6.51 (d, J=8 Hz, 1H), 3.28 (s, br, 3H); MS⁺ 148.

Methyl 2,2-Diethoxyethanimidoate (56.2 g, 349 mmol, and acetic acid (24 mL, 420 mmol) were added to the solution of 3-amino-4-methylamino-benzonitrile in MeOH and the mixture was kept at room temperature for 3 hours. The mixture was concentrated in vacuo and the residue was dissolved in EtOAc (500 mL). The solution was washed with the 5% solution of Na₂CO₃ (2×300 mL), and dried over Na₂SO₄. EtOAc was evaporated, and the residue was subjected to chromatography on silica gel (3:1 hexane:EtOAc) to give 35 g of 2-Diethoxymethyl-1-methyl-1H-benzoimidazole-5-carbonitrile. 400 MHz ¹H NMR (CD₃OD) δ (ppm): 8.04 (s, 1H), 7.77 (d, J=9 Hz, 1H), 7.64 (d, J=9 Hz, 1H), 5.74 (s, 1H), 3.97 (s, 3H), 3.75-3.83 (m, 2H), 3.59-3.66 (m, 2H), 1.23 (m, 6H); MS⁺ 260.

2-Diethoxymethyl-1-methyl-1H-benzoimidazole-5-carbonitrile (35 g, 135 mmol) was dissolved in 4N HCl in dioxane (135 mL) and the solution was heated at 60° C. for 8 h. The mixture was concentrated in vacuo and the residue was suspended in Et₂O (200 mL). The precipitate was separated by filtration, washed with hexane, and vacuum-dried to give 25 g of 2-Formyl-1-methyl-1H-benzoimidazole-5-carbonitrile Hydrochloride Hydrate as a tan solid. MS⁺ 186.

Similar procedure was used to prepare:

4-Bromo-1-methyl-1H-benzoimidazole-2-carbaldehyde from 1-Bromo-3-fluoro-2-nitro-benzene; MS⁺ 239, 241.

5-Bromo-1-methyl-1H-benzoimidazole-2-carbaldehyde from 4-Bromo-1-fluoro-2-nitro-benzene; MS⁺ 239, 241.

5-Fluoro-1-methyl-1H-benzoimidazole-2-carbaldehyde from 1,4-Difluoro-2-nitro-benzene; MS⁺ 179

5-Trifluoromethyl-1-methyl-1H-benzoimidazole-2-carbaldehyde from 1-Chloro-2-nitro-4-trifluoromethyl-benzene; MS⁺ 229

5-Chloro-1-methyl-1H-benzoimidazole-2-carbaldehyde from 1,4-Dichloro-2-nitro-benzene. MS⁺ 195

2-Formyl-1-methyl-1H-benzoimidazole-6-carbonitrile from 3-chloro-4-nitrobenzonitrile. MS⁺ 186

### Preparation 8

### 6-Bromo-1-methyl-1H-benzo[d]imidazole-2-carbaldehyde

To a solution of hydroxy peroxide (30% in H₂O, 46.5 mL, 480 mmol) under N₂ at 0° C. trifluoroacetic anhydride (75.8 mL 550 mmol) in 160 mL dichloromethane was added drop wisely over one and half hour. After addition, 4-bromo-2-fluorobenzenamine (10 g, 52.6 mmol) in 160 mL dichloromethane was added dropwise and the reaction mixture was gradually warmed up to room temperature and stirred over night. The mixture was extracted by dichloromethane and dried over anhydrous Na₂SO₄. The mixture was filtered and concentrated under reduced pressure to yield 4-bromo-2-fluoro-1-nitrobenzene (10.59 g). 400 MHz ¹H NMR (CDCl₃) δ 8.0 (t, 1H) 7.4-7.6 (m, 2H); MS⁺ 219, 221.

To a solution of 4-bromo-2-fluoro-1-nitrobenzene (10 g, 45.45 mmol) in 20 ml of ethanol under 0° C., 30 ml methylamine (33 wt % in ethanol) was added dropwise. After addition, reaction mixture was gradually warmed up to room temperature and stirred for 25 minutes. The mixture was concentrated under reduced pressure to yield N-methyl-5-bromo-2-nitrophenyl)amine (9.13 g). 400 MHz ¹H NMR (CDCl₃) δ 8.0 (d, 1H), 7.0 (d, 1H), 6.7 (dd, 1H). MS⁺ 230, 232.

To a solution of 120 ml ethanol and 30 ml water, N-methyl-(5-bromo-2-nitrobenzene)amine (10.5 g, 45.4 mmol), iron powder (11.4 g, 204.5 mmol) and calcium chloride (4.54 g, 40.9 mmol) was added sequentially. The mixture was heated to reflux for 2 hours. After cooling down to room temperature, the mixture was filtered over celite and concentrated under reduced pressure to yield 5-bromo-N¹-methylbenzene-1,2-diamine (9.13 g). MS (M+1) 201, 203.

To a solution of 42 mL of 6N HCl and 63 mL of water, 5-bromo-N¹-methylbenzene 1,2-diamine (9.1 g, 45.4 mmol) and glycolic acid (17.2 g, 227.2 mmol) was added sequentially. The mixture was heated to reflux for 2 hours. After cooling down to room temperature, the mixture was neutralized to PH 9 by ammonium hydroxide. Precipitate formed filtered, rinsed by water and dried by vacuum to yield (6-bromo-1-methyl-1H-benzo[d]imidazol-2-yl)methanol (8.5 g). 400 MHz ¹H NMR (CDCl₃) δ 7.5 (d, 1H), 7.46 (m, 1H), 7.35 (dd, 1H), 4.92 (s, 2H), 4.74 (b, 1H), 3.8 (s, 3H); MS (M+1) 241, 243.

To a solution of 8 ml dichloromethane, (6-bromo-1-methyl-1H-benzo[d]imidazol-2-yl)methanol (1.0 g, 4.1 mmol) and manganese(IV) oxide activated, 1.8 g, 20.7 mmol) was added sequentially. The mixture was irradiated by microwave under 90° C. for 30 minutes. After cooling down to room temperature, the mixture was filtered over a short pad of silica gel and concentrated under reduced pressure to yield 6-bromo-1-methyl-1H-benzo[d]imidazole-2-carbaldehyde (0.64 g). 400 MHz ¹H NMR (CDCl₃) δ 10.5 (s, 1H), 7.8 (d, 1H), 7.6 (s, 1H), 7.5 (d, 1H), 4.1 (s, 3H); MS (M+1) 239, 241.

### Preparation 9

### 7-Bromo-1-methyl-1H-benzo[d]imidazole-2-carbaldehyde

To a stirred solution of 2-chloro-3-nitrobenzoic acid (15 g, 74.4 mmol) in 350 mL of CCl₄ under N₂ at room temperature was added red HgO (24.3 g, 111.892 mmol). The mixture was irradiated by light and heated to reflux at 87° C. and Br₂ (5.75 mL, 111.6 mmol) was added dropwise. After the addition was complete, the mixture was refluxed for additional 3 h and then cooled to room temperature. The reaction was quenched with saturated NaHCO₃ aqueous solution. The mixture was stirred for 10 min and filtered through a pad of celite. The cake was further rinsed with CH₂Cl₂. The organic layer was separated and washed with water, brine and dried over Na₂SO₄. The solvent was removed in vacuo to give 11.55 g of 1-bromo-2-chloro-3-nitrobenzene (87%). 400 MHz ¹H NMR (CDCl₃) δ 7.84 (d, 1H), 7.71 (d, 1H), 7.27 (dd, 1H); MS (M+1) 235, 237, 239.

To a stirred solution of 1-bromo-2-chloro-3-nitrobenzene in 50 mL of ethanol was added methyl amine solution (40% in methanol, 50 mL) at room temperature. The mixture was then heated to 85° C. under N₂ for 1 h. LC-MS showed small amount of starting material. Additional methylamine solution (10 mL) was added and the reaction mixture was heated for another hour and then cooled to room temperature. The mixture was extracted with CHₑCl₂ and the organic layer was washed with water, brine and dried with Na₂SO₄. The solvent was removed in vacuo to give 4.5 g of 2-bromo-N-methyl-6-nitrobenzenamine (92%). 400 MHz ¹H NMR (CDCl₂) δ 7.84 (d, 1H), 7.65 (d, 1H), 6.65 (dd, 1H), 3.0 (s, 3H), MS (M+1) 231, 233, 234.

2-Bromo-N-methyl-6-nitrobenzenamine (2.42 g, 10.5 mmol), iron powder (2.93 g, 52.5 mmol) were mixed in 45 mL of 3N HCl aqueous solution. The mixture was heated to reflux at 105° C. and the reaction vas monitored by LC-MS. After 30 min, LC-MS showed a single peak with correct mass of the desired dianiline product. Glycolic acid (8.0 g, 105 mmol) was added and the reaction mixture was stirred at reflux for additional 1 h. The reaction mixture was cooled to room temperature and filtered through a pad of celite. The filtrate was adjusted to PH=9 using aqueous NH₄OH to form brownish precipitate. The solid was collected through filtration, washed by water and dried in vacuo to give 3.18 g of (7-bromo-1-methyl-1H-benzo[d]imidazol-2-yl)methanol with some water, 400 MHz ¹H NMR (DMSO-d⁶) δ (ppm): 7.56 (d, 1H), 7.38 (d, 1H), 7.06 (t, 1H), 5.52 (br, 1H), 4.54 (s, 2H), 4.05 (s, 3H); MS⁺ 241, 243.

(7-Bromo-1-methyl-1H-benzo[d]imidazol-2-yl)methanol (368 mg, 1.5 mmol), activated MnO₂ were mixed in CH₂Cl₂ in a microwave tube and the reaction mixture was heated in a microwave reactor at 70° C. for 20 min. The reaction mixture was then filtered through a pad of celite and the cake was further rinsed with CH₂Cl₂. The filtrate was concentrated to give 161 mg of 7-bromo-1-methyl-1H-benzo[d]imidazole-2-carbaldehyde (45%), 400 MHz ¹H NMR (CDCl₃) δ (ppm): 10.1 (s, 1H), 7.85 (m, 1H), 7.60 (m, 1H) 7.24 (t, 1H), 4.50 (s, 3H); MS⁺ 239 241.

### Preparation 10

### 7-Fluoro-1-methyl-1H-benzo[d]imidazole-2-carbaldehyde

To a stirred solution of hydroxy peroxide (30% in H₂O, 35 mL, 356 mmol) under N₂ at 0° C., a solution of trifluoroacetic anhydride (56 mL, 407 mmol) in 115 mL dichloromethane was added dropwise over 2 h. After addition was complete, a solution of 2,3-difluorobenzenamine (5 g, 39 mmol) in 115 mL dichloromethane was added dropwise at 0° C. After the addition was complete, the reaction mixture was gradually warmed up to room temperature and stirred overnight. The mixture was extracted by dichloromethane and dried over anhydrous Na₂SO₄. The mixture was filtered and concentrated under reduced pressure. The residue was purified by flash column with 0-15% EtOAc in hexane to yield 3.5 g of 1,2-difluoro-3-nitrobenzene (57%). 400 MHz ¹H NMR (CCCl₃) δ (ppm) 7.84 (m, 1H), 7.50 (m, 1H), 7.26 (m, 1H).

To a stirred solution of 1,2-difluoro-3-nitrobenzene (1.74 g, 11 mmol) in 2 mL of ethanol was added methyl amine solution (33% in methanol, 1.4 mL, 11 mmol). The mixture was then heated in a microwave reactor at 70° C. for 10 min. LC-MS showed small amount of starting material. Additional methylamine solution (0.7 mL) was added and the reaction mixture was heated in the microwave reactor for another 20 min at 70° C. The mixture was extracted with CHₑCl₂ and the organic layer was washed with water, brine and dried over Na₂SO₄. The solvent was removed in vacuo to give 2.6 g of 2-fluoro-N-methyl-6-nitrobenzenamine. MS (M+1) 171.

2-Fluoro-N-methyl-6-nitrobenzenamine (2.7 g, 15.8 mmol), iron powder (4.4 g, 79 mmol; were mixed in 30 mL H₂O and 11 mL of 6N HCl aqueous solution was added. The mixture was heated to reflux and the reaction was monitored by LC-MS. After all starting material was consumed, the reaction mixture was cooled to room temperature and filtered through a pad of celite. The filtrate was adjusted to PH=9 using 6N NaOH and the mixture was extracted with CH₂Cl₂ and the organic layer was washed with water, brine and dried over Na₂SO₄. The solvent was removed in vacuo to give 1.76 g of 6-fluoro-N¹-methylbenzene-1,2-diamine. 400 MHz ¹H NMR (CDCl₃) δ (ppm) 6.76 (m, 1H), 6.44 (m, 2H), 2.70 (s, 3K), MS⁺ 141.

6-Fluoro-N¹-methylbenzene-1,2-diamine (1.56 g, 11.1 mmol), triethylorthoacetate (2.85 mL, 15.6 mmol) were mixed in ethanol (3 mL). The reaction mixture was heated in a microwave reactor at 170° C. for 30 min. The reaction mixture was cooled to room temperature and concentrated in vacuo. The residue was cooled in freezer for 2 h and the precipitates were collected through filtration and washed with cole hexane to give 966 mg of 7-fluoro-1,2-dimethyl-1H-benzo[d]imidazole (53%). ¹H NMR (CDCl₃) δ (ppm) 7.4 (d, 1H), 7.08 (m, 1H), 6.88 (dd, 1H), 3.90 (s, 3H), 2.56 (s, 3H), MS⁺ 165.

7-Fluoro-1,2-dimethyl-1H-benzo[d]imidazole (400 mg, 2.44 mmol), SeO₂ (270 mg, 2.44 mmol) were mixed in 6 mL of 1,4-dioxane. The mixture was heated in a microwave reactor at 150° C. for 20 min. Some starting material was still left. The mixture was heated at 170° C. for 20 min and all starting material was consumed. The reaction mixture was filtered through a pad of celite. The filtrate was concentrated and the residue was purified by faish column with 0-40% EtOAc in hexane to give 198 mg of 7-fluoro-1-methyl-1H-benzo[d]imidazole-2-carbaldehyde. ¹H NMR (CDCl₃) δ (ppm) 10.1 (s, 1H), 7.7 (d, 1H), 7.25 (m, 1H), 7.1 (dd, 1H), 4.38 (s, 3H); MS⁺ 178.

### Preparation 11

### 2-(Chloromethyl)-1-methyl-1H-benzo[d]imidazole-5-carbonitrile

To a stirred solution of 3-amino-4-methylamino-benzonitrile (530 mg, 3.6 mmol), synthesized as in preparation 7, in 6N HCl aqueous solution (10 mL) was added chloroacetic acid (511 mg, 5.4 mmol). The mixture was then stirred at 100° C. for 4 hours. The mixture was then cooled to room temperature and neutralized with aqueous NH₄OH. A beige precipitate formed upon cooling the mixture in an ice bath. The solid was collected by filtration and dried under high vacuo to give 2-(Chloromethyl)-1-methyl-1H-benzo[d]imidazole-carbonitrile (396 mg, 55%). 400 MHz ¹H NMR (CDCl₂) δ (ppm): 8.07 (s, 1H), 7.59 (d, J=8 Hz, 1H), 7.44 (d, J=8 Hz, 1H), 4.86 (s, 2H), 3.92 (s, 3H), MS⁺ 206, 208.

### Preparation 12

### 2-(Chloromethyl)-4-fluoro-1-methyl-1H-benzo[d]imidazole

To a solution of tert-butyl 2-fluoro-6-nitrophenylcarbamate (5.77 g, 22.5 mmol) in ethanol (50 mL) was added Pd/C (10%, 600 mg). The mixture was then shaked under H₂ (35 PSI) for 3 h. TLC showed no starting material was left. The reaction mixture was filtered through a pad of celite and the filtrate was concentrated under vacuo. The residue was azeotroped with toluene (2×) to give tert-butyl 2-amino-fluorophenylcarbamate as a grey solid (5.10 g). 400 MHz ¹H NMR (CD₃OD) δ (ppm): 6.94 (dd, J=8 Hz, 6 Hz, 1H), 6.54 (d, J=8 Hz, 1H), 6.40 (t, J=6 Hz, 1H), 1.48 (s, 9H).

tert-Butyl 2-amino-6-fluorophenylcarbamate (5.1 g, 22.5 mmol), formaldehyde (1.68 mL, 22.5 mmol) and Pd/C (10%, 500 mg) were mixed together in 30 mL ethanol. The mixture was then shaked under H₂ (40 PSI) overnight at room temperature. The mixture was filtered through a pad of celite and the cake was further washed with ethanol. The filtrate was concentrated in vacuo to give tert-butyl 2-fluoro-6-(methylamino)phenylcarbamate as a grey solid (5.7 g). 400 MHz ¹H NMR (CD₃OD) δ (ppm): 7.05 (m, 1H), 6.36-6.42 (m, 2H), 2.79 (s, 3H), 1.15 (m, 9H).

To a stirred solution of tertbutyl 2-fluoro-6-(methylamino)phenylcarbamate (5.7 g) in methylene chloride (50 mL) at room temperature vas added a solution of 4N HCl in dioxane (100 mL). The mixture was stirred at room temperature for 5 hours and the reaction mixture was partitioned between 1N NaOH aq. and methylene chloride. The aqueous layer was separated and further extracted with methylene chloride. The organic layers were combined and dried over Na₂SO₄. Filtered and the solvent was removed in vacuo to give 3-fluoro-N¹-methylbenzene-1,2-diamine as a yellowish oil (2.7 g). 400 MHz ¹H NMR (CD₃OD) δ (ppm): 6.63 (m 1H), 6.40 (m, 2H), 2.80 (s, 3H).

To a stirred solution of 3-fluoro-N¹-methylbenzene-1,2-diamine (2.7 g, 19.3 mmol) in 6N HCl aqueous solution (10 mL) was added chloroacetic acid (2.73 g, 28.9 mmol). The mixture was stirred at 100° C. for 5 hours and was then cooled to room temperature. The mixture was neutralized by aqueous NH₄OH solution. The resulted gummy precipitate was collected by filtration and further purified via flash column (silica gel, hexane:EtOAc 1:1) to give 2-(Chloromethyl)-4-fluoro-1-methyl-1H-benzo[d]imidazole (1.45 g). 400 MHz ¹H NMR (CDCl₃) δ (ppm) 7.26-7.35 (m, 2H), 6.98 (m, 1H), 4.88 (s, 2H), 3.90 (s, 3H); MS⁺ 199, 201.

### Preparation Methods of Substituted Piperidine Templates (II)

For each preparation method, a representative synthesis is described. Other templates prepared through a similar synthetic sequence are listed in tables followed the description.

### Method A

### Scheme IV

### Triflate Coupling/Hydrogenation

### Preparation 13

### 4-(4-Fluorophenyl)pyperidine hydrochloride

To a stirred solution of diisopropylamine (7 ml) in THF (150 ml) at −78° C. was added a solution of n-butyl lithium in hexanes (20 ml, 2.5 M). After 1 h tert-butyl 4-oxo-1-piperidinecarboxylate (10 g) was added. After an additional 1.5 h N-phenyltrifluoromethanesulfonimide (19.65 g) was added and the mixture was allowed to warm to room temperature. After stirring for 16 h the solvent was removed under reduced pressure and the resulting residue was used in the next step without purification.

A mixture of tert-butyl 4-trifluoromethanesulfonate-1-(1,2,3,6-tetrahydropyridine) carboxylate (8.3 g, crude), 4-fluorophenylboronic acid (3.5 g) and tetrakis(triphenylphosphine)palladium(0) (2.89 g) in a mixture of ethanol (85 ml and water (15 ml) was stirred at 90° C. After 16 h the solvents were removed under reduced pressure, water was added and the mixture was extracted with ethyl acetate. The combined organics were washed with brine, dried over sodium sulfate, filtered and concentrated under reduced pressure. Purification by flash chromatography using a silica gel column and eluting with a gradient of 0% to 20% ethylacetate in hexanes gave 3.2 g of tert-butyl 4-(4-fluorophenyl)-1-(1,2,3,6-tetrahydropyridine) carboxylate as a brownish oil: ¹H NMR (400 MHz, CD3OD) δ 1.47 (s, 9H), 2.49 (m, 2H), 3.61 (m, 2H), 4.03 (m, 2H), 6.04 (m, 1H), 7.04 (t, 1H), 7.23-7.44 (m, 3H).

A mixture of tert-butyl 4-(4-fluorophenyl-1-(1,2,3,6-tetrahyropyridine) carboxylate (3.2 g) and 10% Pd on carbon (60 mg) in ethanol (20 ml) was shaken in a Parr apparatus under 40 psi of hydrogen. After 16 h the mixture was purged with nitrogen, filtered through celite, and concentrated under reduced pressure to give 3.2 g of tert-butyl 4-(4-fluorophenyl)-1-piperidinecarboxylate as a yellow oil: ¹H NMR (400 MHz, CD3OD) δ 1.47 (s, 9H), 1.49-1.62 (m, 2H), 1.79 (d, 2H), 2.66-2.74 (m, 1H), 2.85 (m, 2H), 4.18 (m, 2H), 6.99 (t, 1H), 7.18-7.38 (m, 3H).

A solution of tert-butyl 4-(4-fluorophenyl)-1-piperidinecarboxylate (3.2 g) in 4M HCl/dioxane (10 ml) was stirred at room temperature. After 2 h the mixture was concentrated under reduced pressure to give 2.5 g of 4-(4-fluorophenyl)pyperidine hydrochloride as a white solid, ¹H NMR (400 MHz, CD3OD) δ 1.82-1.93 (m, 2H), 2.03-2.08 (m, 2H), 2.88-2.94 (m, 1H), 3.09-3.16 (m, 2H), 3.47-3.50 (m, 2H), 7.05 (t, 1H), 7.23-7.39 (m, 3H); MS (m/z) 180.1.

The following 4-substituted piperidines were prepared as above starting with the reaction of tert-butyl 4-trifluoromethanesulfonate-1-(1,2,3,6-tetrahydropyridine) carboxylate and the appropriate arylboronic acid:

TABLE 1

| NAME | m/z |
|---|---|
| 4-(5-fluoro-2-methoxyphenyl)piperidine hydrochloride | 210.1 |
| 4-(3,5-dimethylphenyl)piperidine hydrochloride | 190.2 |
| 4-(2-(1-hydroxyethyl)phenyl)piperidine hydrochloride | 206.2 |
| 4-(3-methylphenyl)piperidine hydrochloride | 176.2 |
| 4-(4-trifluoromethoxyphenyl)piperidine hydrochloride | 246.1 |
| 4-(3,4-dimethylphenyl)piperidine hydrochloride | |
| 4-(2,5-dimethylphenyl)piperidine hydrochloride | 190.1 |
| 4-(3,5-di(trifluoromethyl)phenyl)piperidine hydrochloride | 298.3 |
| 4-(2,4-difluorophenyl)piperidine hydrochloride | 198.2 |
| 4-(4-fluoro-2-methylphenyl)piperidine hydrochloride | 194.2 |
| 4-(2-methylsulfonylphenyl)piperidine hydrochloride | |
| 4-(4-isopropoxyphenyl)piperidine hydrochloride | |
| 4-(2-trifluoromethoxyphenyl)piperidine hydrochloride | 246.3 |
| 4-(4-ethylphenyl)piperidine hydrochloride | |
| 4-(3-fluoro-4-methoxyphenyl)piperidine hydrochloride | |
| 4-(3,5-difluorophenyl)piperidine hydrochloride | 198.2 |
| 4-(2-fluoro-6-methoxyphenyl)piperidine hydrochloride | |
| 4-(4-ethoxyphenyl)piperidine hydrochloride | 206.2 |
| 4-(4-methylphenyl)piperidine hydrochloride | 176.2 |
| 4-(2,3-difluorophenyl)piperidine hydrochloride | 198.2 |
| 4-(4-fluoro-2-methoxyphenyl)piperidine hydrochloride | 210.1 |

### Preparation 14

### 4-(2-(1-hydroxyethyl)phenyl)piperidine hydrochloride

4-(2-(1-Hydroxyethyl)phenyl)piperidine hydrochloride was prepared as above starting with the reaction of tert-butyl 4-trifluoromethanesulfonate-1-(1,2,3,6-tetrahydropyridine) carboxylate and 2-acetylphenylboronic acid. Note that the acetyl group was reduced to its corresponding alcohol during the hydrogenation step. MS m/z 206.2

### Method B

### Scheme IV

### Triflate Coupling/PtO₂-Hydrogenation

### Preparation 15

### 4-(4-Chloro-3-fluorophenyl)piperidine hydrochloride

tert-Butyl 4-(4-chloro-3-fluorophenyl)-1-(1,2,3,6-tetrahydropyridine) carboxylate vas prepared following the first two steps of Preparation 13 using 4-chloro-3-fluorophenyl boronic acid. A mixture of tert-butyl 4-(3-chloro-4-fluorophenyl)-1-(1,2,3,6-tetrahydropyridine) carboxylate (465 mg, 1.49 mmol) and PtO₂ (20 mg) in methanol (8 ml) was shaken in a Parr apparatus under 45 psi of hydrogen. After 1 h the mixture was purged with nitrogen, filtered through celite, and concentrated under reduced pressure to give 443 mg of tert-butyl 4-(3-chloro-4-fluorophenyl)-1-pipendinecarboxylate as a yellow oil.

A solution of tert-butyl 4-(4-Chloro-3-fluorophenyl)-1-piperidinecarboxylate (886 mg) in 4N HCl/dioxane (3 ml) was stirred at room temperature. After 4 h the mixture was concentrated under reduced pressure to give 749 mg of 4-(4-chloro-3-fluorophenyl)pyperidine hydrochloride, MS (m/z+CH₃CN) 255, 257.

The following 4-substituted piperidines were prepared as above starting with the reaction of tert-butyl 4-trifluoromethanesulfonate-1-(1,2,3,6-tetrahydropyridine) carboxylate and the appropriate arylboronic acid:

TABLE 2

| NAME | m/z |
|---|---|
| 4-(3-Chloro-4-fluoro-phenyl)piperidine hydrochloride | 214.2 |
| 4-(2,4-difluorophenyl)piperidine hydrochloride | 198.2 |
| 4-(4-Chloro-2-fluoro-phenyl)piperidine hydrochloride | 214.2 |
| 4-(2-Chloro-4-fluorophenyl)piperidine hydrochloride | 214.2 |

### Method C

### Scheme V

### Organolithium/Grignard Addition

### Preparation 16

### 4-(4-trifluoromethylphenyl)piperidine Hydrochloride

A solution of 1-bromo-4-(trifluoromethyl)benzene (238.5 g, 1.06 mol) in anhydrous THF (500 mL) was added dropwise to a stirred solution of n-butyllithium (508 mL of a 2.5 M solution in hexanes, 1.27 mol) in anhydrous tetrahydrofuran (1.0 L) at 60° C. under an atmosphere of argon. The resultant reaction mixture was stirred at −60° C. for 1 h and then a solution of 1-benzylpiperidin-4-one in anhydrous tetrahydrofuran (600 mL) was added dropwise. The reaction mixture was allowed to warm to 0° C. and was stirred at this temperature for 2 h before being made acidic with the addition of concentrated hydrochloric acid. The two layers were separated and the aqueous layer was basified with concentrated ammonium hydroxide and extracted with diethyl ether (2>500 mL). The organic fraction was then dried (MgSO₄) and concentrated under reduced pressure to a thick slurry, and the resultant solid was filtered, washed with hexane, and air-dried to afford 1-benzyl-4-[4-(trifluoromethyl)phenyl]piperidin-4-ol (265 g, 75%); R_{f} 0.04 (20% ethyl acetate in hexane).

A solution of 1-benzyl-4-[4-trifluoromethyl)phenyl]piperidin-4-ol (123.5 g 0.37 mmol) in trifluoroacetic acid (750 ml) was heated at reflux over the weekend. The reaction mixture was then cooled to room temperature and concentrated under reduced pressure. Dichloromethane (1.0 L) and water (250 mL) were added to the residue and the pH of the solution was adjusted to 9 with the addition of concentrated ammonium hydroxide. The mixture was stirred at room temperature for 1 h, the organic phase was separated, and the aqueous phase was further extracted with dichloromethane (250 mL). The combined organic fractions were washed with water (250 mL), dried (MgSO₄), and the solvent was removed under reduced pressure to afford 1-benzyl-4-[4-(4-(trifluoromethyl)phenyl]-1,2,3,6-tetrahydropyridine (115.5 g, 98%) as an oil that solidified upon standing to give a granular beige solid; R_{f} 0.60 (7:8 ethyl acetate/hexane).

A solution of 1-benzyl-4-[4-trifluoromethyl)phenyl]-1,2,3,6-tetrahydropyridine (100 g, 0.315 mol) in methanol (600 mL) was treated with palladium on carbon (10.0 g), and hydrogen gas (40 atm) in an autoclave at 80° C. for 1 h. After being allowed to cool to room temperature, the reaction mixture was filtered through a pad of celite and concentrated under reduced pressure to half its volume. The residue was then acidified with concentrated hydrochloric acid (50 mL) and the remainder of the solvent was removed under reduced pressure to afford 4-[4-(trifluoromethyl)phenyl]piperidine hydrochloride (59.0 g, 69%) as an off-white solid, m.p. 196-197° C.; R_{f} 0.06 (75% ethyl acetate in methanol).

### Preparation 17

### 4-(5-Chloro-2-methoxyphenyl)piperidine

To a solution of 2-bromo-4-chloroanisole (164 g, 0.74 mol) in absolute THF (1 L) was added 2.7 M BuLi/heptane (280 mL) under stirring in an atmosphere of argon at −80° C. over a period of 1 h. The mixture was stirred for 30 min then was added a solution of N-Boc-4-piperidone (145 g, 0.73 mol) in absolute THF (250 mL) at −90° C. over a period of 1 h. The temperature was increased to −40° C. during 2 h, and were added 5M NaHSO₄ (160 mL), Na₂SO₄ (300 g), hexane (500 mL), and the mixture was stirred for 10 h. The organic layer was decanted, filtered through silica gel (300 g, 63/100 μm). The residue and silica gel were washed with 40% ethyl acetate/hexane (2×400 mL). The filtrate was evaporated to dryness, the residue was crystallized from a mixture ethyl acetate/hexane to afford tert-butyl 4-(5-Chloro-2-methoxyphenyl)-4-hydroxypiperidine-1-carboxylate in 39% (100 g, 0.29 mol) as white crystals.

To a solution of tert-butyl 4-(5-Chloro-2-methoxyphenyl)-4-hydroxypiperidine-1-carboxylate (90 g, 0.263 mol) in absolute dioxane (200 mL), was added 4 N HCl/dioxane (150 mL, 0.6 mol) under argon. The mixture was stirred for 24 h, evaporated, was added ether, and the evaporation was repeated. To the residue were added water (300 mL) and ether (500 mL). To the obtained mixture wt % as added Na₂CO₃ (32 g, 0.3 mol) under vigorous stirring, then was added CbzCl (43 mL, 0.3 mol) dropwise under cooling with an ice bath. The bath was removed, and the mixture was stirred for 1 h more. The layers were separated, the aqueous one was extracted with ether (2×200 mL). The combined organic layers were washed with water (200 mL), brine (200 mL), dried with Na₂SO₄, filtered through silica gel (100 g, 40/63 μm), and evaporated. Then was added absolute dioxane, and the evaporation was repeated. To a solution of the residue in absolute dichloromethane (300 mL) were added Et₃SiH (132 mL, 0.828 mmol), and TFA (96 mL, 1.24 mol) under argon. The mixture was stirred for 20 h and evaporated. To the residue were added a saturated K₄CO₃ solution to pH 10, water (˜200 mL), and the mixture was extracted with ether. The organic fractions were washed with water (2×200 mL), brine (200 mL) dried with Na₂SO₄, filtered through silica gel (100 g, 40/63 μm), and evaporated. To the residue was added absolute dioxane, and the evaporation was repeated. To a solution of the residue in absolute THF (300 mL) was added 1M BH₃/THF (260 mL) under cooling with an ice bath in an atmosphere of argon. The mixture was stirred at room temperature for 2 h, then was added AcOH (260 mL) under cooling with an ice bath in argon. The mixture was stirred for 24 h, evaporated, to the residue were added a saturated K₂CO₃ solution to pH 10, water (˜200 mL), and the mixture was extracted with ether. The organic fractions were washed with water (2×200 mL), brine (200 mL), dried with Na₂SO₄, and evaporated. The residue was purified on silica gel (500 g, 60/100 μm) with gradient elution from CCl₄ to CCl₄/EtOAc (10:1) to give benzyl 4-(5-Chloro-2-methoxyphenyl)piperidine-1-carboxylate in 77% (73 g) yield as a yellow oil.

To benzyl 4-(5-Chloro-2-methoxyphenyl)piperidine-1-carboxylate (73 g, 0.2 mol) was added concentrated HCl (200 mL). The mixture was refluxed at stirring for 2 h and evaporated to dryness. To the residue were added water (100 mL) and 10N NaOH (20 mL), the mixture was extracted with chloroform (3×200 ml). The organic layers were washed with water (200 mL), brine (200 mL), dried with Na₄SO₄, filtered through silica gel (100 g, 40/63 μm), and evaporated to afford 4-(5-Chloro-2-methoxyphenyl)piperidine in 89% (40 g) yield as white crystals.

The following 4-substituted piperidines were prepared through similar procedure to that described above featuring an addition of an organolithium or Grignard species to N-protected piperidin-4-one:

TABLE 3

| NAME | m/z |
|---|---|
| 3,3-Dimethyl-4-(4-(trifluoromethyl)phenyl)piperidine | 258 |
| 4-(5-Chloro-2-fluorophenyl)piperidine hydrochloride | 214 |
| 4-(2-Fluorophenyl)piperidine hydrochloride | 180.2 |
| 4-(3-methoxyphenyl)piperidine hydrochloride | 192 |
| 4-(3-trifluoromethyl)piperidine hydrochloride | 230 |
| 4-(3,5-difluorophenyl)piperidine hydrochloride | 198 |
| 4-(2-Methoxy-4-trifluoromethylphenyl)piperidine | 260 |
| hydrochloride | |
| 4-(2-Fluoro-4-trifluoromethylphenyl)piperidine hydrochloride | 248 |
| 4-(2-trifluoromethylphenyl)piperidine hydrochloride | 230 |
| 4-p-tolylpiperidine | 176 |
| 4-(2-Fluoro-5-trifluoromethylphenyl)piperidine hydrochloride | 248 |

### Method D

### Scheme VI

### Suzuki Coupling/Pyridine Hydrogenation

### Preparation 18

### 4-(2-Methoxyphenyl)piperidine hydrochloride salt

4-Pyridyl boronic acid (2.0 g, 16.3 mmol), 2-bromoanisole (2.0 g, 16.3 mmol) and tetrakis (triphenylphosphine) palladium (0) (2.0 g, 16.3 mmol) were combined in 100 mL of DME and 33 mL of H₂O under N₂ at room temperature. The reaction mixture was then heated to reflux at 85° C. for 17 hours. After cooling to room temperature, the mixture was partitioned between 300 mL brine and 300 mL ethyl acetate. The organic layer was separated and dried over anhydrous Na₂SO₄, filtered and the solvent was evaporated under vacuum. The residue was purified by flash column with 1:1 EtOAc:Hexane to give 865 mg of 4-(2-methoxyphenyl)pyridine as a colorless oil which crystallized under high vacuum. 400 MHz ¹H NMR (CDCl₃) δ (ppm) 8.6 (m, 2H) 7.5 (m, 2H), 7.3-7.4 (m, 2H), 7.0-7.1 (m, 2H), 3.8 (s, 3H); MS (M+1) 186.1. The product was converted to the HCl salt by dissolving the residue in EtOAc and adding 10 mL of 1 M HCl in diethyl ether. The solvent was removed in vacuo to 1.0 g of an off-white solid after drying under high vacuum.

4-(2-Methoxyphenyl)pyridine hydrochloride salt (1.0 g) was dissolved in methanol (23 mL) and platinum (IV) oxide (499 mg) was added. The mixture was then shaked on a Parr shaker under hydrogen (40 psi) for 90 minutes. Additional 500 mg of platinum (IV) oxide was added and the mixture was again placed on the Parr shaker for additional 2 hours. The reaction mixture was then filtered through a pad of celite and the cake was rinsed several times with CH₃OH. The filtrate was evaporated in vacuo to give 1.0 g of 4-(2-methoxyphenyl)piperidine HCl salt as a white solid. 400 MHz ¹H NMR (CDCl₃) δ 9.5-9.7 (broad d, 2H), 7.2 (m, 2H), 6.9 (m, 1H), 6.8-6.9 (d, 1H), 3.6 (s, 3H), 3.6 (d, 2H), 3.1-3.2 (m, 1H), 3.0 (q, 2H), 2.1-2.2 (m, 2H), 2.0 (d, 2H) MS (m/z) 192.0.

### Method E

### Scheme VI

### Suzuki Coupling/Pyridine Hydrogenation

### Preparation 19

### Cis-4-(2-Methoxy-4-(trifluoromethyl)phenyl)-3-methylpiperidine hydrochloride

To a stirred solution of 1-methoxy-3-(trifluoromethyl)benzene (9.8 mL, 68 mmol) in 50 mL THF under N₂ at 0° C. as added n-BuLi (1.6 M in hexanes, 45 mL, 68 mmol) dropwise. The reaction mixture was stirred at 0° C. for 2 h, then triisopropylborate (11.6 mL, 68 mmol) was added. The reaction mixture was slowly warmed up to room temperature and stirred overnight. A solution of 10% HCl in water was added and the mixture was stirred for 1 h. The mixture was extracted with CH₂Cl₂ (3×). The organic layers were combined, washed with brine and dried over Na₂SO₄ to give 8.14 g of 2-methoxy-4-(trifluoromethyl)phenylboronic acid as a viscous oil. The crude was directly used in the next step without further purification.

2-Methoxy-4-(trifluoromethyl)phenylboronic acid (8.14 g, 37 mmol), 4-bromo-3-methylpyridine HCl salt (1.3 g, 5.81 mmol), NaHCO₃ (6.0 g, 70 mmol) and tetrakis (triphenylphosphine) palladium (0) (671 mg, 0.58 mmol) were combined in 9 mL of DME and 9 mL of H₂O under N₂ at room temperature. The mixture was stirred for 10 min and then heated to reflux overnight. After cooling to room temperature, the mixture was partitioned between brine and ethyl acetate. The organic layer was separated and dried over anhydrous Na₂SO₄, filtered and the solvent was evaporated under vacuum. The residue was purified by flash column with 10% EtOAc in hexane to give 1.89 g of 4-(2-methoxy-4-(trifluoromethyl)phenyl)-3-methylpyridine. 400 MHz ¹H NMR (CDCl₃) δ (ppm) 8.5 (s, 1H), 8.45 (d, 1H), 7.30 (d, 1H), 7.22 (t, 1H), 7.17 (s, 1H), 7.06 (d, 1H), 3.8 (s, 3H), 2.1 (s, 3H), MS (M+1) 268. The product was converted to the HCl salt by dissolving the residue in CH₂Cl₂ and adding 2 mL of 4N HCl in dioxane. The solvent was removed in vacuo and the residue was triturated with diethyl ether to give 2.0 g of a pure white solid after filtration.

4-(2-Methoxy-4-(trifluoromethyl)phenyl)-3-methylpyridine hydrochloride salt (692 mg) was dissolved in ethanol (40 mL) and platinum (IV) oxide (70 mg) was added. The mixture was then shaked on a Parr shaker under hydrogen (40 psi) at 70° C. for 48 h. The reaction mixture was then filtered through a pad of celite and the cake was rinsed several times with ethanol. The filtrate was evaporated in vacuo and co-evaporated with diethyl ether (2×) to give 690 mg of Cis-4-(2-Methoxy-4-(trifluoromethyl)phenyl)-3-methylpiperidine HCl salt as a white solid. 400 MHz ¹H NMR (CD₃OD) δ (ppm) 7.30 (m, 2H), 7.20 (s, 1H), 3.9 (s, 3H), 3.40-3.60 (m, 2H), 3.20-3.29 (m, 2H), 3.1-3.18 (m, 1H), 2.55 (m, 1H), 2.40 (m, 1H), 1.76 (m, 1H), 0.80 (d, 3H); MS (m/z) 274, 315 (+CH₃CN). The enantiomers, 4-(R)-(2-Methoxy-4-(trifluoromethyl)phenyl)-3-(R)-methylpiperidine and 4-(S)-(2-Methoxy-4-(trifluoromethyl)phenyl)-3-(S)-methylpiperidine, were obtained through chiral separation.

The following cis-3,4-disubstituted piperidines were prepared as above starting with the reaction of substituted bromopyridine and the appropriate arylboronic acid:

TABLE 4

| NAME | m/z |
|---|---|
| Cis-4-(4-Chloro-2-fluorophenyl)-3-methylpiperidine hydrochloride | 228.1 |
| Cis-4-(3-Chloro-4-fluorophenyl)-3-methylpiperidine hydrochloride | 228.1 |
| Cis-4-(4-trifluormethylphenyl)-3-methylpiperidine hydrochloride | 244.2 |
| Cis-4-(4-trifluoromethylphenyl)-2-methylpiperidine hydrochloride | 244.2 |
| Cis-4-(4-ethoxyphenyl)-3-methylpiperidine hydrochloride | 220.2 |
| Cis-4-(3,4-difluorophenyl)-3-methylpiperidine hydrochloride | 212.1 |
| Cis-4-(2-Chloro-4-fluorophenyl)-3-methylpiperidine hydrochloride | 228.1 |
| Cis-4-(2-Fluorophenyl)-3-methylpiperidine hydrochloride | 194.2 |
| Cis-4-(4-Fluoro-2-methoxyphenyl)-3-methylpiperidine | 224.2 |
| hydrochloride | |
| Cis-4-(2,4-difluorophenyl)-3-methylpiperidine hydrochloride | 212.1 |
| Cis-4-(2-Fluoro-4-trifluoromethylphenyl)-3-methylpiperidine | 262.1 |
| hydrochloride | |
| Cis-4-(2-Fluoro-4-trifluoromethoxy phenyl)-3-methylpiperidine | 278.2 |
| hydrochloride | |
| Cis-4-(2-methoxyphenyl)-3-methylpiperidine hydrochloride | 206.2 |
| Cis-4-(3-fluoro-4-(trifluoromethyl)phenyl)-3-methylpiperidine | 262.2 |
| hydrochloride | |
| Cis-4-(2,4-bis(trifluoromethyl)phenyl)-3-methylpiperidine | 312.1 |
| hydrochloride | |
| Cis-4-(2-chloro-4-(trifluoromethyl)phenyl)-3-methylpiperidine | 278.2 |
| hydrochloride | |
| Cis-4-(2,4-dichlorophenyl)-3-methylpiperidine hydrochloride | 244.2 |
| Cis-4-(2-methyl-4-chloro)phenyl)-3-methylpiperidine | 224.2 |
| hydrochloride | |

### Method F

### Scheme XII

### Fluorination of Piperidine

### Preparation 19

### 4-(4-fluorophenyl)-4-fluoro-1-piperidine hydrochloride

A solution of [bis(2-methoxyethyl)amino]sulfur trifluoride (BAST) (0.475 ml, 2.6 mmol) in 20 ml of methylene chloride was cooled to −78° C. and a solution of tert-butyl 4-(4-fluorophenyl)-4-hydroxy-1-piperidinecarboxylate (760 mg, 2.6 mmol) (J. Med. Chem. 1992, 35 (22), 4020-26 or Bioorg. Med. Chem. Lett. 2003, 13 (22) 3951-4) in 10 ml of methylene chloride was added dropwise over 5 min. After stirring for 1 h, the mixture was warmed to room temperature, poured into saturated aqueous bicarbonate, and extracted 3 times with methylene chloride. The combinded organics were washed with brine, dried over sodium sulfate and concentrated under reduce pressure to provide 700 mg of tert-butyl 4-(4-fluorophenyl)-4-fluoro-1-piperidinecarboxylate as a yellow oil. MS m/z 298.2

A solution of tert-butyl 4-(4-fluorophenyl)-4-fluoro-1-piperidinecarboxylate (0.7 g) in 4M HCl/dioxane (15 ml) was stirred at room temperature. After 2 h the mixture was concentrated under reduced pressure to give 0.55 g of 4-(4-fluorophenyl)-4-fluoro-1-piperidine hydrochloride as an off-white solid. MS m/z 198.2.

The following 4-fluoro-4-aryl piperidines were prepared as above starting with the reaction of BAST and the appropriate tert-butyl 4-aryl-4-hydroxy-1-piperidinecarboxylate:

TABLE 5

| NAME | m/z |
|---|---|
| 4-fluoro-4-(4-chlorophenyl)-1-piperidine hydrochloride | 214.2 |
| 4-fluoro-4-(4-trifluoromethylphenyl)-1-piperidine hydrochloride | 248.2 |
| 4-fluoro-4-(2-methylphenyl)-1-piperidine hydrochloride | |

### Method G

### Scheme XIII

### Preparation 20

### 3-Hydroxy-4-(4-trifluoromethyl)phenyl)piperidine hydrochloride

To a stirred solution of borane-methylsulfide complex (0.1 mL) in THF (5 mL) under N₂ at 0° C. was added tert-butyl 4-(4-(trifluoromethyl)phenyl)-5,6-dihydropyridine-1(2H)-carboxylate (prepared according to preparation 13 using 4-trifluoromethylphenylboronic acid) (300 mg, 0.92 mmol) in THF (2 mL) dropwise. After the addition was complete, the reaction mixture was stirred at room temperature overnight, then cooled to 0° C. and sodium hydroxide (1N in water, 2 mL) was added dropwise, followed by hydrogen peroxide (2 mL). The resulting mixture was heated to 60° C. for 45 min, then cooled to room temperature and diluted with 20 mL CH₂Cl₂. The mixture was washed with water, brine and dried over NaₛSO_{d}. The solvent was removed in vacuo to give 278 mg of tert-butyl 3-hydroxy-4-(4-(trifluoromethyl)phenyl)piperidine-1-carboxylate as a mixture of diastereomers.

3-Hydroxy-4-(4-(trifluoromethyl)phenyl)piperidine-1-carboxylate (230 mg) was dissolved in CH₂Cl₂ (1.5 mL) and 0.2 mL of 4N HCl in dioxane was added. The mixture was stirred at room temperature overnight and the solvent was removed under reduced pressure to give 198 mg of 3-hydroxy-4-(4-trifluoromethyl)phenyl)piperidine hydrochloride. MS m/z 246.2.

### Preparation 21

### 3-Fluoro-4-(4-(trifluoromethyl)phenyl)piperidine hydrochloride

A solution of [bis(2-methoxyethyl)amino]sulfur trifluoride (BAST) (77 uL, 0.76 mmol) in 1.5 ml of methylene chloride was cooled to −78° C. and a solution of tert-butyl 3-hydroxy-4-(4-(trifluoromethyl)phenyl)piperidine-1-carboxylate (250 mg, 0.72 mmol) in 1 mL of methylene chloride was added dropwise over 5 min. After stirring for 1 h, the mixture was warmed to room temperature, poured into saturated aqueous bicarbonate, and extracted 3 times with methylene chloride. The combined organics were washed with brine, dried over sodium sulfate and concentrated under reduce pressure to provide 259 mg of tert-butyl 3-fluoro-4-(4-(trifluoromethyl)phenyl)piperidine-1-carboxylate as a yellow oil. The residue vas dissolved in CH₂Cl₂ (1.5 mL) and 0.2 mL of 4N HCl in dioxane was added. The mixture was stirred at room temperature overnight and the solvent was removed under reduced pressure to give 214 mg of 3-fluoro-4-(4-trifluoromethyl)phenyl)piperidine hydrochloride MS m/z 248.2.

### Method H

### Cyclopropanation

### Preparation 22

### 6-(4-Fluorophenyl)-3-aza-bicyclo[4.1.0]heptane

To a stirred solution of tert-butyl 4-(4-fluorophenyl)-1-(1,2,3,6-tetrahydropyridine) carboxylate (50 mg, 0.18 mmol) in dichloroethane at 0° C. was added a 1M solution of diethylzinc in cyclohexane (0.9 ml, 0.9 mmol) followed by diiodomethane (481 mg, 1.8 mmol). After 20 min the reaction mixture was warmed to room temperature. After 24 h the mixture was poured into saturated ammonium chloride and extracted with dichloromethane. The combined organic layers were washed with brine, dried over magnesium sulfate, filtered and concentrated under reduced pressure to a yellow residue [MS m/z 292.4]. The residue was dissolved in a 4M solution of HCl in dioxane (5 ml). After stirring for 1 h at room temperature the mixture was concentrated under reduced pressure to give 40 mg of 6-(4-fluorophenyl)-3-aza-bicyclo[4.1.0]heptane. MS m/z 192.3.

### Method I

### Reductive Animation Pyridine Hydrogenation

### Preparation 23

### (2S,6R)-4-(4-piperidin-4-yl)benzyl)-2,6-dimethylmorpholine

A solution of 4-(pyridine-4-yl)benzaldehyde (200 mg, 1.09 mmol) and cis-2,3-dimethylmorpholine (126.4 mg, 1.09 mmol) in 10 ml of dichloromethane was stirred at room temperature. After 1 h triethylamine (5 drops) and sodium triacetoxyborohydride (462 mg, 2.18 mmol) were added. After stirring for 24 h dichloromethane was added and the mixture was washed with 1N sodium hydroxide. The aqueous layer was back extracted with dichloromethane (100 mL×2) and the combined organics were dried over magnesium sulfate, filtered and concentrated under reduced pressure to give 180 mg of (2S,6R)-4-(4-pyridin-4-yl)benzyl)-2,6-dimethylmorpholine as a transparent oil. MS m/z 283.3.

A mixture of (2S,6R)-4-(4-pyridin-4-yl)benzyl)-2,6-dimethylmorpholine (180 mg), platinum(IV) oxide (10 mg), and 4N HCl in dioxane (1 mL) in methanol (10 mL) was shaken under 40 psi of hydrogen in a Parr shaker. After 4 h the Parr apparatus was purged with nitrogen and the reaction mixture was filtered through celite and concentrated under reduced pressure to give 180 mg of (2S,6R)-4-(4-piperidin-4-yl)benzyl)-2,6-dimethylmorpholine as a yellowish oil. MS m/z 289.3.

The references for the amines that are known in the literature are listed in Table 6.

TABLE 6

| IUPAC Name | References |
|---|---|
| 4-(3-trifluoromethyl-phenyl)-piperidin-4-ol | Collect. Czech. Chem. Commun., 1973, 38, |
| | 3829-3901 |
| 3H-Spiro[2-benzofuran-1,4′-piperidine] | J. Med. Chem., 1976, 19, 1315-1324 |
| Spiro[indene-1,4′-piperidine] | J. Med. Chem. 1992, 35, 3919-3927 |
| 1-(4-phenyl-piperidin-4-yl)-ethanone | J. Med. Chem. 1970, 13, 644-648 |
| 4-(4-chloro-3-trifluoromethyl-phenyl)- | Collect. Czech. Chem. Commun. 1973, 38, |
| piperidin-4-ol | 3879-3901 |
| 1-(4-phenyl-piperidin-4-yl)-propan-1-one | J. Chem. Soc. 1959, 1143-1147 |
| 4-phenyl-piperidine | Helv. Chim. Acta 1973, 56, 2348-2377 |
| 4-(4-bromo-phenyl)-piperidin-4-ol | J. Med. Chem. 1999, 42, 4680-4694 |
| 4-(4-chloro-phenyl)-piperidin-4-ol | J. Med. Chem. 1970, 13, 644-648 |
| 4-phenyl-piperidine-4-carbonitrile | J. Med. Chem. 1970, 13, 644-648 |
| 4-(2-isopropoxy-phenyl)-piperidine | J. Med. Chem. 1998, 41, 1997-2009 |
| 3-(3,4-dimethyl-piperidin-4-yl)-benzoic | Bioorg. Med. Chem. Lett. 2003, 13, 4459-4462 |
| acid methyl ester | |
| 3,5-dimethyl-4-phenyl-piperidin-4-ol | J. Med. Chem. 1964, 7, 726-728 |
| 1-methylspiro[indoline-1,4′-piperidine] | J. Med. Chem. 1997, 3905-3914 |
| 3-phenyl-pyrrolidine | J. Med. Chem. 1971, 14, 737-742 |
| 3-(4-fluoro-phenyl)-pyrrolidine | Bioorg. Med. Chem. Lett. 1999, 9, 1379-1384 |
| 3-benzyl-pyrrolidine | Med. Chem. Res. 1997, 7, 76-86 |
| 4-(2-chloro-phenyl)-piperidine | 1960, U.S. Pat. No. 2891066 |
| 4-(2-trifluoromethyl-phenyl)-piperidine | J. Med. Chem. 2005, 1857-1872 |
| Spiro[1-benzofuran-3,4′-piperidine] | J. Med. Chem. 1995, 38, 2009-2017 |
| 2-piperidin-4-yl-phenol | Chem. Pharm. Bull. 2000, 48, 1978-1985 |
| 5-phenyl-azepan-2-one | Chem. Pharm. Bull. 1969, 17, 434-453 |
| 4-(3,4-dimethyl-phenyl)-piperidine | Bioorg. Med. Chem. Lett. 1998, 8, 1499-1502 |
| 4-m-tolyl-piperidine | Chem. Pharm. Bull. 1987, 35, 2825-2839 |
| 3-(benzo[1,3]dioxol-5-yloxymethyl)-4-(4- | J. Med. Chem. 1997, 40, 1049-1062 |
| fluoro-phenyl)-piperidine | |
| 2-piperidin-4-yl-benzonitrile | Bioorg. Med. Chem. Lett. 2000, 10, 1917-1920 |
| 4-(3-chloro-phenyl)-piperidine | Eur. J. Med. Chem. Chim. Ther. 1987, 22, 337-346 |
| 3-piperidin-4-yl-benzonitrile | Bioorg. Med. Chem. 2005, 13, 2859-2872 |
| 4-(4-chloro-phenyl)-piperidine | Arzneim. Forsch. 1967, 17, 1145-1149 |
| 4-(4-methoxy-phenyl)-piperidine | NL 6510107 (1966) |
| 4-(4-ethyl-phenyl)-piperidine | DE 2801195 (1978) |
| 4-o-tolyl-piperidine | Bioorg. Med. Chem. Lett. 1998, 8, 1851-1856 |
| 4-(3-fluoro-phenyl)-piperidine | Chem. Pharm. Bull. 1987, 35, 2825-2839 |
| 4-p-tolyl-piperidine | Collect. Czech. Chem. Commun. 1975, 40, |
| | 3904-3923 |
| (R)-3-benzyl-pyrrolidine | Tetrahedron Lett. 1994, 35, 973-976 |
| (S)-3-benzyl-pyrrolidine | Tetrahedron Lett. 1994, 35, 973-976 |
| (R)-3-phenyl-pyrrolidine | Acta Chem. Scand. 1990, 44, 42-49 |
| (S)-3-phenyl-pyrrolidine | Synthesis 1991, 1023-1026 |
| (S)-5-phenyl-azepan-2-one | J. Am. Chem. Soc. 1990, 112, 4879-4891 |

Preparation of Compounds of Formula (I)

For each method, a general procedure or a representative synthesis is described. Other examples prepared via similar method using the appropriate intermediates and reagents are listed in table 7 with method number indicated.

### Method J

0.25 M stock solutions of amines (II) and aldehydes (III) in DCE were prepared. When applicable, the aldehyde salt forms were neutralized by addition of 4 equivalents of DIPEA. A 0.25 M fine suspension of NaBH(OAc)₃ in anhydrous DMF/DCE mixture (20/80) was prepared. To each vial was added 0.2 mL of a solution of amine (II) followed by 0.2 mL of a solution of aldehyde (III) and 0.5 mL of the NaBH(OAc)₃ suspension to each vial. The vials were capped and shaken at room temperature for 16 h. Additional 0.5 mL of the NaBH(OAc)₃ suspension was added to each vial, the vials were vortexed, capped, and shaken at room temperature for 16 h. The solvent was removed under reduced pressure. 1 mL of DMSO and 0.1 mL of water were added to each vial. The samples were vortexed for 1 h, 0.05 mL of concentrated NH₄OH vas added to each vial. The samples were filtered and directly submitted to HPLC purification.

### Method K

### Example 1

### Corresponding to Entry 354 in Table 7

### 2-((4-(2-Methoxy-4-trifluoromethyl)phenyl)piperidin-1-yl)methyl)-1-methyl-1H-benzo[d]imidazole

To a stirred solution of 4-(2-methoxy-4-(trifluoromethyl)phenyl)piperidine hydrochloride salt (444 mg, 1.5 mmol) in CH₂Cl₂ under N₂ at room temperature vas added triethylamine (1.7 mL, 12.0 mmol), MgSO₄ (20 mg) and 1-methyl-1H-benzo[d]imidazole-2-carbaldehyde (240 mg, 1.5 mmol). The reaction mixture was stirred for 30 min, then NaBH(OAc)₃ (477 mg, 2.25 mmol) was added. The mixture was stirred at room temperature overnight and was then diluted with methylene chloride (50 mL) and washed with water, brine and dried with Na₂SO₄. The solvent was removed in vacuo and the residue was purified by flash column with 1-5% MeOH in CH₂Cl₂ to give 546 mg of 2-((4-(2-Methoxy-4-(trifluoromethyl)phenyl)piperidin-1-yl)methyl)-1-methyl-1H-benzo[d]imidazole as a white foam. The residue was dissolved in MeOH (3 mL) and a 4N HCl solution in dioxane (0.4 mL) a added and the mixture was stirred for 10 min. The solvent was removed in vacuo to give 596 mg of 2-((4-(2-Methoxy-4-trifluoromethyl)phenyl)piperidin-1-yl)methyl)-1-methyl-1H-benzo[d]imidazole hydrochloride salt as a white solid, 400 MHz ¹H NMR (CD₃OD) δ (ppm) 7.75 (dd, 2H), 7.49 (m, 2H) 7.40 (d, 1H), 7.26 (d, 1H), 7.21 (s, 1H), 4.73 (s, 2H), 4.03 (s, 3H), 3.91 (s, 3H), 3.76 (m, 2H), 3.20-3.30 (m, 3H), 2.05-2.20 (m, 4H); MS (m/z) 404.2.

### Method K1

### Reductive Amination+Chiral Separation

### Example 2

### Corresponding to Entry 462 in Table 7

### 2-{[Cis-4-(3,4-Difluoro-phenyl)-3-methyl-piperidin-1-yl]methyl}-1-methyl-1H-benzoimidazole

To a stirred solution of cis-4-(3,4-Difluoro-phenyl)-3-methyl-piperidine hydrochloride salt (2.76 g, 11.1 mmol) in CH₂Cl₂ (50 mL) under N₂ at room temperature was added triethylamine (11.5 mL), MgSO₄ (1.0 g) and 1-methyl-1H-benzo[d]imidazole-2-carbaldehyde (1.78 g, 11.1 mmol). The reaction mixture was stirred at rt for 30 min, then NaBH(OAc)₃ (3.54 g, 16.7 mmol) was added. The mixture was stirred at room temperature overnight and was then diluted with methylene chloride (100 mL) and washed with water, brine and dried with Na₂SO₄. The solvent was removed in vacuo and the residue was purified by flash column with EtOAc to give 2.93 g of 2-{[cis-4-(3,4-Difluoro-phenyl)-3-methyl-piperidin-1-yl]methyl}-1-methyl-1H-benzoimidazole as a racemic mixture. 400 MHz ¹H NMR (CD₃OD) δ (ppm) 7.80 (d, H), 7.50 (d, 1H), 7.28 (m, 2H), 7.10 (m, 1H), 7.08 (m, 1H), 6.96 (m, br, 1H), 3.92 (s, 3H), 3.80 (s, 2H), 3.00 (m, 1H), 2.85 (m, 1H), 2.78 (d, 1H), 2.48 (d, 1H), 2.20 (m, 1H), 2.10-2.17 (m, 2H), 1.60 (d, br, 1H), 0.76 (d, 3H); MS⁺ (m/z) 356.1.

Two enantiomers were separated using a Chiralpak AD-H column (2.1 CM×25 CM), with 80/20 CO₂/MeOH as Mobile phase at flow rate of 65 g/min to obtain two enantiomers.

### Example 2a

### Corresponding to Entry 472 in Table 7

### 2-{[Cis-4-(3,4-Difluoro-phenyl)-3-methyl-piperidin-1-yl]methyl}-1-methyl-1H-benzoimidazole, Enantiomer #1: Retention time: 6.121 min, 100% ee

### Example 2b

### Corresponding to Entry 473 in Table 7

### 2-{[Cis-4-(3,4-Difluoro-phenyl)-3-methyl-piperidin-1-yl]methyl}-1-methyl-1H-benzoimidazole, Enantiomer #2: Retention time: 9.82 min, 95% ee

### Method L

0.2 M Stock solutions of amine (II) and alkyl halide (IV) in DMF were prepared. Using a solid reagent dispenser, ˜25 mg of K₂CO₃ and ˜10 mg of NaI were placed into each reaction vial. 0.25 mL of a solution of amine (II) and 0.25 mL of a solution of alkyl halide (IV) were added to the reaction vials. The vials were capped and shaken at 80° C. for 15 h. The DMF was removed by evaporation under the reduced pressure and 1.1 mL of 5% MeOH solution in CH₂Cl₂ was added to each vial. The reactions were vortexed and allowed to stand for 30 min, the samples were transferred to a filter plate containing filter plugs of 0.25 mL each of silica gel and cellulose. The filter plugs were washed with 1 mL of the 5% MeOH solution in CH₂Cl₂. The samples were evaporated to dryness under the reduced pressure and the residue was submitted to HPLC purification.

### Method M

### Example 3

### Corresponding to Entry 325 in Table 7

### 4-Fluoro-2-((4-(2-Fluorophenyl)piperidin-1-yl)methyl)-1-methyl-1H-benzo[d]imidazole

To a stirred solution of 4-(2-fluorophenyl)piperidine (50 mg, 0.28 mmol) in DMF (1 mL) under N₂ at room temperature was added Na₂CO₃ (59 mg, 0.56 mmol). TBAI (103 mg, 0.28 mmol) and 2-(chloromethyl)-4-fluoro-1-methyl-1H-benzo[d]imidazole (55 mg, 0.28 mmol). The mixture was heated to 105° C. overnight. LC-MS indicated no starting material was left. The reaction mixture was cooled to room temperature and partitioned between 1N NaOH aqueous solution (10 mL) and EtOAc (10 mL). The organic layer was washed with water, brine and dried with Na₂SO₄. The solvent was removed in vacuo and the residue was purified by flash column with 100% EtOAc to give 25 mg of 4-fluoro-2-((4-2-fluorophenyl)piperidin-1-yl)methyl)-1-methyl-1H-benzo[d]imidazole as a yellow paste. 400 MHz ¹H NMR (CD₃OD) δ (ppm) 7.31 (d, 1H), 7.25 (m, 2H), 7.14 (m, 1H), 7.08 (m, 1H), 6.98 (m, 2H), 3.94 (s, 3H), 3.85 (s, 2H), 3.00 (m, 2H), 2.86 (m, 1H), 2.28 (m, 2H), 1.78 (m, 4H); MS (m/z) 342.2.

### Method M1

### Example 4

### Corresponding to Entry 272 in Table 7

### 2-((4-(2-Fluorophenyl)piperidin-1-yl)methyl)-1-methyl-1H-benzo[d]imidazole-5-carbonitrile

To a stirred solution of 4-(2-fluorophenyl)piperidine (600 mg) in DMF (5 mL) under N₂ at room temperature was added Na₂CO₃ (711 mg), TBAI (1.76 g) and 2-(chloromethyl)-1-methyl-1H-benzo[d]imidazole-5-carbonitrile (687 mg). The mixture was heated in a microwave reactor to 170° C. for 1 h. LC-MS indicated no starting material was left. The reaction mixture was partitioned between 1N NaOH aqueous solution and EtOAc. The organic layer was washed with water, brine and dried with Na₂SO₄. The solvent was removed in vacuo and the residue was purified by flash column with 95-100% EtOAc in hexane to give 418 mg of 2-((4-(2-fluorophenyl)piperidin-1-yl)methyl)-1-methyl-1H-benzo[d]imidazole-5-carbonitrile. 400 MHz ¹H NMR (CD₃OD) δ (ppm) 8.0 (s, 1H), 7.68 (d, 1H), 7.60 (d, 1H), 7.27 (m, 1H), 7.17 (m, 1H), 7.08 (m, 1H), 7.00 (m, 1H), 3.98 (s, 3H), 3.89 (s, 2H), 3.00 (m, 2H), 2.87 (m, 1H), 2.27 (m, 2H), 1.79 (m. 4H); MS (m/z) 349.1.

### Method N

### Example 5

### Corresponding to Entry 291 in Table 7

### 5-(2-Methoxy-phenyl)-1-(1-methyl-1H-benzoimidazol-2-ylmethyl)-azepan-2-one

To a stirred solution of 5-(2-methoxyphenyl)azepan-2-one (200 mg, 0.91 mmol) in DMF (5 mL) under N₂ at room temperature was added NaH (60% in mineral oil, 36 mg, 1.4 mmol), TBAI (100 mg, 0.27 mmol) and 2-(chloromethyl)-1-methyl-1H-benzo[d]imidazole (213 mg, 1.2 mmol). The mixture was stirred at room temperature overnight LC-MS indicated no starting material was left. The reaction mixture was partitioned between 1N NaOH aqueous solution and EtOAc. The organic layer was washed with water, brine and dried with Na₂SO₄. The solvent was removed in vacuo and the residue was purified by flash column with 5-10% MeOH in CH₂Cl₂ and to give 166 mg of 5-(2-Methoxy-phenyl)-1-(1-methyl-1H-benzoimidazol-2-ylmethyl)-azepan-2-one MS (m/z) 364.1.

### Method O

### Scheme VIII

### Example 6

### Corresponding to Entry 275 in Table 7

### 2-((4-(2-Fluorophenyl)piperidin-1-yl)methyl)-3-methyl-3H-benzo[d]imidazole-5-carbonitrile

A mixture of 6-bromo-2-((4-(2-fluorophenyl)piperidin-1-ylmethyl)-1-methyl-1H-benzo[d]imidazole (50 mg), zinc cyanide (21.3 mg), Pd(dppf)Cl₂ (15 mg) and 1 ml of dimethyl formamide was heated to 120° C. for 10 minutes under microwave (CEM). The mixture filtered over diatomaceous earth and the solvent was removed in vacuo. The residue was purified using silica gel chromatography (0% to 50% Hexane/EtOAc) to yield 2-((4-(2-fluorophenyl)piperidin-1-yl)methyl)-3-methyl-3H-benzo[d]imidazole-5-carbonitrile (15.9 mg). MS (M+1) 349.1.

### Method P

### Scheme VIII

### Example 7

### Corresponding to Enter 276 in Table 7

### 2-((4-(2-Fluorophenyl)piperidin-1-yl)methyl)-1-methyl-6-phenyl-1H-benzo[d]imidazole

A mixture of 6-bromo-2-((4-(2-fluorophenyl)piperidin-1-yl)methyl)-1-methyl-1H-benzo[d]imidazole (50 mg), phenylboronic acid (22 mg), Tetrakis(triphenylphosphine) palladium (0), (7 mg), 0.1 ml saturated NaHCO₃ solution, 0.5 ml of toluene and 0.5 ml of ethanol was heated to 120° C. for 10 minutes under microwave (CEM). The mixture filtered over diatomaceous earth and the solvent was removed in vacuo. The residue was purified using silica gel chromatography (0% to 60% Hexane/EtOAc) to yield 2-((4-(2-fluorophenyl)piperidin-1-yl)methyl)-1-methyl-6-phenyl-1H-benzo[d]imidazole (10.2 mg). MS (m/z) 400.2.

### Method Q

### Scheme VIII

### Example 8

### Corresponding to Entry 278 in Table 7

### 2-((4-(2-fluorophenyl)piperidin-1-yl)methyl)-6-methoxy-1-methyl-1H-benzo[d]imidazole

A mixture of 6-bromo-2-((4-(2-fluorophenyl)piperidin-1-yl)methyl)-1-methyl-1H-benzo[d]imidazole (50 mg), copper(I) iodide (31 mg), CsCO₃ (43 mg), 1,10-phenanthroline (4.3 mg) and 1 ml of methonal was heated to 120° C. for 1 ours, followed by 130° C. for 30 minutes under microwave (CEM). The mixture filtered over diatomaceous earth and the solvent was removed in vacuo. The residue was purified using silica gel chromatography (0% to 60% Hexane/EtOAc) to yield 2-((4-(2-fluorophenyl)piperidin-1-yl)methyl)-6-methoxy-1-methyl-1H-benzo[d]imidazole (12.5 mg, MS (m/z) 354.1

### Method R

### Scheme VIII

### Example 9

### Corresponding to Entry 279 in Table 7

### 2-((4-2-fluorophenyl)piperidin-1-yl)methyl)-1,6-dimethyl-1H-benzo[d]imidazole

A mixture of 6-bromo-2-((4-(2-fluorophenyl)piperidin-1-yl)methyl)-1-methyl-1H-benzo[d]imidazole (50 mg), dimethyl zinc (0.12 ml). Pd(dppf)Cl₂ (27 mg) and 1 ml of dioxane was heated to 120° C. for 10 minutes under microwave (CEM). The mixture filtered over diatomaceous earth and the solvent was removed in vacuo. The residue was purified using acidic resin to yield 2-((4-(2-fluorophenyl)piperidin-1-yl)methyl)-1,6-dimethyl-1H-benzo[d]imidazole (43.1 mg). MS (m/z) 338.1.

### Method R1

### Scheme VIII

### Example 10

### Corresponding to Entry 264 in Table 7

### 2-[4-(2-Methoxy-phenyl)-piperidin-1-ylmethyl]-1-methyl-1H-benzoimidazol-4-ylamine

A mixture of 5-bromo-2-((4-(2-methoxyphenyl)piperidin-1-yl)methyl)-1-methyl-1H-benzo[d]imidazole (100 mg, 0.26 mmol), diphenylmethanimine (48 uL, 0.29 mmol), NaOt-Bu (43 mg, 0.34 mmol), BINAP (120 mg, 0.18 mmol) and Pd₂(dba)₃ (55 mg, 0.06 mmol) in toluene was heated to 110° C. for 10 minutes under microwave (CEM) for 10 min. The mixture filtered over diatomaceous earth and the solvent was removed in vacuo. The residue was taken in a 1:1 mixture of 1N HCl aqueous solution: THF (4 mL) and the mixture was refluxed for 1 h. The mixture was concentrated and partitioned between 1N HCl aqueous solution and EtOAc. The aqueous layer was basified with 1N NaOH aqueous solution and extracted with methylene chloride (3×10 mL). The combined organic layers were washed with brine and dried with Na₂SO₄. The solvent was removed in vacuo and the residue was purified by flash column with gradient 5-95% EtOAc in hexane to yield 2-[4-(2-Methoxy-phenyl)-piperidin-1-ylmethyl]-1-methyl-1H-benzoimidazol-4-ylamine (51 mg). MS (m/z) 351.

### Method S

### Scheme VIII

### Example 11

### Corresponding to Entry 296 in Table 7

### Methyl 2-((4-(2-methoxyphenyl)piperidin-1-yl)methyl)-1-methyl-1H-benzo[d]imidazole-5-carboxylate

A mixture of 5-bromo-2-((4-(2-methoxyphenyl)piperidin-1-yl)methyl)-1-methyl-1H-benzo[d]imidazole (150 mg), triethylamine (63 uL), Pd(dppf)Cl₂.CH₂Cl₂ (53 mg) and 40 mL of methanol was heated to 70° C. under CO (20-30 psi) overnight. The mixture vas filtered through a pad of celite and the cake WAS rinsed with methanol. The combined filtrate was concentrated in vacuo and the residue was purified by flash column with 80-100% EtOAc in hexane to yield methyl 2-((4-(2-methoxyphenyl)piperidin-1-yl)methyl)-1-methyl-1H-benzo[d]imidazole-5-carboxylate (74 mg). MS (m/z) 394.2.

### Method S1

### Scheme VIII

### Example 12

### Corresponding to Entry 288 in Table 7

### 2-[4-(2-Methoxy-phenyl)-piperidin-1-ylmethyl]-1-methyl-1H-benzoimidazole-5-carboxylic acid

To a stirred solution of methyl 2-((4-(2-fluorophenyl)piperidin-1-yl)methyl)-1-methyl-1H-benzo[d]imidazole-5-carboxylate (74 mg, 0.28 mmol) in MeOH (1 mL) was added 1N NaOH aqueous solution (1 mL) and the reaction mixture was heated to 50° C. and stirred overnight. The mixture was acidified with 1N HCl to PH 6 and partitioned between water and methylene chloride. The organic layer was washed with water, brine and dried with Na₂SO₄. The solvent was removed in vacuo to yield 2-[4-(2-methoxy-phenyl)-piperidin-1-ylmethyl]-1-methyl-1H-benzoimidazole-5-carboxylic acid (68 mg). MS (m/z) 378, 380.

### Method T

### Example 13

### Corresponding to Entry 298 in Table 7

### 2-(2-((4-(2-fluorophenyl)piperidin-1-yl)methyl)-1-methyl-1H-benzo[d]imidazol-5-yl)propan-2-ol

To a stirred solution of methyl 2-((4-(2-fluorophenyl)piperidin-1-yl)methyl)-1-methyl-1H-benzo[d]imidazole-5-carboxylate (78 mg, 0.2 mmol) in THF (1 mL) under N₂ at 0° C. was added a solution of MeMgBr in THF/toluene (0.26 mL, 0.4 mmol) and the reaction mixture was slowly warmed to room temperature. There was still starting material left. The mixture was cooled to −60° C. and MeLi (1.6 M in Et₂O, 0.26 mL) was added and the mixture was stirred for 10 min and then partitioned between NH₄Cl aq and methylene chloride. The organic layer was washed with water, brine and dried with Na₂SO₄. The solvent was removed in vacuo and the residue was purified by flash column with 85-100% EtOAc in hexane to yield 2-(2-((4-(2-fluorophenyl)piperidin-1-yl)methyl)-1-methyl-1H-benzo[d]imidazol-5-yl)propan-2-ol (17 mg) MS (m/z) 382.

### Method U

### Example 14

### Corresponding to Entry 297 in Table 7

### (2-((4-(2-Fluorophenyl)piperidin-1-yl)methyl)-1-methyl-1H-benzo[d]imidazol-5-yl)methanol

To a stirred solution of methyl 2-((4-(2-fluorophenyl)piperidin-1-yl)methyl)-1-methyl-1H-benzo[d]imidazole-5-carboxylate (98 mg, 0.26 mmol) in THF (1 mL) under N₂ at 0° C. was added LiAlH₄ (1M solution in THF, 0.26 mL, 0.26 mmol) and the reaction mixture was slowly warmed to room temperature. The reaction was carefully quenched with NH₄Cl aq and the mixture was extracted with methylene chloride. The organic layer was washed with water, brine and dried with Na₂SO₄. The solvent was removed in vacuo and the residue was purified by flash column with 95-100% EtOAc in hexane to yield (2-((4-(2-Fluorophenyl)piperidin-1-yl)methyl)-1-methyl-1H-benzo[d]imidazol-5-yl)methanol (102 mg). MS (m/z) 354.

### Method V

### Example 15

### Corresponding to Entry 363 in Table 7

### 2-((4-(2-Fluorophenyl)piperidin-1-yl)methyl)-1-methyl-1H-benzo[d]imidazol-5-carboxamide

To a stirred solution of 2-((4-(2-fluorophenyl)piperidin-1-yl)methyl)-1-methyl-1H-benzo[d]imidazole-5-5-carbonitrile (50 mg, 0.12 mmol) in t-BuOH (1 mL) was added KOH (33 mg, 0.60 mmol) and the reaction mixture was heated to reflux overnight. The reaction mixture was cooled to room temperature and the mixture was partitioned between H₂O and methylene chloride. The organic layer was washed with brine and dried with Na₂SO₄. The solvent was removed in vacuo and the resultant yellow solid was converted to the HCl salt with 1.2 eq of 4N HCl in dioxane. The salt was then triturated with ethyl ether to yield 2-((4-(2-fluorophenyl)piperidin-1-yl)methyl)-1-methyl-1H-benzo[d]imidazole-5-carboxamide (39 mg). MS (m/z) 367.

### Method W

### Scheme VII

Stock solutions of amines (XXIX) (0.15 M in THF), PPh₃ (0.5 M in THF), and di-t-butylazodicarboxylate (0.3 M in THF) were prepared. The vials containing alcohols of formula (XXX) was added 1.2 mL of THF and the mixtures was sonicated. To each vial was added 0.667 mL of the solution of amine (XXIX), 0.50 mL of the PPh₃ solution, and 0.667 mL of the di-t-butylazodicarboxylate solution. The vials were capped and shaken at room temperature for 16 h. The solvent was evaporated under the reduced pressure and the residues were dissolved in 1 mL of MeOH. The obtained solutions were loaded onto Waters Oasis MCX cartridges (6 cc/500 mg) previously conditioned with 2 mL of MeOH. The vials were rinsed with 1 mL of methanol and the obtained solutions were loaded on the cartridges as well. The cartridges were eluted using 4.5 mL of 1 M NH₂ in MeOH into collection vials and the solvents were removed under nitrogen at 35° C.

Stock solutions of aldehyde (III) (0.25 M in DCE), and NaBH(OAc)₃, (0.25 M in CHCl₃) were prepared. The residue in each reaction vial was dissolved in 0.6 mL of DCE. To each vial was added 0.4 mL of the solution of aldehyde (III) and 1.2 mL of NaBH(OAc)₃ solution. The vials were capped and shaken at room temperature for 16 h. 2 ml of 10% aqueous NH₄OH was added to each vial and the mixtures were vortexed thoroughly. The mixtures were loaded onto Varian ChemElut cartridges and eluted with DCE (2×3 mL) into collection vials. The solvents were removed under nitrogen at 35° C. The residues were dissolved in 1 mL of DMSO, filtered and submitted to HPLC purification.

### Method X

### Scheme XI

2.0 M stock solutions of amine (XLVII) and 0.25 M solution of aldehyde (III) in DCE were prepared. A 0.25 M sodium triacetoxyborohydride suspension in chloroform was prepared and sonicated for 5 min. 1 mL of the solution of amine (XLVII) was dispensed into 2-dram vials. To each vial vas added 0.4 mL of the solution of aldehyde (III), 0.4 mL of the solution of amine (XLVII), and 1.28 mL of the sodium triacetoxyborohydride solution. The vials were capped and shaken at room temperature for 16 h. 2 mL of 10% aqueous NH₄OH was added to each vial. The vials were initially slowly then more vigorously vortexed and then centrifuged. 2 mL of the organic (lower) layer was transferred into collection vials. To each reaction vial was added 1 mL of DCE and the vials were vortexed well and centrifuged until the layers are well separated. The remaining organic layer was transferred to the collection vials. The solvent was removed by evaporation.

To each vial was added 0.8 mL of MeOH and 0.2 mL of 4 M HCl in dioxane. The vials were capped, vortexed, and shaken at room temperature for 24 h. The samples were evaporated to dryness. MCX cartridges were conditioned with 2 mL of MeOH. Samples were dissolved in 1 mL of MeOH, and then loaded onto the MCX cartridges. Cartridges were washed with 4.5 mL of MeOH. Free amine intermediates were then eluted with 4.5 mL of 1 M NH₃ in MeOH into a new set of collection vials. The solvent was removed by evaporation. To each vial was added 0.4 mL of DCE and the vials were shaken at room temperature until dissolution occurred. A 0.25 M solution of various aldehydes in DCE and 0.25 M suspension of NaBH(OAc)₃ in chloroform were prepared. To each via was added 0.4 mL of aldehyde solution and 1.28 mL of NaBH(OAc)₃ suspension. The vials were vortexed well, capped, and shaken at room temperature for 16 h. To each reaction vial was added 2 mL of 10% aqueous NH₄OH and the vials were vortexed well and centrifuged until the layers were well separated. 2 mL of the organic (lower) layer was transferred into collection vials. To each reaction vial was added 1 mL of DCE and the vials were vortexed well and centrifuged until the layers are well separated. The remaining organic layer was transferred into the collection vials. The samples were evaporated, dissolved in 1 mL of DMSO, filtered and submitted to HPLC purification.

### Method Y

### Example 16

### Corresponding to Entry 104 in Table 7

### 5-Bromo-1-methyl-1′-[(1-methyl-1H-benzimidazole-2-yl)methyl]-1,2-dihydrospiro[indole-3,4′-piperidine]

tert-Butyl 5-Bromo-1,2-dihydrospiro[indole-3,4′-piperidine]-1′-carboxylate (36.7 mg, 0.1 mmol), paraformaldehyde (15 mg), and sodium triacetoxyborohydride (63.6 mg, 0.3 mmol) in DCE (1 mL) were shaken at room temperature overnight. The reaction was washed with 10% NH₄OH (1 mL), and the biphasic mixture was loaded onto a 1 mL aq. capacity Varian ChemElut cartridge. Cartridge was eluted with DCE (2×3 mL), and the solvent removed under a stream of N₂ to give 31.8 mg residue which was used directly in the next step without further purification. LC/MS (acidic gradient) shows 81% (UV 215 nm), M+=381.0 (3.62 min).

Crude intermediate was dissolved in MeOH (800 uL), 4M HCl/dioxane (200 uL) was added and the reaction shaken at room temperature for 2.5 h. Solvents removed under a stream of N₂. Residue dissolved in MeOH (1000 uL), loaded onto a Waters Oasis MCX cartridge (400 mg sorbent), which had been preconditioned with 1000 uL MeOH. The cartridge was washed with MeOH (5 mL), and the deprotected intermediate was eluted with 1M NH₃ in MeOH (5 mL), and dried under a stream of N₂. The free amine was dissolved in DCE (1 mL); 1-methyl-1H-benzo[d]imidazole-2-carbaldehyde (13.4 mg, 0.08 mmol) and sodium triacetoxyborohydride 53.0 mg, 0.25 mmol) were added, and the reaction was shaken at room temperature overnight. The reaction was washed with 10% NH₄OH (1 mL), and the biphasic mixture was loaded onto a 1 mL aq. capacity Varian ChemElut cartridge. Cartridge eluted with DCE (2×3 mL), and the solvent removed under a stream of N₂. Crude product was purified using preparative HPLC (TFA conditions) to give 30.7 mg white solid as the bis-TFA salt. LC/MS (acidic gradient) shows 92% (UV 215 nm)/100% (ELSD), M+=426.9 (2.05 min).

### Method Z

### Scheme X

A 2.0 M solution of triethylamine solution in DCE was prepared 2.0 M stock solutions of amine (XLVII) in this mixture were prepared and sonicated for 10 min. A 0.25 M tetramethylammonium triacetoxyborohydride solution in DCE was prepared and sonicated for 10 min. 0.25 M solutions of aldehyde (III) in DCE were prepared. 1 mL of the solution of amine (XLVII) was dispensed into 2-dram vials. To each vial was added 0.8 mL of the solution of aldehyde (III), and 2.4 mL of the tetramethylammonium triacetoxyborohydride solution. The vials were capped and shaken at room temperature for 16 h. 1.5 mL of 15% NH₄OH aqueous solution was added to each vial. The vials were initially slowly then more vigorously vortexed, centrifuged, and allowed to stand for 2 h. The aqueous layer was discarded and the organic layers were evaporated 1 mL of DCM and 0.25 mL of TFA was added to each vial. The vials were capped and shaken at room temperature for 16 h. The solvent was evaporated and 1 mL of toluene was added to each vial. The samples were evaporated to dryness. 2 M solution of Et₃N and 0.25 M solutions of various acid chlorides in anhydrous DMA were prepared. 1 mL of the Et₃N solution was added to each vial and the vials were vortexed until the solutions became clear. 0.96 of the acid chloride solution was added to the corresponding vial and the vials were capped and shaken at room temperature for 16 h. The solvent was evaporated. To each vial was added 3 mL of DCE and 2 mL of water. The vials were vortexed and centrifuged. The organic layers were transferred into collections vial. The samples were evaporated, dissolved in 1 mL of DMSO, filtered and submitted to HPLC purification.

TABLE 7
Examples with Data
The following specific compounds were prepared following the similar procedures to
the preparations and examples described above, using appropriate intermediates and
reagents.

| | | EC50 | | Exact | | Retention | |
|---|---|---|---|---|---|---|---|
| Entry # | IUPAC name | (uM) | ParentMF | Mass | M/z | time (min) | Procedure |
| 1 | 1-(1-Methyl-1H- | >6.17 | C21H22F3N3O | 389.17 | 390.172 | 2.16 | J |
| | benzoimidazol-2- | | | | | | |
| | ylmethyl)-4-(3- | | | | | | |
| | trifluoromethyl-phenyl)- | | | | | | |
| | piperidin-4-ol | | | | | | |
| 2 | 1′-[(1-methyl-1H- | 1.81 | C21H23N3O | 333.18 | 335.051 | 1.38 | J |
| | benzimidazol-2- | | | | | | |
| | yl)methyl]-3H-spiro[2- | | | | | | |
| | benzofuran-1,4′- | | | | | | |
| | piperidine] | | | | | | |
| 3 | 2-[4-(4-Fluoro-phenyl)- | 0.0979 | C20H22FN3 | 323.18 | 324.1 | | J |
| | piperidin-1-ylmethyl]-1- | | | | | | |
| | methyl-1H- | | | | | | |
| | benzoimidazole | | | | | | |
| 4 | 1′-[(1-methyl-1H- | 0.439 | C22H23N3 | 329.19 | 331.15 | 1.24 | J |
| | benzimidazol-2- | | | | | | |
| | yl)methyl]spiro[indene- | | | | | | |
| | 1,4′-piperidine] | | | | | | |
| 5 | 1-butyryl-5-fluoro-1′-[(1- | 0.916 | C25H29FN4O | 420.23 | 420.2319 | 1.06 | Z |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 6 | 5-fluoro-1-(2-furoyl)-1′- | 2.62 | C26H25FN4O2 | 444.2 | 444.1956 | 1.04 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 7 | 1-(2,2- | 3.1 | C26H31FN4O | 434.25 | 434.2475 | 1.12 | Z |
| | dimethylpropanoyl)-5- | | | | | | |
| | fluoro-1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 8 | 1-(cyclopropylcarbonyl)- | 1.62 | C25H27FN4O | 418.22 | 418.2163 | 1.03 | Z |
| | 5-fluoro-1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 9 | 1-(cyclobutylcarbonyl)-5- | 2.17 | C26H29FN4O | 432.23 | 432.2319 | 1.08 | Z |
| | fluoro-1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 10 | methyl 4-{5-fluoro-1′-[(1- | 5.21 | C26H29FN4O3 | 464.22 | 464.2217 | 1.01 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 4-oxobutanoate | | | | | | |
| 11 | ethyl 4-{5-fluoro-1′-[(1- | 3.64 | C27H31FN4O3 | 478.24 | 478.2373 | 1.07 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 4-oxobutanoate | | | | | | |
| 12 | 5-fluoro-1′-[(1-methyl-1H- | 0.569 | C29H29FN4O2 | 484.23 | 484.2268 | 1.15 | Z |
| | benzimidazol-2- | | | | | | |
| | yl)methyl]-1- | | | | | | |
| | (phenoxyacetyl)-1,2- | | | | | | |
| | dihydrospiro[indole-3,4′- | | | | | | |
| | piperidine] | | | | | | |
| 13 | 4-({5-fluoro-1′-[(1-methyl- | 2.53 | C29H26FN5O | 479.21 | 479.2116 | 1.1 | Z |
| | 1H-benzimidazol-2- | | | | | | |
| | yl)methyl]spiro[indole- | | | | | | |
| | 3,4′-piperidin]-1(2H)- | | | | | | |
| | yl}carbonyl)benzonitrile | | | | | | |
| 14 | 3-({5-fluoro-1′-[(1-methyl- | 0.914 | C29H26FN5O | 479.21 | 479.2116 | 1.09 | Z |
| | 1H-benzimidazol-2- | | | | | | |
| | yl)methyl]spiro[indole- | | | | | | |
| | 3,4′-piperidin]-1(2H)- | | | | | | |
| | yl}carbonyl)benzonitrile | | | | | | |
| 15 | 1-[(3,5-dimethylisoxazol- | 5.21 | C27H28FN5O2 | 473.22 | 473.2221 | 1.03 | Z |
| | 4-yl)carbonyl]-5-fluoro-1′- | | | | | | |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 16 | 5-fluoro-1-(3-furoyl)-1′- | 5.02 | C26H25FN4O2 | 444.2 | 444.1956 | 1.04 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 17 | 5-fluoro-1-isobutyryl-1′- | 3.31 | C25H29FN4O | 420.23 | 420.2319 | 1.03 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 18 | 5-fluoro-1′-[(1-methyl-1H- | 2.45 | C25H25FN6OS | 476.18 | 476.179 | 1.07 | Z |
| | benzimidazol-2- | | | | | | |
| | yl)methyl]-1-[(4-methyl- | | | | | | |
| | 1,2,3-thiadiazol-5- | | | | | | |
| | yl)carbonyl]-1,2- | | | | | | |
| | dihydrospiro[indole-3,4′- | | | | | | |
| | piperidine] | | | | | | |
| 19 | 1-acetyl-5-fluoro-1′-[(1- | 4.79 | C23H25FN4O | 392.2 | 392.2007 | 0.91 | Z |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 20 | 5-fluoro-1′-[(1-methyl-1H- | 3.39 | C24H27FN4O | 406.22 | 406.2163 | 0.97 | Z |
| | benzimidazol-2- | | | | | | |
| | yl)methyl]-1-propionyl- | | | | | | |
| | 1,2-dihydrospiro[indole- | | | | | | |
| | 3,4′-piperidine] | | | | | | |
| 21 | 5-methyl-1′-[(1-methyl- | 1.16 | C26H28N6OS | 472.2 | 472.204 | 1.07 | Z |
| | 1H-benzimidazol-2- | | | | | | |
| | yl)methyl]-1-[(4-methyl- | | | | | | |
| | 1,2,3-thiadiazol-5- | | | | | | |
| | yl)carbonyl]-1,2- | | | | | | |
| | dihydrospiro[indole-3,4′- | | | | | | |
| | piperidine] | | | | | | |
| 22 | 1-acetyl-5-methyl-1′-[(1- | 3.83 | C24H28N4O | 388.23 | 388.2257 | 0.92 | Z |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 23 | 5-methyl-1′-[(1-methyl- | 0.926 | C25H30N4O | 402.24 | 402.2413 | 0.98 | Z |
| | 1H-benzimidazol-2- | | | | | | |
| | yl)methyl]-1-propionyl- | | | | | | |
| | 1,2-dihydrospiro[indole- | | | | | | |
| | 3,4′-piperidine] | | | | | | |
| 24 | 1-isobutyryl-5-methyl-1′- | 1.62 | C26H32N4O | 416.26 | 416.2569 | 1.04 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 25 | 5-chloro-1′-[(1-methyl- | 1.35 | C25H25ClN6OS | 492.15 | 492.1495 | 0.96 | Z |
| | 1H-benzimidazol-2- | | | | | | |
| | yl)methyl]-1-[(4-methyl- | | | | | | |
| | 1,2,3-thiadiazol-5- | | | | | | |
| | yl)carbonyl]-1,2- | | | | | | |
| | dihydrospiro[indole-3,4′- | | | | | | |
| | piperidine] | | | | | | |
| 26 | 1-acetyl-5-chloro-1′-[(1- | 2.45 | C23H25ClN4O | 408.17 | 408.1712 | 0.82 | Z |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 27 | 5-chloro-1′-[(1-methyl- | 1.06 | C24H27ClN4O | 422.19 | 422.1868 | 0.89 | Z |
| | 1H-benzimidazol-2- | | | | | | |
| | yl)methyl]-1-propionyl- | | | | | | |
| | 1,2-dihydrospiro[indole- | | | | | | |
| | 3,4′-piperidine] | | | | | | |
| 28 | 1′-[(1-methyl-1H- | 2.11 | C25H26N6OS | 458.19 | 458.1884 | 0.85 | Z |
| | benzimidazol-2- | | | | | | |
| | yl)methyl]-1-[(4-methyl- | | | | | | |
| | 1,2,3-thiadiazol-5- | | | | | | |
| | yl)carbonyl]-1,2- | | | | | | |
| | dihydrospiro[indole-3,4′- | | | | | | |
| | piperidine] | | | | | | |
| 29 | 1-(3-furoyl)-5-methyl-1′- | 2.43 | C27H28N4O2 | 440.22 | 440.2206 | 1.02 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 30 | methyl 6-{5-methyl-1′-[(1- | 4.89 | C29H36N4O3 | 488.28 | 488.2779 | 1.04 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 6-oxohexanoate | | | | | | |
| 31 | 1-[(3,5-dimethylisoxazol- | 2.04 | C28H31N5O2 | 469.25 | 469.2471 | 1.01 | Z |
| | 4-yl)carbonyl]-5-methyl- | | | | | | |
| | 1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 32 | methyl 5-{5-methyl-1′-[(1- | 1.85 | C28H34N4O3 | 474.26 | 474.2623 | 1.01 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 5-oxopentanoate | | | | | | |
| 33 | ethyl 4-{5-methyl-1′-[(1- | 1.35 | C28H34N4O3 | 474.26 | 474.2623 | 1.04 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 4-oxobutanoate | | | | | | |
| 34 | methyl 4-{5-methyl-1′-[(1- | 3.15 | C27H32N4O3 | 460.25 | 460.2467 | 0.99 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 4-oxobutanoate | | | | | | |
| 35 | 5-methyl-1′-[(1-methyl- | 3.61 | C28H29N5O | 451.24 | 451.2366 | 0.88 | Z |
| | 1H-benzimidazol-2- | | | | | | |
| | yl)methyl]-1-(pyridin-3- | | | | | | |
| | ylcarbonyl)-1,2- | | | | | | |
| | dihydrospiro[indole-3,4′- | | | | | | |
| | piperidine] | | | | | | |
| 36 | 1-(cyclopropylcarbonyl)- | 2.33 | C26H30N4O | 414.24 | 414.2413 | 0.99 | Z |
| | 5-methyl-1′-[(1-methyl- | | | | | | |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 37 | 1-(2-furoyl)-5-methyl-1′- | 1.82 | C27H28N4O2 | 440.22 | 440.2206 | 1 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 38 | 1-butyryl-5-methyl-1′-[(1- | 0.388 | C26H32N4O | 416.26 | 416.2569 | 1.02 | Z |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 39 | 5-chloro-1-[(3,5- | 2.83 | C27H28ClN5O2 | 489.19 | 489.1926 | 1.1 | Z |
| | dimethylisoxazol-4- | | | | | | |
| | yl)carbonyl]-1′-[(1- | | | | | | |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 40 | 2-{5-chloro-1′-[(1-methyl- | >4.43 | C25H27ClN4O3 | 466.18 | 466.1766 | 1.04 | Z |
| | 1H-benzimidazol-2- | | | | | | |
| | yl)methyl]spiro[indole- | | | | | | |
| | 3,4′-piperidin]-1(2H)-yl}- | | | | | | |
| | 2-oxoethyl acetate | | | | | | |
| 41 | ethyl 4-{5-chloro-1′-[(1- | 1.63 | C27H31ClN4O3 | 494.21 | 494.2078 | 1.12 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 4-oxobutanoate | | | | | | |
| 42 | methyl 4-{5-chloro-1′-[(1- | 3.23 | C26H29ClN4O3 | 480.19 | 480.1922 | 1.07 | Z |
| | methyl-1H-benzimidazol- | | | | | | |
| | 2-yl)methyl]spiro[indole- | | | | | | |
| | 3,4′-piperidin]-1(2H)-yl}- | | | | | | |
| | 4-oxobutanoate | | | | | | |
| 43 | 2-{5-chloro-1′-[(1-methyl- | 3.41 | C27H31ClN4O3 | 494.21 | 494.2078 | 1.13 | Z |
| | 1H-benzimidazol-2- | | | | | | |
| | yl)methyl]spiro[indole- | | | | | | |
| | 3,4′-piperidin]-1(2H)-yl}- | | | | | | |
| | 1,1-dimethyl-2-oxoethyl | | | | | | |
| | acetate | | | | | | |
| 44 | 5-chloro-1- | >5.10 | C24H27ClN4O2 | 438.18 | 438.1817 | 0.99 | Z |
| | (methoxyacetyl)-1′-[(1- | | | | | | |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 45 | 5-chloro-1- | 1.12 | C25H27ClN4O | 434.19 | 434.1868 | 1.08 | Z |
| | (cyclopropylcarbonyl)-1′- | | | | | | |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 46 | 5-chloro-1-(2-furoyl)-1′- | >4.86 | C26H25ClN4O2 | 460.17 | 460.1661 | 1.09 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 47 | 2-[4-(2-Methoxy-phenyl)- | 0.0771 | C21H25N3O | 335.2 | 336.2 | | K |
| | piperidin-1-ylmethyl]-1- | | | | | | |
| | methyl-1H- | | | | | | |
| | benzoimidazole | | | | | | |
| 48 | 1′-[(1-methyl-1H- | >6.63 | C30H28N6O | 488.23 | 488.2319 | 0.94 | Z |
| | benzimidazol-2- | | | | | | |
| | yl)methyl]-1-(quinoxalin- | | | | | | |
| | 6-ylcarbonyl)-1,2- | | | | | | |
| | dihydrospiro[indole-3,4′- | | | | | | |
| | piperidine] | | | | | | |
| 49 | 1′-[(1-methyl-1H- | 1.2 | C26H26N4OS | 442.18 | 442.1822 | 1.03 | Z |
| | benzimidazol-2- | | | | | | |
| | yl)methyl]-1-(3- | | | | | | |
| | thienylcarbonyl)-1,2- | | | | | | |
| | dihydrospiro[indole-3,4′- | | | | | | |
| | piperidine] | | | | | | |
| 50 | 1-(3-furoyl)-1′-[(1-methyl- | 4.93 | C26H26N4O2 | 426.21 | 426.205 | 0.96 | Z |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 51 | methyl 6-{1′-[(1-methyl- | 7.13 | C26H34N4O3 | 474.26 | 474.2623 | 1.03 | Z |
| | 1H-benzimidazol-2- | | | | | | |
| | yl)methyl]spiro[indole- | | | | | | |
| | 3,4′-piperidin]-1(2H)-yl}- | | | | | | |
| | 6-oxohexanoate | | | | | | |
| 52 | 1-(2,3-dihydro-1,4- | 1.39 | C30H30N4O3 | 494.23 | 494.2311 | 1.15 | Z |
| | benzodioxin-2- | | | | | | |
| | ylcarbonyl)-1′-[(1-methyl- | | | | | | |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 53 | 1-[(1,3-dimethyl-1H- | >6.29 | C27H30N6O | 454.25 | 454.2475 | 0.98 | Z |
| | pyrazol-5-yl)carbonyl]-1′- | | | | | | |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 54 | 1-[(3,5-dimethylisoxazol- | >5.56 | C27H29N5O2 | 455.23 | 455.2315 | 1 | Z |
| | 4-yl)carbonyl]-1′-[(1- | | | | | | |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 55 | 3-({1′-[(1-methyl-1H- | 3.01 | C29H27N5O | 461.22 | 461.221 | 1.06 | Z |
| | benzimidazol-2- | | | | | | |
| | yl)methyl]spiro[indole- | | | | | | |
| | 3,4′-piperidin]-1(2H)- | | | | | | |
| | yl}carbonyl)benzonitrile | | | | | | |
| 56 | 4-({1′-[(1-methyl-1H- | 1.75 | C29H27N5O | 461.22 | 461.221 | 1.05 | Z |
| | benzimidazol-2- | | | | | | |
| | yl)methyl]spiro[indole- | | | | | | |
| | 3,4′-piperidin]-1(2H)- | | | | | | |
| | yl}carbonyl)benzonitrile | | | | | | |
| 57 | 1′-[(1-methyl-1H- | 0.508 | C29H30N4O2 | 466.24 | 466.2362 | 1.1 | Z |
| | benzimidazol-2- | | | | | | |
| | yl)methyl]-1- | | | | | | |
| | (phenoxyacetyl)-1,2- | | | | | | |
| | dihydrospiro[indole-3,4′- | | | | | | |
| | piperidine] | | | | | | |
| 58 | 1-[(benzyloxy)acetyl]-1′- | 0.589 | C30H32N4O2 | 480.25 | 480.2518 | 1.06 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 59 | 1′-[(1-methyl-1H- | 3.11 | C26H26N4OS | 442.18 | 442.1822 | 1 | Z |
| | benzimidazol-2- | | | | | | |
| | yl)methyl]-1-(2- | | | | | | |
| | thienylcarbonyl)-1,2- | | | | | | |
| | dihydrospiro[indole-3,4′- | | | | | | |
| | piperidine] | | | | | | |
| 60 | 1-(cyclobutylcarbonyl)-1′- | 2.22 | C26H30N4O | 414.24 | 414.2413 | 0.99 | Z |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 61 | 1-(cyclopropylcarbonyl)- | 4.01 | C25H28N4O | 400.23 | 400.2257 | 0.93 | Z |
| | 1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 62 | 1′-[(1-methyl-1H- | 0.261 | C26H32N4O | 416.26 | 416.2569 | 1.03 | Z |
| | benzimidazol-2- | | | | | | |
| | yl)methyl]-1-pentanoyl- | | | | | | |
| | 1,2-dihydrospiro[indole- | | | | | | |
| | 3,4′-piperidine] | | | | | | |
| 63 | 1-(2-furoyl)-1′-[(1-methyl- | 4.51 | C26H26N4O2 | 426.21 | 426.205 | 0.94 | Z |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 64 | 1-butyryl-1′-[(1-methyl- | 1.41 | C25H30N4O | 402.24 | 402.2413 | 0.97 | Z |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 65 | 2-[4-(3-Methoxy-phenyl)- | 0.241 | C21H25N3O | 335.2 | 336.3 | | J |
| | piperidin-1-ylmethyl]-1- | | | | | | |
| | methyl-1H- | | | | | | |
| | benzoimidazole | | | | | | |
| 66 | 1-[1-(1-Methyl-1H- | >5.23 | C22H25N3O | 347.2 | 348.2 | 209 | J |
| | benzoimidazole-2- | | | | | | |
| | ylmethyl)-4-phenyl- | | | | | | |
| | piperidin-4-yl]-ethanone | | | | | | |
| 67 | (4aS,10bS)-3-(1-Methyl- | 0.693 | C22H25N3 | 331.2 | 332.205 | 2.29 | J |
| | 1H-benzoimidazol-2- | | | | | | |
| | ylmethyl)- | | | | | | |
| | 1,2,3,4,4a,5,6,10b- | | | | | | |
| | octahydro- | | | | | | |
| | benzo[f]isoquinoline | | | | | | |
| 68 | 4-(3,5-Dibromo-phenyl)- | 1.24 | C20H21Br2N3O | 477.01 | 478.005 | 2.34 | J |
| | 1-[(1-methyl-1H- | | | | | | |
| | benzoimidazol-2- | | | | | | |
| | yl)methyl]-piperidin-4-ol | | | | | | |
| 69 | 5-methoxy-1′-[(1-methyl- | >6.97 | C24H29N3O | 375.23 | 376.231 | 2.38 | J |
| | 1H-benzimidazol-2- | | | | | | |
| | yl)methyl]-3,4-dihydro- | | | | | | |
| | 2H-spiro[naphthalene- | | | | | | |
| | 1,4′-piperidine] | | | | | | |
| 70 | 6-metoxy-1′-[(1-methyl- | 2.68 | C24H29N3O | 375.23 | 376.231 | 2.3 | J |
| | 1H-benzimidazol-2- | | | | | | |
| | yl)methyl]-3,4-dihydro- | | | | | | |
| | 2H-spiro[naphthalene- | | | | | | |
| | 1,4′-piperidine] | | | | | | |
| 71 | 4-(4-Chloro-3- | 4.35 | C21H21CIF3N3O | 423.13 | 424.133 | 2.33 | J |
| | trifluoromethyl-phenyl)-1- | | | | | | |
| | [(1-methyl-1H- | | | | | | |
| | benzoimidazol-2- | | | | | | |
| | yl)methyl]-piperidin-4-ol | | | | | | |
| 72 | 4-(3-Methoxy-phenyl)-1- | 10.6 | C22H24N4O | 360.2 | 361.195 | 2.33 | J |
| | (1-methyl-1H- | | | | | | |
| | benzoimidazol-2- | | | | | | |
| | ylmethyl)-piperidine-4- | | | | | | |
| | carbonitrile | | | | | | |
| 73 | 1-[1-(1-Methyl-1H- | 3.97 | C23H27N3O | 361.22 | 362.215 | 2.25 | J |
| | benzoimidazol-2- | | | | | | |
| | ylmethyl)-4-phenyl- | | | | | | |
| | piperidin-4-yl]-propan-1- | | | | | | |
| | one | | | | | | |
| 74 | 1-Methyl-2-(4-phenyl- | 0.212 | C20H23N3 | 305.19 | 306.189 | 2.09 | J |
| | piperidin-1-ylmethyl)-1H- | | | | | | |
| | benzoimidazole | | | | | | |
| 75 | 4-(4-Bromo-phenyl)-1-(1- | >7.48 | C20H22BrN3O | 399.09 | 400.095 | 2.09 | J |
| | methyl-1H- | | | | | | |
| | benzoimidazol-2- | | | | | | |
| | ylmethyl)-piperidin-4-ol | | | | | | |
| 76 | 4-(4-Chloro-phenyl)-1-(1- | >5.73 | C20H22ClN3O | 355.15 | 356.145 | 2.04 | J |
| | methyl-1H- | | | | | | |
| | benzoimidazol-2- | | | | | | |
| | ylmethyl)-piperidin-4-ol | | | | | | |
| 77 | 1-(1-Methyl-1H- | >4.71 | C21H22N4 | 330.18 | 331.184 | 2.3 | J |
| | benzoimidazol-2- | | | | | | |
| | ylmethyl)-4-phenyl- | | | | | | |
| | piperidine-4-carbonitrile | | | | | | |
| 78 | 2-[4-(2-Isopropoxy- | 0.475 | C23H29N3O | 363.23 | 364.231 | 2.41 | J |
| | phenyl)-piperidin-1- | | | | | | |
| | ylmethyl]-1-methyl-1H- | | | | | | |
| | benzoimidazole | | | | | | |
| 79 | 2-[4-(2-Fluoro-phenyl)- | 0.021 | C20H22FN3 | 323.18 | 324.1 | | K |
| | piperidin-1-ylmethyl]-1- | | | | | | |
| | methyl-1H- | | | | | | |
| | benzoimidazole | | | | | | |
| 80 | N-{3-[(3S,4S)-3,4- | >1.25 | C23H30N4O2S | 426.21 | 427.209 | 2.09 | J |
| | Dimethyl-1-(1-methyl- | | | | | | |
| | 1H-benzoimidazol-2- | | | | | | |
| | ylmethyl)-piperidin-4-yl]- | | | | | | |
| | phenyl}- | | | | | | |
| | methanesulfonamide | | | | | | |
| 81 | 3-[(3R,4R)-3,4-Dimethyl- | 6.08 | C24H29N3O2 | 391.23 | 392.3 | 3.14 | J |
| | 1-(1-methyl-1H- | | | | | | |
| | benzoimidazol-2- | | | | | | |
| | ylmethyl)-piperidin-4-yl]- | | | | | | |
| | benzoic acid methyl | | | | | | |
| | ester | | | | | | |
| 82 | 3,5-Dimethyl-1-(1- | >7.44 | C22H27N3O | 349.22 | 350.215 | 2.06 | J |
| | methyl-1H- | | | | | | |
| | benzoimidazol-2- | | | | | | |
| | ylmethyl)-4-phenyl- | | | | | | |
| | piperidin-4-ol | | | | | | |
| 83 | 2-{4-[3-((2S,6R)-2,6- | 0.82 | C26H34N4O | 418.27 | 419.3 | | J |
| | Dimethyl-morpholin-4- | | | | | | |
| | yl)-phenyl]-piperidin-1- | | | | | | |
| | ylmethyl}-1-methyl-1H- | | | | | | |
| | benzoimidazole | | | | | | |
| 84 | 2-[4-(2- | 0.744 | C25H31N3O | 389.25 | 390.2 | 2.95 | W |
| | Cyclobutylmethoxy- | | | | | | |
| | phenyl)-piperidin-1- | | | | | | |
| | ylmethyl]-1-methyl-1H- | | | | | | |
| | benzoimidazole | | | | | | |
| 85 | 2-(4-{2-[2-(2,6-Dimethyl- | 1.37 | C28H38N4O2 | 462.3 | 463.2 | 1.94 | W |
| | morpholin-4-yl)-ethoxy]- | | | | | | |
| | phenyl}-piperidin-1- | | | | | | |
| | ylmethyl)-1-methyl-1H- | | | | | | |
| | benzoimidazole | | | | | | |
| 86 | 2-[4-(2- | 2.31 | C26H33N3O | 403.26 | 404.2 | 3.17 | W |
| | Cyclopentylmethoxy- | | | | | | |
| | phenyl)-piperidin-1- | | | | | | |
| | ylmethyl]-1-methyl-1H- | | | | | | |
| | benzoimidazole | | | | | | |
| 87 | 2-{4-[2-(2-Isopropoxy- | 0.621 | C25H33N3O2 | 407.26 | 408.2 | 2.26 | W |
| | ethoxy)-phenyl]- | | | | | | |
| | piperidin-1-ylmethyl}-1- | | | | | | |
| | methyl-1H- | | | | | | |
| | benzoimidazole | | | | | | |
| 88 | Dimethyl-(3-{2-[1-(1- | 4.19 | C25H34N4O | 406.27 | 407.3 | 1.94 | W |
| | methyl-1H- | | | | | | |
| | benzoimidazol-2- | | | | | | |
| | ylmethyl)-piperidin-4-yl]- | | | | | | |
| | phenoxy}-propyl)-amine | | | | | | |
| 89 | 2-{4-[2-(2-Ethoxy- | 1.75 | C24H31N3O2 | 393.24 | 394.2 | 2.06 | W |
| | ethoxy)-phenyl]- | | | | | | |
| | piperidin-1-ylmethyl}-1- | | | | | | |
| | methyl-1H- | | | | | | |
| | benzoimidazole | | | | | | |
| 90 | 1-Methyl-2-{4-[2-(2- | 5.73 | C26H34N4O2 | 434.27 | 435.2 | 2.69 | W |
| | morpholin-4-yl-ethoxy)- | | | | | | |
| | phenyl]-piperidin-1- | | | | | | |
| | ylmethyl}-1H- | | | | | | |
| | benzoimidazole | | | | | | |
| 91 | 2-{4-[2-(3-Ethoxy- | 1.4 | C25H33N3O2 | 407.26 | 408.2 | 2.3 | W |
| | propoxy)-phenyl]- | | | | | | |
| | piperidin-1-ylmethyl}-1- | | | | | | |
| | methyl-1H- | | | | | | |
| | benzoimidazole | | | | | | |
| 92 | 2-{4-[2-(2-Cyclopropyl- | >3.27 | C25H31N3O | 389.25 | 390.2 | 2.83 | W |
| | ethoxy)-phenyl]- | | | | | | |
| | piperidin-1-ylmethyl}-1- | | | | | | |
| | methyl-1H- | | | | | | |
| | benzoimidazole | | | | | | |
| 93 | 1-Methyl-2-[4-(2- | 0.324 | C25H33N3O | 391.26 | 392.2 | 3.08 | W |
| | pentyloxy-phenyl)- | | | | | | |
| | piperidin-1-ylmethyl]-1H- | | | | | | |
| | benzoimidazole | | | | | | |
| 94 | 1-Methyl-2-{4-[2-((S)-2- | 1.61 | C25H33N3O | 391.26 | 392.2 | 3.09 | W |
| | methyl-butoxy)-phenyl]- | | | | | | |
| | piperidin-1-ylmethyl}-1H- | | | | | | |
| | benzoimidazole | | | | | | |
| 95 | 2-[4-(2-Butoxy-phenyl)- | 1.04 | C24H31N3O | 377.25 | 378.2 | 2.85 | W |
| | piperidin-1-ylmethyl]-1- | | | | | | |
| | methyl-1H- | | | | | | |
| | benzoimidazole | | | | | | |
| 96 | 2-[4-(2-Isobutoxy- | >3.92 | C24H31N3O | 377.25 | 378.2 | 2.86 | W |
| | phenyl)-piperidin-1- | | | | | | |
| | ylmethyl]-1-methyl-1H- | | | | | | |
| | benzoimidazole | | | | | | |
| 97 | 1-Methyl-2-{4-[2-(2- | >9.64 | C27H30N4O | 426.24 | 427.2 | 2.88 | W |
| | pyridin-2-yl-ethoxy)- | | | | | | |
| | phenyl]-piperidin-1- | | | | | | |
| | ylmethyl}-1H- | | | | | | |
| | benzoimidazole | | | | | | |
| 98 | 1-Methyl-2-{4-[2-((S)-1- | >4.81 | C26H34N4O | 418.27 | 419.2 | 2 | W |
| | methyl-pyrrolidin-2- | | | | | | |
| | ylmethoxy)-phenyl]- | | | | | | |
| | piperidin-1-ylmethyl}-1H- | | | | | | |
| | benzoimidazole | | | | | | |
| 99 | 1-Methyl-2-{4-[2- | 2.12 | C25H31N3O2 | 405.24 | 406.2 | 1.94 | W |
| | (tetrahydro-pyran-4- | | | | | | |
| | yloxy)-phenyl]-piperidin- | | | | | | |
| | 1-ylmethyl}-1H- | | | | | | |
| | benzoimidazole | | | | | | |
| 100 | 2-[4-(2-Ethoxy-phenyl)- | 2.91 | C22H27N3O | 349.22 | 350.2 | 2.31 | W |
| | piperidin-1-ylmethyl]-1- | | | | | | |
| | methyl-1H- | | | | | | |
| | benzoimidazole | | | | | | |
| 101 | 1-Methyl-2-(4-{2-[(S)- | 5.72 | C24H29N3O2 | 391.23 | 392.2 | 2.9 | W |
| | (tetrahydro-furan-3- | | | | | | |
| | yl)oxy]-phenyl}-piperidin- | | | | | | |
| | 1-ylmethyl)-1H- | | | | | | |
| | benzoimidazole | | | | | | |
| 102 | 2-{4-[2-(Indan-2-yloxy)- | 1.5 | C29H31N3O | 437.25 | 438.2 | 2.82 | W |
| | phenyl]-piperidin-1- | | | | | | |
| | ylmethyl}-1-methyl-1H- | | | | | | |
| | benzoimidazole | | | | | | |
| 103 | 1-Methyl-2-{4-[2-(1- | >5.64 | C28H31N3O | 425.25 | 426.2 | 2.8 | W |
| | phenyl-ethoxy)-phenyl]- | | | | | | |
| | piperidin-1-ylmethyl}-1H- | | | | | | |
| | benzoimidazole | | | | | | |
| 104 | 5-bromo-1-methyl-1′-[(1- | 1.41 | C22H25BrN4 | 424.13 | 424.97 | 2.05 | Y |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 105 | 5-chloro-1-ethyl-1′-[(1- | 3.37 | C23H27ClN4 | 394.19 | 395.192 | 3.5 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 106 | 1-isobutyl-1′-[(1-methyl- | 2.58 | C25H32N4 | 388.26 | 389.263 | 3.79 | X |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 107 | 5-chloro-1′-[(1-methyl- | 6.65 | C26H31ClN4O | 450.22 | 451.219 | 3.43 | X |
| | 1H-benzimidazol-2- | | | | | | |
| | yl)methyl]-1- | | | | | | |
| | (tetrahydrofuran-2- | | | | | | |
| | ylmethyl)-1,2- | | | | | | |
| | dihydrospiro[indole-3,4′- | | | | | | |
| | piperidine] | | | | | | |
| 108 | 5-chloro-1′-[(1-methyl- | >8.73 | C27H28ClN5 | 457.2 | 458.203 | 2.53 | X |
| | 1H-benzimidazol-2- | | | | | | |
| | yl)methyl]-1-(pyridin-3- | | | | | | |
| | ylmethyl)-1,2- | | | | | | |
| | dihydrospiro[indole-3,4′- | | | | | | |
| | piperidine] | | | | | | |
| 109 | 1-benzyl-1′-[(1-methyl- | 5.22 | C28H30N4 | 422.25 | 423.247 | 3.82 | X |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 110 | 1-ethyl-1′-[(1-methyl-1H- | 4.98 | C23H28N4 | 360.23 | 361.231 | 2.69 | X |
| | benzimidazol-2- | | | | | | |
| | yl)methyl]-1,2- | | | | | | |
| | dihydrospiro[indole-3,4′- | | | | | | |
| | piperidine] | | | | | | |
| 111 | 1-(2,2-dimethylpropyl)-1′- | 10.3 | C26H34N4 | 402.28 | 403.278 | 2.51 | X |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 112 | 1′-[(1-methyl-1H- | >9.88 | C27H29N5 | 423.24 | 424.242 | 2.18 | X |
| | benzimidazol-2- | | | | | | |
| | yl)methyl]-1-(pyridin-3- | | | | | | |
| | ylmethyl)-1,2- | | | | | | |
| | dihydrospiro[indole-3,4′- | | | | | | |
| | piperidine] | | | | | | |
| 113 | 5-chloro-1-isobutyl-1′-[(1- | 4.37 | C25H31ClN4 | 422.22 | 423.224 | 2.55 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 114 | 1-ethyl-5-fluoro-1′-[(1- | 2.38 | C23H27FN4 | 378.22 | 379.222 | 2.88 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 115 | 5-fluoro-1-isobutyl-1′-[(1- | 6.49 | C25H31FN4 | 406.25 | 407.253 | 3.91 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 116 | 1′-[(1-methyl-1H- | 3.12 | C26H34N4 | 402.28 | 403.278 | 3.82 | X |
| | benzimidazol-2- | | | | | | |
| | yl)methyl]-1-(3- | | | | | | |
| | methylbutyl)-1,2- | | | | | | |
| | dihydrospiro[indole-3,4′- | | | | | | |
| | piperidine] | | | | | | |
| 117 | 5-fluoro-1′-[(1-methyl-1H- | >9.01 | C26H31FN4O | 434.25 | 435.248 | 3.18 | X |
| | benzimidazol-2- | | | | | | |
| | yl)methyl]-1- | | | | | | |
| | (tetrahydrofuran-2- | | | | | | |
| | ylmethyl)-1,2- | | | | | | |
| | dihydrospiro[indole-3,4′- | | | | | | |
| | piperidine] | | | | | | |
| 118 | 1-ethyl-5-methyl-1′-[(1- | 3.41 | C24H30N4 | 374.25 | 375.247 | 2.56 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 119 | 1-isobutyl-5-methyl-1′- | 2.9 | C26H34N4 | 402.28 | 403.278 | 2.36 | X |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 120 | 5-methyl-1′-[(1-methyl- | 2.7 | C27H36N4 | 416.29 | 417.294 | 2.25 | X |
| | 1H-benzimidazol-2- | | | | | | |
| | yl)methyl]-1-(3- | | | | | | |
| | methylbutyl)-1,2- | | | | | | |
| | dihydrospiro[indole-3,4′- | | | | | | |
| | piperidine] | | | | | | |
| 121 | 5,7-dimethyl-1′-[(1- | 1.15 | C28H38N4 | 430.31 | 431.31 | 2.94 | X |
| | methyl-1H-benzimidazol- | | | | | | |
| | 2-yl)methyl]-1-(3- | | | | | | |
| | methylbutyl)-1,2- | | | | | | |
| | dihydrospiro[indole-3,4′- | | | | | | |
| | piperidine] | | | | | | |
| 122 | 1-ethyl-5,7-dimethyl-1′- | 1.25 | C25H32N4 | 388.26 | 389.263 | 2.24 | X |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 123 | 5,7-dimethyl-1′-[(1- | 5.03 | C28H36N4O | 444.29 | 445.289 | 2.68 | X |
| | methyl-1H-benzimidazol- | | | | | | |
| | 2-yl)methyl]-1- | | | | | | |
| | (tetrahydrofuran-2- | | | | | | |
| | ylmethyl)-1,2- | | | | | | |
| | dihydrospiro[indole-3,4′- | | | | | | |
| | piperidine] | | | | | | |
| 124 | 5-methyl-1′-[(1-methyl- | 4.11 | C27H34N4O | 430.27 | 431.273 | 3.2 | X |
| | 1H-benzimidazol-2- | | | | | | |
| | yl)methyl]-1- | | | | | | |
| | (tetrahydrofuran-2- | | | | | | |
| | ylmethyl)-1,2- | | | | | | |
| | dihydrospiro[indole-3,4′- | | | | | | |
| | piperidine] | | | | | | |
| 125 | 1-isobutyl-5,7-dimethyl- | 3.93 | C27H36N4 | 416.29 | 417.294 | 2.8 | X |
| | 1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 126 | 1-(2-ethylbutyl)-5,7- | 6.01 | C29H40N4 | 444.33 | 445.325 | 3.29 | X |
| | dimethyl-1′-[(1-methyl- | | | | | | |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 127 | 1-butyl-5-chloro-1′-[(1- | 2.81 | C25H31ClN4 | 422.22 | 423.224 | 2.54 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 128 | 5-chloro-1′-[(1-methyl- | >8.98 | C21H23ClN4 | 366.16 | 367.161 | 2.42 | X |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 129 | 5-chloro-1- | 2.51 | C25H29ClN4 | 420.21 | 421.208 | 3.86 | X |
| | (cyclopropylmethyl)-1′- | | | | | | |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 130 | 1′-[(1-methyl-1H- | 3.08 | C24H30N4 | 374.25 | 375.247 | 3.24 | X |
| | benzimidazol-2- | | | | | | |
| | yl)methyl]-1-propyl-1,2- | | | | | | |
| | dihydrospiro[indole-3,4′- | | | | | | |
| | piperidine] | | | | | | |
| 131 | 5-chloro-1-methyl-1′-[(1- | 1.76 | C22H25ClN4 | 380.18 | 381.177 | 3.28 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 132 | 1-butyl-1′-[(1-methyl-1H- | 1.34 | C25H32N4 | 388.26 | 389.263 | 3.56 | X |
| | benzimidazol-2- | | | | | | |
| | yl)methyl]1,2- | | | | | | |
| | dihydrospiro[indole-3,4′- | | | | | | |
| | piperidine] | | | | | | |
| 133 | 1′-[(1-methyl-1H- | 6.16 | C26H34N4 | 402.28 | 403.278 | 2.48 | X |
| | benzimidazol-2- | | | | | | |
| | yl)methyl]-1-(2- | | | | | | |
| | methylbutyl)-1,2- | | | | | | |
| | dihydrospiro[indole-3,4′- | | | | | | |
| | piperidine] | | | | | | |
| 134 | 1-(cyclopropylmethyl)-1′- | 7.38 | C25H30N4 | 386.25 | 387.247 | 3.16 | X |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 135 | 1-methyl-1′-[(1-methyl- | 2.35 | C22H26N4 | 346.22 | 347.216 | 2.56 | X |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 136 | 5-fluoro-1-methyl-1′-[(1- | 2.65 | C22H25FN4 | 364.21 | 365.206 | 2.75 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 137 | 5-fluoro-1′-[(1-methyl-1H- | >7.89 | C24H29FN4 | 392.24 | 393.238 | 3.4 | X |
| | benzimidazol-2- | | | | | | |
| | yl)methyl]-1-propyl-1,2- | | | | | | |
| | dihydrospiro[indole-3,4′- | | | | | | |
| | piperidine] | | | | | | |
| 138 | 1-(cyclopropylmethyl)-5- | 8.59 | C25H29FN4 | 404.24 | 405.238 | 3.34 | X |
| | fluoro-1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 139 | 1-butyl-5-methyl-1′-[(1- | 1.97 | C26H34N4 | 402.28 | 403.278 | 3.39 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 140 | 1-butyl-5-fluoro-1′-[(1- | 2.12 | C25H31FN4 | 406.25 | 407.253 | 3.72 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 141 | 5-methyl-1′-[(1-methyl- | 5.67 | C27H36N4 | 416.29 | 417.294 | 2.53 | X |
| | 1H-benzimidazol-2- | | | | | | |
| | yl)methyl]-1-(2- | | | | | | |
| | methylbutyl)-1,2- | | | | | | |
| | dihydrospiro[indole-3,4′- | | | | | | |
| | piperidine] | | | | | | |
| 142 | 1-(cyclopropylmethyl)-5- | 3.02 | C26H32N4 | 400.26 | 401.263 | 2.98 | X |
| | methyl-1′-[(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 143 | 5-methyl-1′-[(1-methyl- | >8.67 | C22H26N4 | 346.22 | 347.216 | 1.96 | X |
| | 1H-benzimidazol-2- | | | | | | |
| | yl)methyl]-1,2- | | | | | | |
| | dihydrospiro[indole-3,4′- | | | | | | |
| | piperidine] | | | | | | |
| 144 | 5-methyl-1′-[(1-methyl- | 2.37 | C25H32N4 | 388.26 | 389.263 | 3.07 | X |
| | 1H-benzimidazol-2- | | | | | | |
| | yl)methyl]-1-propyl-1,2- | | | | | | |
| | dihydrospiro[indole-3,4′- | | | | | | |
| | piperidine] | | | | | | |
| 145 | 5,7-dimethyl-1′-[(1- | 2.69 | C26H34N4 | 402.28 | 403.278 | 2.48 | X |
| | methyl-1H-benzimidazol- | | | | | | |
| | 2-yl)methyl]-1-propyl-1,2- | | | | | | |
| | dihydrospiro[indole-3,4′- | | | | | | |
| | piperidine] | | | | | | |
| 146 | 1,5-dimethyl-1′-[(1- | 1.67 | C23H28N4 | 360.23 | 361.231 | 2.51 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 147 | 5,7-dimethyl-1′-[(1- | 5.81 | C23H28N4 | 360.23 | 361.231 | 2.12 | X |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 148 | 5,7-dimethyl-1′-[(1- | 4.73 | C28H36N4 | 430.31 | 431.31 | 3.06 | X |
| | methyl-1H-benzimidazol- | | | | | | |
| | 2-yl)methyl]-1-(2- | | | | | | |
| | methylbutyl)-1,2- | | | | | | |
| | dihydrospiro[indole-3,4′- | | | | | | |
| | piperidine] | | | | | | |
| 149 | 1-butyl-5,7-dimethyl-1′- | 1.64 | C27H36N4 | 416.29 | 417.294 | 2.72 | X |
| | [(1-methyl-1H- | | | | | | |
| | benzimidazol-2- | | | | | | |
| | yl)methyl]-1,2- | | | | | | |
| | dihydrospiro[indole-3,4′- | | | | | | |
| | piperidine] | | | | | | |
| 150 | 1-(cyclopropylmethyl)- | 3.27 | C27H34N4 | 414.28 | 415.278 | 2.45 | X |
| | 5,7-dimethyl-1′-[(1- | | | | | | |
| | methyl-1H-benzimidazol- | | | | | | |
| | 2-yl)methyl]-1,2- | | | | | | |
| | dihydrospiro[indole-3,4′- | | | | | | |
| | piperidine] | | | | | | |
| 151 | 1-Methyl-2-(3-phenyl- | 4.1 | C19H21N3 | 291.17 | 292.3 | 2.54 | J |
| | pyrrolidin-1-ylmethyl)- | | | | | | |
| | 1H-benzoimidazole | | | | | | |
| 152 | 2-[3-(2-Chloro-phenyl)- | 0.371 | C19H20ClN3 | 325.13 | 326.2 | 2.86 | J |
| | pyrrolidin-1-ylmethyl]-1- | | | | | | |
| | methyl-1H- | | | | | | |
| | benzoimidazole | | | | | | |
| 153 | (3R,4S)-1-(1-Methyl-1H- | 4.79 | C22H22F3N3O2 | 417.17 | 418.2 | 3.57 | J |
| | benzoimidazol-2- | | | | | | |
| | ylmethyl)-4-(4- | | | | | | |
| | trifluoromethyl-phenyl)- | | | | | | |
| | pyrrolidine-3-carboxylic | | | | | | |
| | acid methyl ester | | | | | | |
| 154 | 2-[3-(2,5-Dimethoxy- | 0.285 | C21H25N3O2 | 351.19 | 352.3 | 2.78 | J |
| | phenyl)-pyrrolidin-1- | | | | | | |
| | ylmethyl]-1-methyl-1H- | | | | | | |
| | benzoimidazole | | | | | | |
| 155 | 2-[3-(4-Fluoro-phenyl)- | 1.65 | C19H20FN3 | 309.16 | 310.3 | 2.69 | J |
| | pyrrolidin-1-ylmethyl]-1- | | | | | | |
| | methyl-1H- | | | | | | |
| | benzoimidazole | | | | | | |
| 156 | (3R,4S)-4-(4-Chloro- | 8.55 | C21H22ClN3O2 | 383.14 | 384.2 | 3.33 | J |
| | phenyl)-1-(1-methyl-1H- | | | | | | |
| | benzoimidazol-2- | | | | | | |
| | ylmethyl)-pyrrolidine-3- | | | | | | |
| | carboxylic acid methyl | | | | | | |
| | ester | | | | | | |
| 157 | 4-Bromo-2-[4-(2-fluoro- | 0.836 | C20H21BrFN3 | 401.09 | 404.1 | | J |
| | phenyl)-piperidin-1- | | | | | | |
| | ylmethyl]-1-methyl-1H- | | | | | | |
| | benzoimidazole | | | | | | |
| 158 | 2-(3-Benzyl-pyrrolidin-1- | 0.865 | C20H23N3 | 305.19 | 305.1887 | 1.29 | J |
| | ylmethyl)-1-methyl-1H- | | | | | | |
| | benzoimidazole | | | | | | |
| 159 | 4-Bromo-1-methy-2-[4- | 7.7 | C23H24BrN3O | 437.11 | 437.1097 | 1.09 | J |
| | (2-prop-2-ynyloxy- | | | | | | |
| | phenyl)-piperidin-1- | | | | | | |
| | ylmethyl]-1H- | | | | | | |
| | benzoimidazole | | | | | | |
| 160 | 4-Bromo-2-[4-(2-chloro- | 0.862 | C20H21BrClN3 | 417.06 | 418.061 | 2.5 | J |
| | phenyl)-piperidin-1- | | | | | | |
| | ylmethyl]-1-methyl-1H- | | | | | | |
| | benzoimidazole | | | | | | |
| 161 | 2-[4-(2-Chloro-phenyl)- | 0.144 | C20H22ClN3 | 339.15 | 340.135 | 1.38 | J |
| | piperidin-1-ylmethyl]-1- | | | | | | |
| | methyl-1H- | | | | | | |
| | benzoimidazole | | | | | | |
| 162 | 1-Methyl-2-[4-(3- | 0.254 | C21H22F3N3 | 373.18 | 374.081 | 1.53 | J |
| | trifluoromethyl-phenyl)- | | | | | | |
| | piperidin-1-ylmethyl]-1H- | | | | | | |
| | benzoimidazole | | | | | | |
| 163 | 2-[4-(2,5-Dimethoxy- | 0.928 | C22H27N3O2 | 365.21 | 366.115 | 1.28 | J |
| | phenyl)-piperidin-1- | | | | | | |
| | ylmethyl]-1-methyl-1H- | | | | | | |
| | benzoimidazole | | | | | | |
| 164 | 4-Bromo-1-methyl-2-[4- | 0.838 | C21H21BrF3N3 | 451.09 | 452.087 | 2.7 | J |
| | (4-trifluoromethyl- | | | | | | |
| | phenyl)-piperidin-1- | | | | | | |
| | ylmethyl]-1H- | | | | | | |
| | benzoimidazole | | | | | | |
| 165 | 4-Bromo-2-[4-(2,5- | 2.66 | C22H26BrN3O2 | 443.12 | 444.121 | 2.4 | J |
| | dimethoxy-phenyl)- | | | | | | |
| | piperidin-1-ylmethyl]-1- | | | | | | |
| | methyl-1H- | | | | | | |
| | benzoimidazole | | | | | | |
| 166 | 1-Methyl-2-[4-(4- | 0.0778 | C21H22F3N3 | 373.18 | 374.177 | 2.43 | K |
| | trifluoromethyl-phenyl)- | | | | | | |
| | piperidin-1-ylmethyl]-1H- | | | | | | |
| | benzoimidazole | | | | | | |
| 167 | 1-(1-Methyl-1H- | 4.38 | C21H22F3N3O | 389.17 | 390.073 | 1.37 | K |
| | benzoimidazol-2- | | | | | | |
| | ylmethyl)-4-(3- | | | | | | |
| | trifluoromethyl-phenyl)- | | | | | | |
| | piperidin-3-ol | | | | | | |
| 168 | (4aS,10bS)-3-[(4-Bromo- | 1.2 | C22H24BrN3 | 409.12 | 410.115 | 2.5 | K |
| | 1-methyl-1H- | | | | | | |
| | benzoimidazol-2- | | | | | | |
| | yl)methyl]- | | | | | | |
| | 1,2,3,4,4a,5,6,10b- | | | | | | |
| | octahydro- | | | | | | |
| | benzo[f]isoquinoline | | | | | | |
| 169 | 4-Bromo-2-[4-(2- | 1.23 | C21H24BrN3O | 413.11 | 414.11 | 2.41 | J |
| | methoxy-phenyl)- | | | | | | |
| | piperidin-1-ylmethyl]-1- | | | | | | |
| | methyl-1H- | | | | | | |
| | benzoimidazole | | | | | | |
| 170 | 8-{1-[(4-Bromo-1-methyl- | 8.09 | C25H25BrN4O | 476.12 | 477.121 | 2.12 | J |
| | 1H-benzoimidazol-2- | | | | | | |
| | yl)methyl]-piperidin-4-yl}- | | | | | | |
| | naphthalene-2-carboxylic | | | | | | |
| | acid amide | | | | | | |
| 171 | (4aR,10bS)-7-Methoxy- | 7.55 | C23H27N3O | 361.22 | 362.123 | 1.35 | J |
| | 3-(1-methyl-1H- | | | | | | |
| | benzoimidazol-2- | | | | | | |
| | ylmethyl)- | | | | | | |
| | 1,2,3,4,4a,5,6,10b- | | | | | | |
| | octahydro- | | | | | | |
| | benzo[f]isoquinoline | | | | | | |
| 172 | 2-[4-(5-Chloro-2-fluoro- | 0.00828 | C20H21ClFN3 | 357.14 | 359 | 2.32 | J |
| | phenyl)-piperidin-1- | | | | | | |
| | ylmethyl]-1-methyl-1H- | | | | | | |
| | benzoimidazole | | | | | | |
| 173 | 2-{4-[5-Chloro-2-((S)-2- | 1.7 | C26H33ClFN3O | 457.23 | 457.2289 | 1.75 | W |
| | methyl-butoxy)-phenyl]- | | | | | | |
| | piperidin-1-ylmethyl}-1- | | | | | | |
| | (2-fluoro-ethyl)-1H- | | | | | | |
| | benzoimidazole | | | | | | |
| 174 | 2-{4-[5-Chloro-2-((S)-2- | 0.447 | C25H32ClN3O | 425.22 | 425.2227 | 1.77 | W |
| | methyl-butoxy)-phenyl]- | | | | | | |
| | piperidin-1-ylmethyl}-1- | | | | | | |
| | methyl-1H- | | | | | | |
| | benzoimidazole | | | | | | |
| 175 | 2-{4-[5-Chloro-2-((S)-2- | 8.76 | C25H31ClFN3O | 443.21 | 443.2133 | 1.8 | W |
| | methyl-butoxy)-phenyl]- | | | | | | |
| | piperidin-1-ylmethyl}-1- | | | | | | |
| | fluoromethyl-1H- | | | | | | |
| | benzoimidazole | | | | | | |
| 176 | 2-{4-[5-Chloro-2-((S)-2- | 1.34 | C27H36ClN3O2 | 469.25 | 469.2488 | 1.8 | W |
| | methyl-butoxy)-phenyl]- | | | | | | |
| | piperidin-1-ylmethyl}-1- | | | | | | |
| | (2-methoxy-ethyl)-1H- | | | | | | |
| | benzoimidazole | | | | | | |
| 177 | 2-[4-(5-Chloro-2- | 3.47 | C24H29ClFN3O | 429.2 | 429.1977 | 1.73 | W |
| | isobutoxy-phenyl)- | | | | | | |
| | piperidin-1-ylmethyl]-1- | | | | | | |
| | fluoromethyl-1H- | | | | | | |
| | benzoimidazole | | | | | | |
| 178 | 2-[4-(5-Chloro-2- | 0.499 | C24H30ClN3O | 411.21 | 411.2071 | 1.71 | W |
| | isobutoxy-phenyl)- | | | | | | |
| | piperidin-1-ylmethyl]-1- | | | | | | |
| | methyl-1H- | | | | | | |
| | benzoimidazole | | | | | | |
| 179 | 2-[4-(5-Chloro-2- | 1.74 | C26H32ClN3O | 437.22 | 437.2227 | 1.8 | W |
| | isobutoxy-phenyl)- | | | | | | |
| | piperidin-1-ylmethyl]-1- | | | | | | |
| | cyclopropyl-1H- | | | | | | |
| | benzoimidazole | | | | | | |
| 180 | 2-[4-(5-Chloro-2- | 3.02 | C26H34ClN3O2 | 455.23 | 455.2332 | 1.73 | W |
| | isobutoxy-phenyl)- | | | | | | |
| | piperidin-1-ylmethyl]-1- | | | | | | |
| | (2-methoxy-ethyl)-1H- | | | | | | |
| | benzoimidazole | | | | | | |
| 181 | 2-[4-(5-Chloro-2- | 2.11 | C25H31ClFN3O | 443.21 | 443.2133 | 1.69 | W |
| | isobutoxy-phenyl)- | | | | | | |
| | piperidin-1-ylmethyl]-1- | | | | | | |
| | (2-fluoro-ethyl)-1H- | | | | | | |
| | benzoimidazole | | | | | | |
| 182 | 2-[4-(2-Butoxy-5-chloro- | 0.355 | C24H29ClFN3O | 429.2 | 429.1977 | 1.74 | W |
| | phenyl)-piperidin-1- | | | | | | |
| | ylmethyl]-1-fluoromethyl- | | | | | | |
| | 1H-benzoimidazole | | | | | | |
| 183 | 2-[4-(2-Butoxy-5-chloro- | 1.52 | C26H32ClN3O | 437.22 | 437.2227 | 1.8 | W |
| | phenyl)-piperidin-1- | | | | | | |
| | ylmethyl]-1-cyclopropyl- | | | | | | |
| | 1H-benzoimidazole | | | | | | |
| 184 | 2-[4-(2-Butoxy-5-chloro- | 0.479 | C26H34ClN3O2 | 455.23 | 455.2332 | 1.74 | W |
| | phenyl)-piperidin-1- | | | | | | |
| | ylmethyl]-1-(2-methoxy- | | | | | | |
| | ethyl)-1H- | | | | | | |
| | benzoimidazole | | | | | | |
| 185 | 2-[4-(2-Butoxy-5-chloro- | 0.138 | C24H30ClN3O | 411.21 | 411.2071 | 1.71 | W |
| | phenyl)-piperidin-1- | | | | | | |
| | ylmethyl]-1-methyl-1H- | | | | | | |
| | benzoimidazole | | | | | | |
| 186 | 2-[4-(2-Butoxy-5-chloro- | 0.246 | C25H31ClFN3O | 443.21 | 443.2133 | 1.7 | W |
| | phenyl)-piperidin-1- | | | | | | |
| | ylmethyl]-1-(2-fluoro- | | | | | | |
| | ethyl)-1H- | | | | | | |
| | benzoimidazole | | | | | | |
| 187 | 2-{4-[5-Chloro-2-(2- | 0.777 | C27H32ClN3O | 449.22 | 449.2227 | 1.79 | W |
| | cyclopropyl-ethoxy)- | | | | | | |
| | phenyl]-piperidin-1- | | | | | | |
| | ylmethyl}-1-cyclopropyl- | | | | | | |
| | 1H-benzoimidazole | | | | | | |
| 188 | 2-{4-[5-Chloro-2-(2- | 0.566 | C25H29ClFN3O | 441.2 | 441.1977 | 1.72 | W |
| | cyclopropyl-ethoxy)- | | | | | | |
| | phenyl]-piperidin-1- | | | | | | |
| | ylmethyl}-1-fluoromethyl- | | | | | | |
| | 1H-benzoimidazole | | | | | | |
| 189 | 2-{4-[5-Chloro-2-(2- | 0.153 | C25H30ClN3O | 423.21 | 423.2071 | 1.69 | W |
| | cyclopropyl-ethoxy)- | | | | | | |
| | phenyl]-piperidin-1- | | | | | | |
| | ylmethyl}-1-methyl-1H- | | | | | | |
| | benzoimidazole | | | | | | |
| 190 | 2-{4-[5-Chloro-2-(2- | 0.141 | C26H31ClFN3O | 455.21 | 455.2133 | 1.68 | W |
| | cyclopropyl-ethoxy)- | | | | | | |
| | phenyl]-piperidin-1- | | | | | | |
| | ylmethyl}-1-(2-fluoro- | | | | | | |
| | ethyl)-1H- | | | | | | |
| | benzoimidazole | | | | | | |
| 191 | 2-{4-[5-Chloro-2-(2- | 0.136 | C27H34ClN3O2 | 467.23 | 467.2332 | 1.72 | W |
| | cyclopropyl-ethoxy)- | | | | | | |
| | phenyl]-piperidin-1- | | | | | | |
| | ylmethyl}-1-(2-methoxy- | | | | | | |
| | ethyl)-1H- | | | | | | |
| | benzoimidazole | | | | | | |
| 192 | 2-[4-(5-Chloro-2- | 2.26 | C27H32ClN3O | 449.22 | 449.2227 | 1.84 | W |
| | cyclobutylmethoxy- | | | | | | |
| | phenyl)-piperidin-1- | | | | | | |
| | ylmethyl]-1-cyclopropyl- | | | | | | |
| | 1H-benzoimidazole | | | | | | |
| 193 | 2-[4-(5-Chloro-2- | 2.34 | C25H29ClFN3O | 441.2 | 441.1977 | 1.77 | W |
| | cyclobutylmethoxy- | | | | | | |
| | phenyl)-piperidin-1- | | | | | | |
| | ylmethyl]-1-fluoromethyl- | | | | | | |
| | 1H-benzoimidazole | | | | | | |
| 194 | 2-[4-(5-Chloro-2- | 0.473 | C25H30ClN3O | 423.21 | 423.2071 | 1.74 | W |
| | cyclobutylmethoxy- | | | | | | |
| | phenyl)-piperidin-1- | | | | | | |
| | ylmethyl]-1-methyl-1H- | | | | | | |
| | benzoimidazole | | | | | | |
| 195 | 2-[4-(5-Chloro-2- | 1.39 | C26H31ClFN3O | 455.21 | 455.2133 | 1.72 | W |
| | cyclobutylmethoxy- | | | | | | |
| | phenyl)-piperidin-1- | | | | | | |
| | ylmethyl]-1-(2-fluoro- | | | | | | |
| | ethyl)-1H- | | | | | | |
| | benzoimidazole | | | | | | |
| 196 | 2-[4-(5-Chloro-2- | 0.719 | C27H34ClN3O2 | 467.23 | 467.2332 | 1.77 | W |
| | cyclobutylmethoxy- | | | | | | |
| | phenyl)-piperidin-1- | | | | | | |
| | ylmethyl]-1-(2-methoxy- | | | | | | |
| | ethyl)-1H- | | | | | | |
| | benzoimidazole | | | | | | |
| 197 | 2-{4-[5-Chloro-2-(2- | 3.26 | C25H29ClFN3O | 441.2 | 441.1977 | 1.72 | W |
| | methyl- | | | | | | |
| | cyclopropylmethoxy)- | | | | | | |
| | phenyl]-piperidin-1- | | | | | | |
| | ylmethyl}-1-fluoromethyl- | | | | | | |
| | 1H-benzoimidazole | | | | | | |
| 198 | 2-{4-[5-Chloro-2-(2- | 2.25 | C27H32ClN3O | 449.22 | 449.2227 | 1.79 | W |
| | methyl- | | | | | | |
| | cyclopropylmethoxy)- | | | | | | |
| | phenyl]-piperidin-1- | | | | | | |
| | ylmethyl}-1-cyclopropyl- | | | | | | |
| | 1H-benzoimidazole | | | | | | |
| 199 | 2-{4-[5-Chloro-2-(2- | 0.39 | C25H30ClN3O | 423.21 | 423.2071 | 1.69 | W |
| | methyl- | | | | | | |
| | cyclopropylmethoxy)- | | | | | | |
| | phenyl]-piperidin-1- | | | | | | |
| | ylmethyl}-1-methyl-1H- | | | | | | |
| | benzoimidazole | | | | | | |
| 200 | 2-{4-[5-Chloro-2-(2- | 1.55 | C26H31ClFN3O | 455.21 | 455.2133 | 1.68 | W |
| | methyl- | | | | | | |
| | cyclopropylmethoxy)- | | | | | | |
| | phenyl]-piperidin-1- | | | | | | |
| | ylmethyl}-1-(2-fluoro- | | | | | | |
| | ethyl)-1H- | | | | | | |
| | benzoimidazole | | | | | | |
| 201 | 2-{4-[5-Chloro-2-(2- | 0.623 | C27H34ClN3O2 | 467.23 | 467.2332 | 1.72 | W |
| | methyl- | | | | | | |
| | cyclopropylmethoxy)- | | | | | | |
| | phenyl]-piperidin-1- | | | | | | |
| | ylmethyl}-1-(2-methoxy- | | | | | | |
| | ethyl)-1H- | | | | | | |
| | benzoimidazole | | | | | | |
| 202 | 2-{4-[5-Chloro-2- | 2.88 | C27H32ClN3O2 | 465.22 | 465.2176 | 1.54 | W |
| | (tetrahydro-furan-3- | | | | | | |
| | ylmethoxy)-phenyl]- | | | | | | |
| | piperidin-1-ylmethyl}-1- | | | | | | |
| | cyclopropyl-1H- | | | | | | |
| | benzoimidazole | | | | | | |
| 203 | 2-{4-[5-Chloro-2- | 0.712 | C25H30ClN3O2 | 439.2 | 439.202 | 1.42 | W |
| | (tetrahydro-furan-3- | | | | | | |
| | ylmethoxy)-phenyl]- | | | | | | |
| | piperidin-1-ylmethyl}-1- | | | | | | |
| | methyl-1H- | | | | | | |
| | benzoimidazole | | | | | | |
| 204 | 2-{4-[5-Chloro-2- | 3.61 | C25H29ClFN3O2 | 457.19 | 457.1926 | 1.47 | W |
| | (tetrahydro-furan-3- | | | | | | |
| | ylmethoxy)-phenyl]- | | | | | | |
| | piperidin-1-ylmethyl}-1- | | | | | | |
| | fluoromethyl-1H- | | | | | | |
| | benzoimidazole | | | | | | |
| 205 | 2-{4-[5-Chloro-2- | 3.35 | C27H34ClN3O3 | 483.23 | 483.2281 | 1.46 | W |
| | (tetrahydro-furan-3- | | | | | | |
| | ylmethoxy)-phenyl]- | | | | | | |
| | piperidin-1-ylmethyl}-1- | | | | | | |
| | (2-methoxy-ethyl)-1H- | | | | | | |
| | benzoimidazole | | | | | | |
| 206 | 2-{4-[5-Chloro-2- | 2.6 | C27H32ClN3O2 | 465.22 | 465.2176 | 1.55 | W |
| | (tetrahydro-furan-2- | | | | | | |
| | ylmethoxy)-phenyl]- | | | | | | |
| | piperidin-1-ylmethyl}-1- | | | | | | |
| | cyclopropyl-1H- | | | | | | |
| | benzoimidazole | | | | | | |
| 207 | 2-{4-[5-Chloro-2- | 0.666 | C25H30ClN3O2 | 439.2 | 439.202 | 1.43 | W |
| | (tetrahydro-furan-2- | | | | | | |
| | ylmethoxy)-phenyl]- | | | | | | |
| | piperidin-1-ylmethyl}-1- | | | | | | |
| | methyl-1H- | | | | | | |
| | benzoimidazole | | | | | | |
| 208 | 2-{4-[5-Chloro-2- | 4.13 | C25H29ClFN3O2 | 457.19 | 457.1926 | 1.48 | W |
| | (tetrahydro-furan-2- | | | | | | |
| | ylmethoxy)-phenyl]- | | | | | | |
| | piperidin-1-ylmethyl}-1- | | | | | | |
| | fluoromethyl-1H- | | | | | | |
| | benzoimidazole | | | | | | |
| 209 | 2-{4-[5-Chloro-2- | 1.7 | C27H34ClN3O3 | 483.23 | 483.2281 | 1.49 | W |
| | (tetrahydro-furan-2- | | | | | | |
| | ylmethoxy)-phenyl]- | | | | | | |
| | piperidin-1-ylmethyl}-1- | | | | | | |
| | (2-methoxy-ethyl)-1H- | | | | | | |
| | benzoimidazole | | | | | | |
| 210 | 2-{4-[5-Chloro-2- | 1.04 | C26H31ClFN3O2 | 471.21 | 471.2082 | 1.45 | W |
| | (tetrahydro-furan-2- | | | | | | |
| | ylmethoxy)-phenyl]- | | | | | | |
| | piperidin-1-ylmethyl}-1- | | | | | | |
| | (2-fluoro-ethyl)-1H- | | | | | | |
| | benzoimidazole | | | | | | |
| 211 | 2-{4-[5-Chloro-2-(3- | 8.22 | C25H29ClFN3O2 | 457.19 | 457.1926 | 1.45 | W |
| | methyl-oxetan-3- | | | | | | |
| | ylmethoxy)-phenyl]- | | | | | | |
| | piperidin-1-ylmethyl}-1- | | | | | | |
| | fluoromethyl-1H- | | | | | | |
| | benzoimidazole | | | | | | |
| 212 | 2-{4-[5-Chloro-2-(3- | 10.5 | C26H31ClFN3O2 | 471.21 | 471.2082 | 1.42 | W |
| | methyl-oxetan-3- | | | | | | |
| | ylmethoxy)-phenyl]- | | | | | | |
| | piperidin-1-ylmethyl}-1- | | | | | | |
| | (2-fluoro-ethyl)-1H- | | | | | | |
| | benzoimidazole | | | | | | |
| 213 | 2-{4-[5-Chloro-2-(3- | 6.17 | C25H30ClN3O2 | 439.2 | 439.202 | 1.41 | W |
| | methyl-oxetan-3- | | | | | | |
| | ylmethoxy)-phenyl]- | | | | | | |
| | piperidin-1-ylmethyl}-1- | | | | | | |
| | methyl-1H- | | | | | | |
| | benzoimidazole | | | | | | |
| 214 | 2-{4-[5-Chloro-2- | 2.01 | C28H34ClN3O2 | 479.23 | 479.2332 | 1.67 | W |
| | (tetrahydro-pyran-2- | | | | | | |
| | ylmethoxy)-phenyl]- | | | | | | |
| | piperidin-1-ylmethyl}-1- | | | | | | |
| | cyclopropyl-1H- | | | | | | |
| | benzoimidazole | | | | | | |
| 215 | 2-{4-[5-Chloro-2- | 1.57 | C26H31ClFN3O2 | 471.21 | 471.2082 | 1.61 | W |
| | (tetrahydro-pyran-2- | | | | | | |
| | ylmethoxy)-phenyl]- | | | | | | |
| | piperidin-1-ylmethyl}-1- | | | | | | |
| | fluoromethyl-1H- | | | | | | |
| | benzoimidazole | | | | | | |
| 216 | 2-{4-[5-Chloro-2- | 0.534 | C26H32ClN3O2 | 453.22 | 453.2176 | 1.55 | W |
| | (tetrahydro-pyran-2- | | | | | | |
| | ylmethoxy)-phenyl]- | | | | | | |
| | piperidin-1-ylmethyl}-1- | | | | | | |
| | methyl-1H- | | | | | | |
| | benzoimidazole | | | | | | |
| 217 | 2-{4-[5-Chloro-2- | 1.18 | C27H33ClFN3O2 | 485.22 | 485.2238 | 1.56 | W |
| | (tetrahydro-pyran-2- | | | | | | |
| | ylmethoxy)-phenyl]- | | | | | | |
| | piperidin-1-ylmethyl}-1- | | | | | | |
| | (2-fluoro-ethyl)-1H- | | | | | | |
| | benzoimidazole | | | | | | |
| 218 | 2-{4-[5-Chloro-2- | 1.55 | C28H36ClN3O3 | 497.24 | 497.2437 | 1.6 | W |
| | (tetrahydro-pyran-2- | | | | | | |
| | ylmethoxy)-phenyl]- | | | | | | |
| | piperidin-1-ylmethyl}-1- | | | | | | |
| | (2-methoxy-ethyl)-1H- | | | | | | |
| | benzoimidazole | | | | | | |
| 219 | 2-[4-(5-Chloro-2- | 0.731 | C25H30ClN3O | 423.21 | 423.2071 | 1.72 | W |
| | propoxy-phenyl)- | | | | | | |
| | piperidin-1-ylmethyl]-1- | | | | | | |
| | cyclopropyl-1H- | | | | | | |
| | beozoimidazole | | | | | | |
| 220 | 2-[4-(5-Chloro-2- | 0.216 | C23H28ClN3O | 397.19 | 397.1915 | 1.62 | W |
| | propoxy-phenyl)- | | | | | | |
| | piperidin-1-ylmethyl]-1- | | | | | | |
| | methyl-1H- | | | | | | |
| | benzoimidazole | | | | | | |
| 221 | 2-[4-(5-Chloro-2- | 0.413 | C23H27ClFN3O | 415.18 | 415.1821 | 1.65 | W |
| | propoxy-phenyl)- | | | | | | |
| | piperidin-1-ylmethyl]-1- | | | | | | |
| | fluoromethyl-1H- | | | | | | |
| | benzoimidazole | | | | | | |
| 222 | 2-[4-(5-Chloro-2- | 0.248 | C24H29ClFN3O | 429.2 | 429.1977 | 1.62 | W |
| | propoxy-phenyl)- | | | | | | |
| | piperidin-1-ylmethyl]-1- | | | | | | |
| | (2-fluoro-ethyl)-1H- | | | | | | |
| | benzoimidazole | | | | | | |
| 223 | 2-[4-(5-Chloro-2- | 0.233 | C25H32ClN3O2 | 441.22 | 441.2176 | 1.66 | W |
| | propoxy-phenyl)- | | | | | | |
| | piperidin-1-ylmethyl]-1- | | | | | | |
| | (2-methoxy-ethyl)-1H- | | | | | | |
| | benzoimidazole | | | | | | |
| 224 | 2-[4-(5-Chloro-2-ethoxy- | 4.26 | C22H26ClN3O | 383.18 | 383.1759 | 1.55 | W |
| | phenyl)-piperidin-1- | | | | | | |
| | ylmethyl]-1-methyl-1H- | | | | | | |
| | benzoimidazole | | | | | | |
| 225 | 2-[4-(5-Chloro-2-ethoxy- | 0.999 | C24H28ClN3O | 409.19 | 409.1915 | 1.64 | W |
| | phenyl)-piperidin-1- | | | | | | |
| | ylmethyl]-1-cyclopropyl- | | | | | | |
| | 1H-benzoimidazole | | | | | | |
| 226 | 2-[4-(5-Chloro-2-ethoxy- | 0.637 | C22H25ClFN3O | 401.17 | 401.1665 | 1.57 | W |
| | phenyl)-piperidin-1- | | | | | | |
| | ylmethyl]-1-fluoromethyl- | | | | | | |
| | 1H-benzoimidazole | | | | | | |
| 227 | 2-[4-(5-Chloro-2-ethoxy- | 0.21 | C23H27ClFN3O | 415.18 | 415.1821 | 1.53 | W |
| | phenyl)-piperidin-1- | | | | | | |
| | ylmethyl]-1-(2-fluoro- | | | | | | |
| | ethyl)-1H- | | | | | | |
| | benzoimidazole | | | | | | |
| 228 | 2-[4-(5-Chloro-2-ethoxy- | 0.185 | C24H30ClN3O2 | 427.2 | 427.202 | 1.57 | W |
| | phenyl)-piperidin-1- | | | | | | |
| | ylmethyl]-1-(2-methoxy- | | | | | | |
| | ethyl)-1H- | | | | | | |
| | benzoimidazole | | | | | | |
| 229 | 2-[4-(5-Chloro-2- | 0.357 | C23H26ClN3O | 395.18 | 395.1759 | 1.16 | W |
| | methoxy-phenyl)- | | | | | | |
| | piperidin-1-ylmethyl]-1- | | | | | | |
| | cyclopropyl-1H- | | | | | | |
| | benzoimidazole | | | | | | |
| 230 | 2-[4-(5-Chloro-2- | 0.209 | C23H28ClN3O2 | 413.19 | 413.1864 | 1.13 | W |
| | methoxy-phenyl)- | | | | | | |
| | piperidin-1-ylmethyl]-1- | | | | | | |
| | (2-methoxy-ethyl)-1H- | | | | | | |
| | benzoimidazole | | | | | | |
| 231 | 2-[4-(5-Chloro-2- | 0.115 | C21H24ClN3O | 369.16 | 369.1603 | 1.06 | W |
| | methoxy-phenyl)- | | | | | | |
| | piperidin-1-ylmethyl]-1- | | | | | | |
| | methyl-1H- | | | | | | |
| | benzoimidazole | | | | | | |
| 232 | 2-[4-(5-Chloro-2- | 0.396 | C21H23ClFN3O | 387.15 | 387.1509 | 1.12 | W |
| | methoxy-phenyl)- | | | | | | |
| | piperidin-1-ylmethyl]-1- | | | | | | |
| | fluoromethyl-1H- | | | | | | |
| | benzoimidazole | | | | | | |
| 233 | 2-[4-(5-Chloro-2- | 0.264 | C22H25ClFN3O | 401.17 | 401.1665 | 1.13 | W |
| | methoxy-phenyl)- | | | | | | |
| | piperidin-1-ylmethyl]-1- | | | | | | |
| | (2-fluoro-ethyl)-1H- | | | | | | |
| | benzoimidazole | | | | | | |
| 234 | 4-Bromo-2-[4-(5-chloro- | 1.31 | C21H23BrClN3O | 447.07 | 447.0708 | 1.16 | W |
| | 2-methoxy-phenyl)- | | | | | | |
| | piperidin-1-ylmethyl]-1- | | | | | | |
| | methyl-1H- | | | | | | |
| | benzoimidazole | | | | | | |
| 235 | 2-[4-(5-Chloro-2-prop-2- | 0.258 | C25H26ClN3O | 419.18 | 419.1759 | 1.19 | W |
| | ynyloxy-phenyl)- | | | | | | |
| | piperidin-1-ylmethyl]-1- | | | | | | |
| | cyclopropyl-1H- | | | | | | |
| | benzoimidazole | | | | | | |
| 236 | 2-[4-(5-Chloro-2-prop-2- | 0.0741 | C24H25ClFN3O | 425.17 | 425.1665 | 1.15 | W |
| | ynyloxy-phenyl)- | | | | | | |
| | piperidin-1-ylmethyl]-1- | | | | | | |
| | (2-fluoro-ethyl)-1H- | | | | | | |
| | benzoimidazole | | | | | | |
| 237 | 2-[4-(5-Chloro-2-prop-2- | 0.302 | C23H23ClFN3O | 411.15 | 411.1509 | 1.15 | W |
| | ynyloxy-phenyl)- | | | | | | |
| | piperidin-1-ylmethyl]-1- | | | | | | |
| | fluoromethyl-1H- | | | | | | |
| | benzoimidazole | | | | | | |
| 238 | 2-[4-(5-Chloro-2-prop-2- | 0.0781 | C23H24ClN3O | 393.16 | 393.1603 | 1.1 | W |
| | ynyloxy-phenyl)- | | | | | | |
| | piperidin-1-ylmethyl]-1- | | | | | | |
| | methyl-1H- | | | | | | |
| | benzoimidazole | | | | | | |
| 239 | 2-[4-(5-Chloro-2-prop-2- | 0.079 | C25H28ClN3O2 | 437.19 | 437.1864 | 1.16 | W |
| | ynyloxy-phenyl)- | | | | | | |
| | piperidin-1-ylmethyl]-1- | | | | | | |
| | (2-methoxy-ethyl)-1H- | | | | | | |
| | benzoimidazole | | | | | | |
| 240 | 1-(2-Fluoro-ethyl)-2-[4- | 0.146 | C22H26FN3O | 367.21 | 368.206 | 2.77 | J |
| | (2-methoxy-phenyl)- | | | | | | |
| | piperidin-1-ylmethyl]-1H- | | | | | | |
| | benzoimidazole | | | | | | |
| 241 | 1-(2-Fluoro-ethyl)-2-[4- | 0.217 | C21H23F2N3 | 355.19 | 356.186 | 2.74 | J |
| | (2-fluoro-phenyl)- | | | | | | |
| | piperidin-1-ylmethyl]-1H- | | | | | | |
| | benzoimidazole | | | | | | |
| 242 | 1-(2-Fluoro-ethyl)-2-[4- | 0.301 | C22H23F4N3 | 405.18 | 406.183 | 3.15 | J |
| | (4-trifluoromethyl- | | | | | | |
| | phenyl)-piperidin-1- | | | | | | |
| | ylmethyl]-1H- | | | | | | |
| | benzoimidazole | | | | | | |
| 243 | 1-(2-Fluoro-ethyl)-2-[4- | 1.26 | C22H23F4N3 | 405.18 | 406.183 | 3.08 | J |
| | (2-trifluoromethyl- | | | | | | |
| | phenyl)-piperidin-1- | | | | | | |
| | ylmethyl]-1H- | | | | | | |
| | benzoimidazole | | | | | | |
| 244 | 1-Cyclopropyl-2-[4-(2- | 0.484 | C23H27N3O | 361.22 | 362.215 | 2.86 | J |
| | methoxy-phenyl)- | | | | | | |
| | piperidin-1-ylmethyl]-1H- | | | | | | |
| | benzoimidazole | | | | | | |
| 245 | 1-Cyclopropyl-2-[4-(2- | 3.35 | C23H24F3N3 | 399.19 | 400.192 | 3.16 | J |
| | trifluoromethyl-phenyl)- | | | | | | |
| | piperidin-1-ylmethyl]-1H- | | | | | | |
| | benzoimidazole | | | | | | |
| 246 | 1-(2-Methoxy-ethyl)-2-[4- | 0.418 | C23H26F3N3O | 417.2 | 418.203 | 3.15 | J |
| | (4-trifluoromethyl- | | | | | | |
| | phenyl)-piperidin-1- | | | | | | |
| | ylmethyl]-1H- | | | | | | |
| | benzoimidazole | | | | | | |
| 247 | 1-Cyclopropyl-2-[4-(4- | 0.821 | C23H24F3N3 | 399.19 | 400.192 | 3.22 | J |
| | trifluoromethyl-phenyl)- | | | | | | |
| | piperidin-1-ylmethyl]-1H- | | | | | | |
| | benzoimidazole | | | | | | |
| 248 | 1-(2-Methoxy-ethyl)-2-[4- | 0.32 | C23H29N3O2 | 379.23 | 380.226 | 2.76 | J |
| | (2-methoxy-phenyl)- | | | | | | |
| | piperidin-1-ylmethyl]-1H- | | | | | | |
| | benzoimidazole | | | | | | |
| 249 | 2-[4-(2-Fluoro-phenyl)- | 0.251 | C22H26FN3O | 367.21 | 368.206 | 2.71 | J |
| | piperidin-1-ylmethyl]-1- | | | | | | |
| | (2-methoxy-ethyl)-1H- | | | | | | |
| | benzoimidazole | | | | | | |
| 250 | 1-(2-Methoxy-ethyl)-2-[4- | 0.545 | C23H26F3N3O | 417.2 | 418.203 | 3.08 | J |
| | (2-trifluoromethyl- | | | | | | |
| | phenyl)-piperidin-1- | | | | | | |
| | ylmethyl]-1H- | | | | | | |
| | benzoimidazole | | | | | | |
| 251 | 1-Cyclopropyl-2-[4-(2- | 1.34 | C22H24FN3 | 349.2 | 350.195 | 2.83 | J |
| | fluoro-phenyl)-piperidin- | | | | | | |
| | 1-ylmethyl]-1H- | | | | | | |
| | benzoimidazole | | | | | | |
| 252 | 1-Fluoromethyl-2-[4-(2- | 0.225 | C20H21F2N3 | 341.17 | 342.1 | | J |
| | fluoro-phenyl)-piperidin- | | | | | | |
| | 1-ylmethyl]-1H- | | | | | | |
| | benzoimidazole | | | | | | |
| 253 | 1-Fluoromethyl-2-[4-(2- | 0.116 | C20H21F2N3 | 341.17 | 342.17 | 2.71 | J |
| | fluoro-phenyl)-piperidin- | | | | | | |
| | 1-ylmethyl]-1H- | | | | | | |
| | benzoimidazole | | | | | | |
| 254 | 1-Fluoromethyl-2-[4-(2- | 0.0624 | C21H24FN3O | 353.19 | 354.19 | 2.77 | J |
| | methoxy-phenyl)- | | | | | | |
| | piperidin-1-ylmethyl]-1H- | | | | | | |
| | benzoimidazole | | | | | | |
| 255 | 1-Fluoromethyl-2-[4-(4- | 0.164 | C21H21F4N3 | 391.17 | 392.167 | 3.14 | J |
| | trifluoromethyl-phenyl)- | | | | | | |
| | piperidin-1-ylmethyl]-1H- | | | | | | |
| | benzoimidazole | | | | | | |
| 256 | 1-Fluoromethyl-2-[4-(2- | 0.422 | C21H21F4N3 | 391.17 | 392.167 | 3.06 | J |
| | trifluoromethyl-phenyl)- | | | | | | |
| | piperidin-1-ylmethyl]-1H- | | | | | | |
| | benzoimidazole | | | | | | |
| 257 | 1-Methyl-2-[4-(2- | 0.109 | C21H22F3N3 | 373.18 | 374.177 | 2.87 | J |
| | trifluoromethyl-phenyl)- | | | | | | |
| | piperidin-1-ylmethyl]-1H- | | | | | | |
| | benzoimidazole | | | | | | |
| 258 | 5,6-Difluoro-2-[4-(2- | 0.0805 | C21H23F2N3O | 371.18 | 372.181 | 2.84 | J |
| | methoxy-phenyl)- | | | | | | |
| | piperidin-1-ylmethyl]-1- | | | | | | |
| | methyl-1H- | | | | | | |
| | benzoimidazole | | | | | | |
| 259 | 5,6-Difluoro-2-[4-(2- | 0.468 | C20H20F3N3 | 359.16 | 360.161 | 2.8 | J |
| | fluoro-phenyl)-piperidin- | | | | | | |
| | 1-ylmethyl]-1-methyl-1H- | | | | | | |
| | benzoimidazole | | | | | | |
| 260 | 5,6-Difluoro-1-methyl-2- | 0.422 | C21H20F5N3 | 409.16 | 410.158 | 3.19 | J |
| | [4-(4-trifluoromethyl- | | | | | | |
| | phenyl)-piperidin-1- | | | | | | |
| | ylmethyl]-1H- | | | | | | |
| | benzoimidazole | | | | | | |
| 261 | 5,6-Difluoro-1-methyl-2- | 2.31 | C21H20F5N3 | 409.16 | 410.158 | 3.12 | J |
| | [4-(2-trifluoromethyl- | | | | | | |
| | phenyl)-piperidin-1- | | | | | | |
| | ylmethyl]-1H- | | | | | | |
| | benzoimidazole | | | | | | |
| 262 | 1′-[(1-Methyl-1H- | 1.17 | C21H23N3O | 333.18 | 334.192 | 2.22 | J |
| | benzimidazol-2- | | | | | | |
| | yl)methyl]spiro[1- | | | | | | |
| | benzofuran-3,4′- | | | | | | |
| | piperidine] | | | | | | |
| 263 | 5-Bromo-2-[4-(2- | 0.242 | C21H24BrN3O | 413.11 | 416.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 264 | 2-[4-(2-Methoxy-phenyl)- | 1.64 | C21H26N4O | 350.21 | 351.2 | | R1 |
| | piperidin-1-ylmethyl]-1- | | | | | | |
| | methyl-1H- | | | | | | |
| | benzoimidazol-4-ylamine | | | | | | |
| 265 | 2-[1-(1-Methyl-1H- | 1.44 | C20H23N3O | 321.18 | 322 | | K |
| | benzoimidazol-2- | | | | | | |
| | ylmethyl)-piperidin-4-yl]- | | | | | | |
| | phenol | | | | | | |
| 266 | 2-[4-(2-Methoxy-phenyl)- | 3.35 | C22H24N4O | 360.2 | 361.1 | | O |
| | piperidin-1-ylmethyl]-1- | | | | | | |
| | methyl-1H- | | | | | | |
| | benzoimidazole-4- | | | | | | |
| | carbonitrile | | | | | | |
| 267 | 2-[4-(2-Fluoro-phenyl)- | 0.249 | C21H24FN3 | 337.2 | 338.1 | | R |
| | piperidin-1-ylmethyl]-1,4- | | | | | | |
| | dimethyl-1H- | | | | | | |
| | benzoimidazole | | | | | | |
| 268 | 2-[4-(2-Fluoro-phenyl)- | 1.71 | C21H21FN4 | 348.18 | 349.1 | | O |
| | piperidin-1-ylmethyl]-1- | | | | | | |
| | methyl-1H- | | | | | | |
| | benzoimidazole-4- | | | | | | |
| | carbonitrile | | | | | | |
| 269 | 5-Bromo-2-[4-(2-fluoro- | 0.445 | C20H21BrFN3 | 401.09 | 402.1 | | K |
| | phenyl)-piperidin-1- | | | | | | |
| | ylmethyl]-1-methyl-1H- | | | | | | |
| | benzoimidazole | | | | | | |
| 270 | 4-Chloro-2-[4-(2- | 0.838 | C21H24ClN3O | 369.16 | 370.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 271 | 4-Fluoro-2-[4-(2- | 0.0211 | C21H24FN3O | 353.19 | 354.1 | | M |
| | methoxy-phenyl)- | | | | | | |
| | piperidin-1-ylmethyl]-1- | | | | | | |
| | methyl-1H- | | | | | | |
| | benzoimidazole | | | | | | |
| 272 | 2-[4-(2-Fluoro-phenyl)- | 0.073 | C21H21FN4 | 348.17 | 349.2 | | K, M1 |
| | piperidin-1-ylmethyl]-1- | | | | | | |
| | methyl-1H- | | | | | | |
| | benzoimidazole-5- | | | | | | |
| | carbonitrile | | | | | | |
| 273 | 2-[4-(2-Fluoro-phenyl)- | 0.0797 | C21H24FN3 | 337.2 | 338.1 | | R |
| | piperidin-1-ylmethyl]-1,5- | | | | | | |
| | dimethyl-1H- | | | | | | |
| | benzoimidazole | | | | | | |
| 274 | 6-Bromo-2-[4-(2-fluoro- | 0.318 | C20H21BrFN3 | 401.09 | 402 | | K |
| | phenyl)-piperidin-1- | | | | | | |
| | ylmethyl]-1-methyl-1H- | | | | | | |
| | benzoimidazole | | | | | | |
| 275 | 2-[4-(2-Fluoro-phenyl)- | 0.211 | C21H21FN4 | 348.17 | 349.1 | | O |
| | piperidin-1-ylmethyl]-3- | | | | | | |
| | methyl-3H- | | | | | | |
| | benzoimidazole-5- | | | | | | |
| | carbonitrile | | | | | | |
| 276 | 2-[4-(2-Fluoro-phenyl)- | 0.204 | C26H26FN3 | 399.21 | 400.2 | | P |
| | piperidin-1-ylmethyl]-1- | | | | | | |
| | methyl-6-phenyl-1H- | | | | | | |
| | benzoimidazole | | | | | | |
| 277 | 2-[4-(2-Fluoro-phenyl)- | 0.0446 | C26H27FN4O | 430.22 | 431.2 | | P |
| | piperidin-1-ylmethyl]-6- | | | | | | |
| | (6-methoxy-pyridin-3-yl)- | | | | | | |
| | 1-methyl-1H- | | | | | | |
| | benzoimidazole | | | | | | |
| 278 | 2-[4-(2-Fluoro-phenyl)- | 0.0466 | C21H24FN3O | 353.19 | 354.1 | | Q |
| | piperidin-1-ylmethyl]-6- | | | | | | |
| | methoxy-1-methyl-1H- | | | | | | |
| | benzoimidazole | | | | | | |
| 279 | 2-[4-(2-Fluoro-phenyl)- | 0.327 | C21H24FN3 | 337.2 | 338.1 | | R |
| | piperidin-1-ylmethyl]-1,6- | | | | | | |
| | dimethyl-1H- | | | | | | |
| | benzoimidazole | | | | | | |
| 280 | 2-[4-(2-Fluoro-phenyl)- | 0.176 | C26H26FN3 | 399.21 | 400.2 | | P |
| | piperidin-1-ylmethyl]-1- | | | | | | |
| | methyl-5-phenyl-1H- | | | | | | |
| | benzoimidazole | | | | | | |
| 281 | 2-[4-(2-Methoxy-phenyl)- | 1.51 | C21H26N4O | 350.21 | 351.2 | | R1 |
| | piperidin-1-ylmethyl]-1- | | | | | | |
| | methyl-1H- | | | | | | |
| | benzoimidazol-5-ylamine | | | | | | |
| 282 | 2-[4-(2-Methoxy-phenyl)- | 0.0416 | C22H27N3O | 349.22 | 350.2 | | R |
| | piperidin-1-ylmethyl]-1,5- | | | | | | |
| | dimethyl-1H- | | | | | | |
| | benzoimidazole | | | | | | |
| 283 | 2-[4-(2-Methoxy-phenyl)- | 0.0908 | C22H24N4O | 360.2 | 361.1 | | O |
| | piperidin-1-ylmethyl]-1- | | | | | | |
| | methyl-1H- | | | | | | |
| | benzoimidazole-5- | | | | | | |
| | carbonitrile | | | | | | |
| 284 | 5-Fluoro-2-[4-(2- | 0.0394 | C21H24FN3O | 353.19 | 354.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 285 | 5-Chloro-2-[4-(2- | 0.0493 | C21H24ClN3O | 369.16 | 370.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 286 | 2-[4-(2-Fluoro-phenyl)- | 4.62 | C21H24FN3O | 353.19 | 353.1898 | 1.45 | J |
| | piperidin-1-ylmethyl]-1- | | | | | | |
| | methoxymethyl-1H- | | | | | | |
| | benzoimidazole | | | | | | |
| 287 | 1-Methoxymethyl-2-[4- | 1.9 | C22H24F3N3O | 403.19 | 403.1866 | 1.56 | J |
| | (4-trifluoromethyl- | | | | | | |
| | phenyl)-piperidin-1- | | | | | | |
| | ylmethyl]-1H- | | | | | | |
| | benzoimidazole | | | | | | |
| 288 | 2-[4-(2-Methoxy-phenyl)- | 1.68 | C22H25N3O3 | 379.19 | 380.1 | | S1 |
| | piperidin-1-ylmethyl]-1- | | | | | | |
| | methyl-1H- | | | | | | |
| | benzoimidazole-5- | | | | | | |
| | carboxylic acid | | | | | | |
| 289 | 1-(1-Methyl-1H- | >9.88 | C21H23N3O | 333.18 | 334.1 | | N |
| | benzoimidazol-2- | | | | | | |
| | ylmethyl)-5-phenyl- | | | | | | |
| | azepan-2-one | | | | | | |
| 290 | 1-[(1-Methyl-1H- | 5.72 | C22H22F3N3O | 401.17 | 402 | | N |
| | benzoimidazol-2- | | | | | | |
| | yl)methyl]-5-(4- | | | | | | |
| | trifluoromethyl-phenyl)- | | | | | | |
| | azepan-2-one | | | | | | |
| 291 | 5-(2-Methoxy-phenyl)-1- | 4.66 | C22H25N3O2 | 363.19 | 364.1 | | N |
| | (1-methyl-1H- | | | | | | |
| | benzoimidazol-2- | | | | | | |
| | ylmethyl)-azepan-2-one | | | | | | |
| 292 | 2-{[4-(2-Methoxy- | 0.143 | C22H27N3O | 349.22 | 350.2 | | K |
| | phenyl)-3-methyl- | | | | | | |
| | piperidin-1-yl]methyl}-1- | | | | | | |
| | methyl-1H- | | | | | | |
| | benzoimidazole | | | | | | |
| 293 | 5,6-Difluoro-2-{[4-(2- | 0.85 | C22H25F2N3O | 385.2 | 386.1 | | K |
| | methoxy-phenyl)-3- | | | | | | |
| | methyl-piperidin-1- | | | | | | |
| | yl]methyl}-1-methyl-1H- | | | | | | |
| | benzoimidazole | | | | | | |
| 294 | 1-Fluoromethyl-2-[4-(2- | 0.734 | C22H26FN3O | 367.21 | 368.1 | | K |
| | methoxy-phenyl)-3- | | | | | | |
| | methyl-piperidin-1- | | | | | | |
| | ylmethyl]-1H- | | | | | | |
| | benzoimidazole | | | | | | |
| 295 | 2-{2-[4-(2-Fluoro- | 1.74 | C21H24FN3O | 353.19 | 354.1 | | K |
| | phenyl)-piperidin-1- | | | | | | |
| | ylmethyl]-benzoimidazol- | | | | | | |
| | 1-yl}-ethanol | | | | | | |
| 296 | 2-[4-(2-Fluoro-phenyl)- | 0.414 | C22H24FN3O2 | 381.19 | 382.1 | | S |
| | piperidin-1-ylmethyl]-1- | | | | | | |
| | methyl-1H- | | | | | | |
| | benzoimidazole-5- | | | | | | |
| | carboxylic acid methyl | | | | | | |
| | ester | | | | | | |
| 297 | {2-[4-(2-Fluoro-phenyl)- | 0.169 | C21H24FN3O | 353.19 | 354.1 | | U |
| | piperidin-1-ylmethyl]-1- | | | | | | |
| | methyl-1H- | | | | | | |
| | benzoimidazol-5-yl}- | | | | | | |
| | methanol | | | | | | |
| 298 | 2-{2-[4-(2-Fluoro- | >7.34 | C23H28FN3O | 381.22 | 382.1 | | T |
| | phenyl)-piperidin-1- | | | | | | |
| | ylmethyl]-1-methyl-1H- | | | | | | |
| | benzoimidazol-5-yl}- | | | | | | |
| | propan-2-ol | | | | | | |
| 299 | (4aR,10bS)-10-Bromo-7- | 6.12 | C22H23BrClN3 | 443.08 | 446 | | K |
| | chloro-3-(1-methyl-1H- | | | | | | |
| | benzoimidazol-2- | | | | | | |
| | ylmethyl)- | | | | | | |
| | 1,2,3,4,4a,5,6,10b- | | | | | | |
| | octahydro- | | | | | | |
| | benzo[f]isoquinoline | | | | | | |
| 300 | 2-[4-(5-Fluoro-2- | 0.0607 | C21H24FN3O | 353.19 | 354.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 301 | 2-[4-(2-Fluoro-phenyl)- | 0.0423 | C21H24FN3 | 337.2 | 338 | | R |
| | piperidin-1-ylmethyl]-1,7- | | | | | | |
| | dimethyl-1H- | | | | | | |
| | benzoimidazole | | | | | | |
| 302 | 2-[4-(3,5-Dimethyl- | 0.0374 | C22H27N3 | 333.22 | 334.2 | | K |
| | phenyl)-piperidin-1- | | | | | | |
| | ylmethyl]-1-methyl-1H- | | | | | | |
| | benzoimidazole | | | | | | |
| 303 | 1-Methyl-2-[4-(4- | 0.868 | C22H24F3N3 | 387.19 | 388 | | K |
| | trifluoromethyl-phenyl)- | | | | | | |
| | azepan-1-ylmethyl]-1H- | | | | | | |
| | benzoimidazole | | | | | | |
| 304 | 2-[4-(2-Methoxy-phenyl)- | 0.276 | C22H27N3O | 349.22 | 350.2 | | K |
| | azepan-1-ylmethyl]-1- | | | | | | |
| | methyl-1H- | | | | | | |
| | benzoimidazole | | | | | | |
| 305 | 1-Methyl-2-(4-phenyl- | 0.235 | C21H25N3 | 319.2 | 320.2 | | K |
| | azepan-1-ylmethyl)-1H- | | | | | | |
| | benzoimidazole | | | | | | |
| 306 | 2-[4-(2-Fluoro-phenyl)- | 1.59 | C21H24FN3 | 337.2 | 338.1 | | K |
| | azepan-1-ylmethyl]-1- | | | | | | |
| | methyl-1H- | | | | | | |
| | benzoimidazole | | | | | | |
| 307 | 2-[4-(2-Fluoro-phenyl)- | 0.0314 | C21H21FN4 | 348.17 | 349.1 | | O |
| | piperidin-1-ylmethyl]-3- | | | | | | |
| | methyl-3H- | | | | | | |
| | benzoimidazole-4- | | | | | | |
| | carbonitrile | | | | | | |
| 308 | 2-[4-(2-Fluoro-phenyl)- | 0.151 | C26H26FN3 | 399.21 | 400.1 | | P |
| | piperidin-1-ylmethyl]-1- | | | | | | |
| | methyl-7-phenyl-1H- | | | | | | |
| | benzoimidazole | | | | | | |
| 309 | 2-[4-(2-Fluoro-phenyl)- | 0.112 | C26H27FN4O | 430.22 | 431.02 | | P |
| | piperidin-1-ylmethyl]-7- | | | | | | |
| | (6-methoxy-pyridin-3-yl)- | | | | | | |
| | 1-methyl-1H- | | | | | | |
| | benzoimidazole | | | | | | |
| 310 | 2-[4-(4-Fluoro-2-methyl- | 0.0433 | C21H24FN3 | 337.2 | 338.1 | | K |
| | phenyl)-piperidin-1- | | | | | | |
| | ylmethyl]-1-methyl-1H- | | | | | | |
| | benzoimidazole | | | | | | |
| 311 | 2-[4-(2,5-Difluoro- | 0.0161 | C20H21F2N3 | 341.17 | 342.1 | | K |
| | phenyl)-piperidin-1- | | | | | | |
| | ylmethyl]-1-methyl-1H- | | | | | | |
| | benzoimidazole | | | | | | |
| 312 | 7-Bromo-2-[4-(2-fluoro- | 0.0601 | C20H21BrFN3 | 401.09 | 402 | | K |
| | phenyl)-piperidin-1- | | | | | | |
| | ylmethyl]-1-methyl-1H- | | | | | | |
| | benzoimidazole | | | | | | |
| 313 | 1-{2-[1-(1-Methyl-1H- | 0.361 | C22H27N3O | 349.22 | 350.2 | | K |
| | benzoimidazol-2- | | | | | | |
| | ylmethyl)-piperidin-4-yl]- | | | | | | |
| | phenyl}-ethanol | | | | | | |
| 314 | 4-Fluoro-1-methyl-2-[4- | 0.0438 | C21H21F4N3 | 391.17 | 392.2 | | M |
| | (4-trifluoromethyl- | | | | | | |
| | phenyl)-piperidin-1- | | | | | | |
| | ylmethyl]-1H- | | | | | | |
| | benzoimidazole | | | | | | |
| 315 | 2-[4-(3,4-Dimethyl- | 0.308 | C22H27N3 | 333.22 | 334.2 | | K |
| | phenyl)-piperidin-1- | | | | | | |
| | ylmethyl]-1-methyl-1H- | | | | | | |
| | benzoimidazole | | | | | | |
| 316 | 1-Methyl-2-(4-#ml-tolyl- | 0.0515 | C21H25N3 | 319.2 | 320.1 | | K |
| | piperidin-1-ylmethyl)-1H- | | | | | | |
| | benzoimidazole | | | | | | |
| 317 | 1-Methyl-2-[4-(4- | 0.0907 | C22H21F3N4 | 398.17 | 399.1 | | K, M1 |
| | trifluoromethyl-phenyl)- | | | | | | |
| | piperidin-1-ylmethyl]-1H- | | | | | | |
| | benzoimidazole-5- | | | | | | |
| | carbonitrile | | | | | | |
| 318 | 1-Cyclopropyl-2-[4-(2- | 4.69 | C24H26F3N3O | 429.2 | 430.48 | 3.36 | K |
| | methoxy-5- | | | | | | |
| | trifluoromethyl-phenyl)- | | | | | | |
| | piperidin-1-ylmethyl]-1H- | | | | | | |
| | benzoimidazole | | | | | | |
| 319 | 1-(2-Fluoro-ethyl)-2-[4- | 1.65 | C23H25F4N3O | 435.19 | 436.47 | 3.29 | K |
| | (2-methoxy-5- | | | | | | |
| | trifluoromethyl-phenyl)- | | | | | | |
| | piperidin-1-ylmethyl]-1H- | | | | | | |
| | benzoimidazole | | | | | | |
| 320 | 1-Methoxymethyl-2-[4- | 3.8 | C23H26F3N3O2 | 433.2 | 434.46 | 3.31 | K |
| | (2-methoxy-5- | | | | | | |
| | trifluoromethyl-phenyl)- | | | | | | |
| | piperidin-1-ylmethyl]-1H- | | | | | | |
| | benzoimidazole | | | | | | |
| 321 | 5,6-Difluoro-2-[4-(2- | 1.51 | C22H22F5N3O | 439.17 | 440.45 | 3.35 | K |
| | methoxy-5- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 322 | 1-Cyclopropyl-2- | 9.93 | C25H31N3O | 389.25 | 390.56 | 3.34 | K |
| | {[(3R,4R)-4-(2-methoxy- | | | | | | |
| | phenyl)-3,4-dimethyl- | | | | | | |
| | piperidin-1-yl]methyl}- | | | | | | |
| | 1H-benzoimidazole | | | | | | |
| 323 | 1-(2-Methoxy-ethyl)-2-[4- | 0.984 | C24H28F3N3O2 | 447.21 | 448.5 | 3.31 | K |
| | (2-methoxy-5- | | | | | | |
| | trifluoromethyl-phenyl)- | | | | | | |
| | piperidin-1-ylmethyl]-1H- | | | | | | |
| | benzoimidazole | | | | | | |
| 324 | 1-(2-Fluoro-ethyl)-2- | 7.08 | C24H30FN3O | 395.24 | 396.52 | 3.27 | K |
| | {[(3R,4R)-4-(2-methoxy- | | | | | | |
| | phenyl)-3,4-dimethyl- | | | | | | |
| | piperidin-1-yl]methyl}- | | | | | | |
| | 1H-benzoimidazole | | | | | | |
| 325 | 4-Fluoro-2-[4-(2-fluoro- | 0.0338 | C20H21F2N3 | 341.17 | 342.1 | | M |
| | phenyl)-piperidin-1- | | | | | | |
| | ylmethyl]-1-methyl-1H- | | | | | | |
| | benzoimidazole | | | | | | |
| 326 | 2-[4-(2-Fluoro-5- | 0.263 | C23H25F4N3O | 435.19 | 436.21 | 4.35 | J |
| | trifluoromethyl-phenyl)- | | | | | | |
| | piperidin-1-ylmethyl]-1- | | | | | | |
| | (2-methoxy-ethyl)-1H- | | | | | | |
| | benzoimidazole | | | | | | |
| 327 | 1-Cyclopropyl-2-[4-(2- | 1.98 | C23H23F4N3 | 417.18 | 418.24 | 4.41 | J |
| | fluoro-5-trifluoromethyl- | | | | | | |
| | phenyl)-piperidin-1- | | | | | | |
| | ylmethyl]-1H- | | | | | | |
| | benzoimidazole | | | | | | |
| 328 | 5,6-Difluoro-2-[4-(2- | 0.463 | C21H19F6N3 | 427.15 | 428.16 | 4.39 | J |
| | fluoro-5-trifluoromethyl- | | | | | | |
| | phenyl)-piperidin-1- | | | | | | |
| | ylmethyl]-1-methyl-1H- | | | | | | |
| | benzoimidazole | | | | | | |
| 329 | 1-(2-Fluoro-ethyl)-2-[4- | 0.463 | C22H22F5N3 | 423.17 | 424.2 | 4.31 | J |
| | (2-fluoro-5- | | | | | | |
| | trifluoromethyl-phenyl)- | | | | | | |
| | piperidin-1-ylmethyl]-1H- | | | | | | |
| | benzoimidazole | | | | | | |
| 330 | 2-[4-(2-Fluoro-5- | 0.969 | C22H23F4N3O | 421.18 | 422.23 | 4.31 | J |
| | trifluoromethyl-phenyl)- | | | | | | |
| | piperidin-1-ylmethyl]-1- | | | | | | |
| | methoxymethyl-1H- | | | | | | |
| | benzoimidazole | | | | | | |
| 331 | 2-[4-(4-Fluoro-phenyl)- | 0.299 | C21H21FN4 | 348.17 | 349.1 | | K |
| | piperidin-1-ylmethyl]-1- | | | | | | |
| | methyl-1H- | | | | | | |
| | benzoimidazole-5- | | | | | | |
| | carbonitrile | | | | | | |
| 332 | 2-{[4-Methoxy-4-(2- | 5.22 | C23H29N3O2 | 379.23 | 380.26 | 1.77 | J |
| | methoxy-phenyl)-3- | | | | | | |
| | methyl-piperidin-1 | | | | | | |
| | yl]methyl}-1-methyl-1H- | | | | | | |
| | benzoimidazole | | | | | | |
| 333 | 4-Bromo-2-{[4-methoxy- | 4.76 | C23H28BrN3O2 | 457.14 | 460.09 | 1.92 | J |
| | 4-(2-methoxy-phenyl)-3- | | | | | | |
| | methyl-piperidin-1- | | | | | | |
| | yl]methyl}-1-methyl-1H- | | | | | | |
| | benzoimidazole | | | | | | |
| 334 | 1-Fluoromethyl-2-{[4- | >7.08 | C23H28FN3O2 | 397.22 | 398.23 | 1.82 | J |
| | methoxy-4-(2-methoxy- | | | | | | |
| | phenyl)-3-methyl- | | | | | | |
| | piperidin-1-yl]methyl}- | | | | | | |
| | 1H-benzoimidazole | | | | | | |
| 335 | 1-(2-Fluoro-ethyl)-2-{[4- | 10.4 | C24H30FN3O2 | 411.23 | 412.24 | 2.97 | J |
| | methoxy-4-(2-methoxy- | | | | | | |
| | phenyl)-3-methyl- | | | | | | |
| | piperidin-1-yl]methyl}- | | | | | | |
| | 1H-benzoimidazole | | | | | | |
| 336 | 1-(2-Methoxy-ethyl)-2- | 2.36 | C24H31N3O2 | 393.24 | 394.27 | 1.7 | J |
| | [cis-4-(2-methoxy- | | | | | | |
| | phenyl)-3-methyl- | | | | | | |
| | piperidin-1-ylmethyl]-1H- | | | | | | |
| | benzoimidazole | | | | | | |
| 337 | 2-{[Cis-4-(2-Methoxy- | 0.323 | C22H27N3O | 349.22 | 350.27 | 1.73 | J |
| | phenyl)-3-methyl- | | | | | | |
| | piperidin-1-yl]methyl}-1- | | | | | | |
| | methyl-1H- | | | | | | |
| | benzoimidazole | | | | | | |
| 338 | 1-Cyclopropyl-2-{[cis-4- | 1.45 | C24H29N3O | 375.23 | 376.25 | 3.22 | J |
| | (2-methoxy-phenyl)-3- | | | | | | |
| | methyl-piperidin-1- | | | | | | |
| | yl]methyl}-1H- | | | | | | |
| | benzoimidazole | | | | | | |
| 339 | 1-(2-Fluoro-ethyl)-2-{[cis- | 0.604 | C23H28FN3O | 381.22 | 382.27 | 1.73 | J |
| | 4-(2-methoxy-phenyl)-3- | | | | | | |
| | methyl-piperidin-1- | | | | | | |
| | yl]methyl}-1H- | | | | | | |
| | benzoimidazole | | | | | | |
| 340 | 1-Methyl-2-[4-(4- | 0.2 | C21H22F3N3O | 389.17 | 390 | | K |
| | trifluoromethoxy-phenyl)- | | | | | | |
| | piperidin-1-ylmethyl]-1H- | | | | | | |
| | benzoimidazole | | | | | | |
| 341 | 7-Fluoro-1-methyl-2-[4- | 0.633 | C21H21F4N3 | 391.17 | 392 | | K |
| | (4-trifluoromethyl- | | | | | | |
| | phenyl)-piperidin-1- | | | | | | |
| | ylmethyl]-1H- | | | | | | |
| | benzoimidazole | | | | | | |
| 342 | 7-Fluoro-2-[4-(4-fluoro- | 0.0552 | C20H21F2N3 | 341.17 | 342.1 | | K |
| | phenyl)-piperidin-1- | | | | | | |
| | ylmethyl]-1-methyl-1H- | | | | | | |
| | benzoimidazole | | | | | | |
| 343 | 7-Fluoro-2-[4-(2- | 0.0166 | C21H24FN3O | 353.19 | 354.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 344 | 7-Fluoro-2-[4-(2-fluoro- | 0.0206 | C20H21F2N3 | 341.17 | 342.1 | | K |
| | phenyl)-piperidin-1- | | | | | | |
| | ylmethyl]-1-methyl-1H- | | | | | | |
| | benzoimidazole | | | | | | |
| 345 | 5-Chloro-1-methyl-2-[4- | 1.6 | C21H21ClF3N3 | 407.14 | 408.1 | | K |
| | (4-trifluoromethyl- | | | | | | |
| | phenyl)-piperidin-1- | | | | | | |
| | ylmethyl]-1H- | | | | | | |
| | benzoimidazole | | | | | | |
| 346 | 5-Chloro-2-[4-(2-fluoro- | 1.14 | C20H21ClFN3 | 357.14 | 358.1 | | K |
| | phenyl)-piperidin-1- | | | | | | |
| | ylmethyl]-1-methyl-1H- | | | | | | |
| | benzoimidazole | | | | | | |
| 347 | 5-Chloro-2-[4-(2-fluoro- | 0.37 | C20H21ClFN3 | 357.14 | 358.22 | 3.11 | K |
| | phenyl)-piperidin-1- | | | | | | |
| | ylmethyl]-1-methyl-1H- | | | | | | |
| | benzoimidazole | | | | | | |
| 348 | 2-[4-(3,5-Bis- | 0.201 | C22H21F6N3 | 441.16 | 442 | | K |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 349 | 5-Fluoro-2-[4-(2- | 0.0349 | C22H23F4N3O | 421.18 | 422.1 | | K |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 350 | 5-Fluoro-1-methyl-2-[4- | 0.147 | C21H21F4N3 | 391.17 | 392.1 | | K |
| | (4-trifluoromethyl- | | | | | | |
| | phenyl)-piperidin-1- | | | | | | |
| | ylmethyl]-1H- | | | | | | |
| | benzoimidazole | | | | | | |
| 351 | 5-Fluoro-2-[4-(2-fluoro- | 0.0877 | C20H21F2N3 | 341.17 | 342.2 | | K |
| | phenyl)-piperidin-1- | | | | | | |
| | ylmethyl]-1-methyl-1H- | | | | | | |
| | benzoimidazole | | | | | | |
| 352 | 5-Fluoro-2-[4-(2-fluoro- | 0.0885 | C20H21F2N3 | 341.17 | 342.25 | 2.93 | K |
| | phenyl)-piperidin-1- | | | | | | |
| | ylmethyl]-1-methyl-1H- | | | | | | |
| | benzoimidazole | | | | | | |
| 353 | 5-Fluoro-2-[4-(2-fluoro-4- | <0.0402 | C21H20F5N3 | 409.16 | 410.2 | | K |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 354 | 2-[4-(2-Methoxy-4- | <0.0193 | C22H24F3N3O | 403.19 | 404.2 | | K |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 355 | 2-[4-(2-Methoxy-4- | 0.0667 | C22H24F3N3O | 403.19 | 404.21 | 3.19 | K |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 356 | 4-Bromo-2-[4-(2- | 3.9 | C22H23BrF3N3O | 481.1 | 484.07 | 3.47 | J |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 357 | 1-(2-Fluoro-ethyl)-2-[4- | 0.105 | C23H25F4N3O | 435.19 | 436.21 | 3.36 | J |
| | (2-methoxy-4- | | | | | | |
| | trifluoromethyl-phenyl)- | | | | | | |
| | piperidin-1-ylmethyl]-1H- | | | | | | |
| | benzoimidazole | | | | | | |
| 358 | 1-Methoxymethyl-2-[4- | 0.471 | C23H26F3N3O2 | 433.2 | 434.19 | 3.35 | J |
| | (2-methoxy-4- | | | | | | |
| | trifluoromethyl-phenyl)- | | | | | | |
| | piperidin-1-ylmethyl]-1H- | | | | | | |
| | benzoimidazole | | | | | | |
| 359 | 1-Cyclopropyl-2-[4-(2- | 0.94 | C24H26F3N3O | 429.2 | 430.21 | 3.4 | J |
| | methoxy-4- | | | | | | |
| | trifluoromethyl-phenyl)- | | | | | | |
| | piperidin-1-ylmethyl]-1H- | | | | | | |
| | benzoimidazole | | | | | | |
| 360 | 5,6-Difluoro-2-[4-(2- | 0.266 | C22H22F5N3O | 439.17 | 440.17 | 3.41 | J |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 361 | 1-(2-Methoxy-ethyl)-2-[4- | 3.92 | C24H28F3N3O2 | 447.21 | 448.22 | 3.33 | J |
| | (2-methoxy-4- | | | | | | |
| | trifluoromethyl-phenyl)- | | | | | | |
| | piperidin-1-ylmethyl]-1H- | | | | | | |
| | benzoimidazole | | | | | | |
| 362 | 1-Methyl-5- | 5.01 | C22H21F6N3 | 441.16 | 442.1 | | K |
| | trifluoromethyl-2-[4-(4- | | | | | | |
| | trifluoromethyl-phenyl)- | | | | | | |
| | piperidin-1-ylmethyl]-1H- | | | | | | |
| | benzoimidazole | | | | | | |
| 363 | 2-[4-(2-Fluoro-phenyl)- | 2.02 | C21H23FN4O | 366.19 | 368.1 | | V |
| | piperidin-1-ylmethyl]-1- | | | | | | |
| | methyl-1H- | | | | | | |
| | benzoimidazole-5- | | | | | | |
| | carboxylic acid amide | | | | | | |
| 364 | 1-Methyl-2-[4-(4- | 0.498 | C22H23F3N4O | 416.18 | 417.1 | | V |
| | trifluoromethyl-phenyl)- | | | | | | |
| | piperidin-1-ylmethyl]-1H- | | | | | | |
| | benzoimidazole-5- | | | | | | |
| | carboxylic acid amide | | | | | | |
| 365 | 2-[4-(4-Fluoro-phenyl)- | 1.67 | C21H24FN3O | 353.19 | 354.23 | 3.03 | J |
| | piperidin-1-ylmethyl]-1- | | | | | | |
| | methoxymethyl-1H- | | | | | | |
| | benzoimidazole | | | | | | |
| 366 | 4-Bromo-2-[4-(4-fluoro- | 2.14 | C20H21BrFN3 | 401.09 | 402.14 | 3.19 | J |
| | phenyl)-piperidin-1- | | | | | | |
| | ylmethyl]-1-methyl-1H- | | | | | | |
| | benzoimidazole | | | | | | |
| 367 | 2-[4-(4-Fluoro-phenyl)- | 0.905 | C22H26FN3O | 367.21 | 368.31 | 3.03 | J |
| | piperidin-1-ylmethyl]-1- | | | | | | |
| | (2-methoxy-ethyl)-1H- | | | | | | |
| | benzoimidazole | | | | | | |
| 368 | 5,6-Difluoro-2-[4-(4- | 0.475 | C20H20F3N3 | 359.16 | 360.27 | 3.11 | J |
| | fluoro-phenyl)-piperidin- | | | | | | |
| | 1-ylmethyl]-1-methyl-1H- | | | | | | |
| | benzoimidazole | | | | | | |
| 369 | 1-Cyclopropyl-2-[4-(4- | 1.77 | C22H24FN3 | 349.2 | 350.27 | 3.1 | J |
| | fluoro-phenyl)-piperidin- | | | | | | |
| | 1-ylmethyl]-1H- | | | | | | |
| | benzoimidazole | | | | | | |
| 370 | 1-(2-Fluoro-ethyl)-2-[4- | 0.582 | C21H23F2N3 | 355.19 | 356.24 | 3.04 | J |
| | (4-fluoro-phenyl)- | | | | | | |
| | piperidin-1-ylmethyl]-1H- | | | | | | |
| | benzoimidazole | | | | | | |
| 371 | 5-Fluoro-2-[4-(2- | 0.692 | C23H28FN3O | 381.22 | 382.3 | 3.24 | J |
| | isopropoxy-phenyl)- | | | | | | |
| | piperidin-1-ylmethyl]-1- | | | | | | |
| | methyl-1H- | | | | | | |
| | benzoimidazole | | | | | | |
| 372 | 2[4-(5-Chloro-2-fluoro- | 0.308 | C20H20ClF2N3 | 375.13 | 376.22 | 3.11 | J |
| | phenyl)-piperidin-1- | | | | | | |
| | ylmethyl]-5-fluoro-1- | | | | | | |
| | methyl-1H- | | | | | | |
| | benzoimidazole | | | | | | |
| 373 | 5-Chloro-1-methyl-2-[4- | 1.75 | C21H21ClF3N3 | 407.14 | 408.17 | 3.34 | J |
| | (2-trifluoromethyl- | | | | | | |
| | phenyl)-piperidin-1- | | | | | | |
| | ylmethyl]-1H- | | | | | | |
| | benzoimidazole | | | | | | |
| 374 | 5-Fluoro-1-methyl-2-[4- | 0.245 | C21H21F4N3 | 391.17 | 392.23 | 3.21 | J |
| | (3-trifluoromethyl- | | | | | | |
| | phenyl)-piperidin-1- | | | | | | |
| | ylmethyl]-1H- | | | | | | |
| | benzoimidazole | | | | | | |
| 375 | 5-Fluoro-2-[4-(2-fluoro-5- | 0.348 | C21H20F5N3 | 409.16 | 410.22 | 3.22 | J |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 376 | 2-[4-(3,5-Difluoro- | 0.299 | C20H20F3N3 | 359.16 | 360.26 | 3.01 | J |
| | phenyl)-piperidin-1- | | | | | | |
| | ylmethyl]-5-fluoro-1- | | | | | | |
| | methyl-1H- | | | | | | |
| | benzoimidazole | | | | | | |
| 377 | 5-Fluoro-2-[4-(2- | 0.853 | C22H23F4N3O | 421.18 | 422.22 | 3.29 | J |
| | methoxy-5- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 378 | 5-Fluoro-2-[4-(3- | 0.568 | C21H24FN3O | 353.19 | 354.26 | 2.91 | J |
| | methoxy-phenyl)- | | | | | | |
| | piperidin-1-ylmethyl]-1- | | | | | | |
| | methyl-1H- | | | | | | |
| | benzoimidazole | | | | | | |
| 379 | 5-Chloro-2-[4-(2- | 1.57 | C23H28ClN3O | 397.19 | 398.26 | 3.4 | J |
| | isopropoxy-phenyl)- | | | | | | |
| | piperidin-1-ylmethyl]-1- | | | | | | |
| | methyl-1H- | | | | | | |
| | benzoimidazole | | | | | | |
| 380 | 5-Chloro-2-[4-(5-chloro- | 1.73 | C20H20Cl2FN3 | 391.1 | 392.14 | 3.27 | J |
| | 2-fluoro-phenyl)- | | | | | | |
| | piperidin-1-ylmethyl]-1- | | | | | | |
| | methyl-1H- | | | | | | |
| | benzoimidazole | | | | | | |
| 381 | 5-Chloro-1-methyl-2-[4- | 1.59 | C21H21ClF3N3 | 407.14 | 408.17 | 3.37 | J |
| | (3-trifluoromethyl- | | | | | | |
| | phenyl)-piperidin-1- | | | | | | |
| | ylmethyl]-1H- | | | | | | |
| | benzoimidazole | | | | | | |
| 382 | 5-Chloro-2-[4-(3,5- | 7.06 | C20H20ClF2N3 | 375.13 | 376.17 | 3.18 | J |
| | difluoro-phenyl)- | | | | | | |
| | piperidin-1-ylmethyl]-1- | | | | | | |
| | methyl-1H- | | | | | | |
| | benzoimidazole | | | | | | |
| 383 | 5-Chloro-2-[4-(2- | 0.307 | C22H23ClF3N3O | 437.15 | 438.19 | 3.47 | J |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 384 | 5-Chloro-2-[4-(2-fluoro-4- | 0.493 | C21H20ClF4N3 | 425.13 | 426.17 | 3.42 | J |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 385 | 5-Chloro-2-[4-(3- | 2.76 | C21H24ClN3O | 369.16 | 370.23 | 3.1 | J |
| | methoxy-phenyl)- | | | | | | |
| | piperidin-1-ylmethyl]-1- | | | | | | |
| | methyl-1H- | | | | | | |
| | benzoimidazole | | | | | | |
| 386 | 5-Chloro-2-[4-(2- | >3.74 | C22H23ClF3N3O | 437.15 | 437.15 | 3.44 | J |
| | methoxy-5- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 387 | 2-[4-(5-Chloro-2-fluoro- | 0.201 | C21H20ClFN4 | 382.14 | 383.16 | 3.06 | J |
| | phenyl)-piperidin-1- | | | | | | |
| | ylmethyl]-1-methyl-1H- | | | | | | |
| | benzoimidazole-5- | | | | | | |
| | carbonitrile | | | | | | |
| 388 | 2-[4-(2-methoxy-5- | 0.583 | C23H23F3N4O | 428.18 | 429.21 | 3.24 | J |
| | trifluoromethyl-phenyl)- | | | | | | |
| | piperidin-1-ylmethyl]-1- | | | | | | |
| | methyl-1H- | | | | | | |
| | benzoimidazole-5- | | | | | | |
| | carbonitrile | | | | | | |
| 389 | 1-Methyl-2-[4-(3- | 0.285 | C22H21F3N4 | 398.17 | 399.2 | 3.17 | J |
| | trifluoromethyl-phenyl)- | | | | | | |
| | piperidin-1-ylmethyl]-1H- | | | | | | |
| | benzoimidazole-5- | | | | | | |
| | carbonitrile | | | | | | |
| 390 | 2-[4-(2-Methoxy-4- | 0.052 | C23H23F3N4O | 428.18 | 429.2 | 3.28 | J |
| | trifluoromethyl-phenyl)- | | | | | | |
| | piperidin-1-ylmethyl]-1- | | | | | | |
| | methyl-1H- | | | | | | |
| | benzoimidazole-5- | | | | | | |
| | carbonitrile | | | | | | |
| 391 | 2-[4-(2-Fluoro-4- | 0.0444 | C22H20F4N4 | 416.16 | 417.18 | 3.23 | J |
| | trifluoromethyl-phenyl)- | | | | | | |
| | piperidin-1-ylmethyl]-1- | | | | | | |
| | methyl-1H- | | | | | | |
| | benzoimidazole-5- | | | | | | |
| | carbonitrile | | | | | | |
| 392 | 2-[4-(2-Fluoro-5- | 0.441 | C22H20F4N4 | 416.16 | 417.2 | 3.17 | J |
| | trifluoromethyl-phenyl)- | | | | | | |
| | piperidin-1-ylmethyl]-1- | | | | | | |
| | methyl-1H- | | | | | | |
| | benzoimidazole-5- | | | | | | |
| | carbonitrile | | | | | | |
| 393 | 4-Fluoro-2-[4-(2- | 1.03 | C23H28FN3O | 381.22 | 382.36 | 3 | L |
| | isopropoxy-phenyl)- | | | | | | |
| | piperidin-1-ylmethyl]-1- | | | | | | |
| | methyl-1H- | | | | | | |
| | benzoimidazole | | | | | | |
| 394 | 4-Fluoro-1-methyl-2-[4- | 0.232 | C21H21F4N3 | 391.17 | 392.27 | 2.92 | L |
| | (2-trifluoromethyl- | | | | | | |
| | phenyl)-piperidin-1- | | | | | | |
| | ylmethyl]-1H- | | | | | | |
| | benzoimidazole | | | | | | |
| 395 | 2-[4-(3,5-Difluoro- | 0.328 | C20H20F3N3 | 359.16 | 360.28 | 2.84 | L |
| | phenyl)-piperidin-1- | | | | | | |
| | ylmethyl]-4-fluoro-1- | | | | | | |
| | methyl-1H- | | | | | | |
| | benzoimidazole | | | | | | |
| 396 | 4-Fluoro-1-methyl-2-[4- | 0.123 | C21H21F4N3 | 391.17 | 392.27 | 2.99 | L |
| | (3-trifluoromethyl- | | | | | | |
| | phenyl)-piperidin-1- | | | | | | |
| | ylmethyl]-1H- | | | | | | |
| | benzoimidazole | | | | | | |
| 397 | 4-Fluoro-2-[4-(2- | 0.435 | C22H23F4N3O | 421.18 | 422.28 | 3.02 | L |
| | methoxy-5- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 398 | 4-Fluoro-2-[4-(2-fluoro-4- | 0.0402 | C21H20F5N3 | 409.16 | 410.27 | 3.02 | L |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 399 | 4-Fluoro-2-[4-(2- | <0.0124 | C22H23F4N3O | 421.18 | 422.4 | | L |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 400 | 4-Fluoro-2-[4-(2- | 0.0436 | C22H23F4N3O | 421.18 | 422.28 | 3.06 | L |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 401 | 4-Fluoro-2-[4-(2-fluoro-5- | 0.161 | C21H20F5N3 | 409.16 | 410.28 | 3 | L |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 402 | 2-[4-(2-Isopropoxy- | 0.523 | C24H28N4O | 388.23 | 389.24 | 3.2 | L |
| | phenyl)-piperidin-1- | | | | | | |
| | ylmethyl]-1-methyl-1H- | | | | | | |
| | benzoimidazole-5- | | | | | | |
| | carbonitrile | | | | | | |
| 403 | 1-Methyl-2-[4-(2- | 0.224 | C22H21F3N4 | 398.17 | 399.2 | 3.12 | L |
| | trifluoromethyl-phenyl)- | | | | | | |
| | piperidin-1-ylmethyl]-1H- | | | | | | |
| | benzoimidazole-5- | | | | | | |
| | carbonitrile | | | | | | |
| 404 | 1-Methyl-2-{[Cis-3- | 0.471 | C22H24F3N3 | 387.19 | 388.2 | | K |
| | methyl-4-(4- | | | | | | |
| | trifluoromethyl-phenyl)- | | | | | | |
| | piperidin-1-yl]methyl}- | | | | | | |
| | 1H-benzoimidazole | | | | | | |
| 405 | 2-{[Cis-4-(2-Fluoro- | 0.124 | C21H24FN3 | 337.2 | 338.2 | | K |
| | phenyl)-3-methyl- | | | | | | |
| | piperidin-1-yl]methyl}-1- | | | | | | |
| | methyl-1H- | | | | | | |
| | benzoimidazole | | | | | | |
| 406 | 7-Fluoro-2-[4-(2- | 0.117 | C22H23F4N3O | 421.18 | 422.2 | | K |
| | methoxy-4- | | | | | | |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 407 | 7-Fluoro-2-[4-(4-fluoro-2- | 0.167 | C21H23F2N3O | 371.18 | 372.2 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 408 | 5-Fluoro-2-[4-(4-fluoro-2- | 0.0672 | C21H23F2N3O | 371.18 | 372.2 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 409 | 2-[4-(4-Fluoro-2- | 0.0634 | C21H24FN3O | 353.19 | 354.4 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 410 | 2-{[(3S,4R)-3- | 5.26 | C28H28FN3O3 | 473.21 | 474.2 | | K |
| | (Benzo[1,3]dioxol-5- | | | | | | |
| | yloxymethyl)-4-(4-fluoro- | | | | | | |
| | phenyl)-piperidin-1- | | | | | | |
| | yl]methyl}-1-methyl-1H- | | | | | | |
| | benzoimidazole | | | | | | |
| 411 | 2-{[3,3-Dimethyl-4-(4- | 0.633 | C23H26F3N3 | 401.21 | 402.2 | | K |
| | trifluoromethyl-phenyl)- | | | | | | |
| | piperidin-1-yl]methyl}-1- | | | | | | |
| | methyl-1H- | | | | | | |
| | benzoimidazole | | | | | | |
| 412 | 2-[4-(4-Isopropoxy- | 0.105 | C23H29N3O | 363.23 | 364.1 | | K |
| | phenyl)-piperidin-1- | | | | | | |
| | ylmethyl]-1-methyl-1H- | | | | | | |
| | benzoimidazole | | | | | | |
| 413 | 1-Methyl-2-[4-(2- | 0.0776 | C21H22F3N3O | 389.17 | 390.1 | | K |
| | trifluoromethoxy-phenyl)- | | | | | | |
| | piperidin-1-ylmethyl]-1H- | | | | | | |
| | benzoimidazole | | | | | | |
| 414 | 2-[4-(3,5-Difluoro- | 0.126 | C20H21F2N3 | 341.17 | 342.1 | | K |
| | phenyl)-piperidin-1- | | | | | | |
| | ylmethyl]-1-methyl-1H- | | | | | | |
| | benzoimidazole | | | | | | |
| 415 | 2-[4-(3,5-Difluoro- | 0.189 | C20H21F2N3 | 341.17 | 342.3 | | K |
| | phenyl)-piperidin-1- | | | | | | |
| | ylmethyl]-1-methyl-1H- | | | | | | |
| | benzoimidazole | | | | | | |
| 416 | 2-[1-(1-Methyl-1H- | 0.168 | C21H22N4 | 330.18 | 331.3 | | K |
| | benzoimidazol-2- | | | | | | |
| | ylmethyl)-piperidin-4-yl]- | | | | | | |
| | benzonitrile | | | | | | |
| 417 | 2-[4-(3-Chloro-phenyl)- | 0.18 | C20H22ClN3 | 339.15 | 340.2 | | K |
| | piperidin-1-ylmethyl]-1- | | | | | | |
| | methyl-1H- | | | | | | |
| | benzoimidazole | | | | | | |
| 418 | 3-[1-(1-Methyl-1H- | 1.61 | C21H22N4 | 330.18 | 331.3 | | K |
| | benzoimidazol-2- | | | | | | |
| | ylmethyl)-piperidin-4-yl]- | | | | | | |
| | benzonitrile | | | | | | |
| 419 | 2-[4-(3-tert-Butyl- | 0.149 | C24H31N3 | 361.25 | 362.3 | | K |
| | phenyl)-piperidin-1- | | | | | | |
| | ylmethyl]-1-methyl-1H- | | | | | | |
| | benzoimidazole | | | | | | |
| 420 | 2-[4-(4-Chloro-phenyl)- | 0.664 | C20H22ClN3 | 339.15 | 341.2 | | K |
| | piperidin-1-ylmethyl]-1- | | | | | | |
| | methyl-1H- | | | | | | |
| | benzoimidazole | | | | | | |
| 421 | 2-[4-(4-Methoxy-phenyl)- | 0.213 | C21H25N3O | 335.2 | 336.3 | | K |
| | piperidin-1-ylmethyl]-1- | | | | | | |
| | methyl-1H- | | | | | | |
| | benzoimidazole | | | | | | |
| 422 | 2-[4-(2,4-Difluoro- | 0.0146 | C20H21F2N3 | 341.17 | 342.2 | | K |
| | phenyl)-piperidin-1- | | | | | | |
| | ylmethyl]-1-methyl-1H- | | | | | | |
| | benzoimidazole | | | | | | |
| 423 | 2-[4-(2,4-Difluoro- | 0.0352 | C20H20F3N3 | 359.16 | 360.2 | | K |
| | phenyl)-piperidin-1- | | | | | | |
| | ylmethyl]-5-fluoro-1- | | | | | | |
| | methyl-1H- | | | | | | |
| | benzoimidazole | | | | | | |
| 424 | 2-[4-(2,4-Difluoro- | 0.0483 | C20H20F3N3 | 359.16 | 360.2 | | K |
| | phenyl)-piperidin-1- | | | | | | |
| | ylmethyl]-7-fluoro-1- | | | | | | |
| | methyl-1H- | | | | | | |
| | benzoimidazole | | | | | | |
| 425 | 2-[4-(4-Fluoro-3- | 0.434 | C21H24FN3O | 353.19 | 354.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 426 | 2-[4-(4-Ethyl-phenyl)- | 0.0892 | C22H27N3 | 333.22 | 334.1 | | K |
| | piperidin-1-ylmethyl]-1- | | | | | | |
| | methyl-1H- | | | | | | |
| | benzoimidazole | | | | | | |
| 427 | 2-[4-(2,4-Difluoro- | 0.14 | C21H20F2N4 | 366.17 | 367.2 | | K |
| | phenyl)-piperidin-1- | | | | | | |
| | ylmethyl]-1-methyl-1H- | | | | | | |
| | benzoimidazole-5- | | | | | | |
| | carbonitrile | | | | | | |
| 428 | 1-(1-Methyl-1H- | 1.12 | C21H22F3N3O | 389.17 | 390.2 | | K |
| | benzoimidazol-2- | | | | | | |
| | ylmethyl)-4-(4- | | | | | | |
| | trifluoromethyl-phenyl)- | | | | | | |
| | piperidin-3-ol | | | | | | |
| 429 | 1-Methyl-2-(4-o-tolyl- | 0.0651 | C21H25N3 | 319.2 | 320.2 | | K |
| | piperidin-1-ylmethyl)-1H- | | | | | | |
| | benzoimidazole | | | | | | |
| 430 | 2-[4-(3-Fluoro-phenyl)- | 3.31 | C20H22FN3 | 323.18 | 324.2 | | K |
| | piperidin-1-ylmethyl]-1- | | | | | | |
| | methyl-1H- | | | | | | |
| | benzoimidazole | | | | | | |
| 431 | 2-[4-(6-Fluoro-chroman- | 5.34 | C23H26FN3O | 379.21 | 381.2 | | K |
| | 8-yl)-piperidin-1- | | | | | | |
| | ylmethyl]-1-methyl-1H- | | | | | | |
| | benzoimidazole | | | | | | |
| 432 | 2-{[4-(4-Chloro-phenyl)- | 0.257 | C20H21ClFN3 | 357.14 | 358.1 | | K |
| | 4-fluoro-piperidin-1- | | | | | | |
| | yl]methyl}-1-methyl-1H- | | | | | | |
| | benzoimidazole | | | | | | |
| 433 | 2-{[4-Fluoro-4-(4- | 0.122 | C21H21F4N3 | 391.17 | 392.1 | | K |
| | trifluoromethyl-phenyl)- | | | | | | |
| | piperidin-1-yl]methyl}-1- | | | | | | |
| | methyl-1H- | | | | | | |
| | benzoimidazole | | | | | | |
| 434 | 2-{[4-Fluoro-4-(4-fluoro- | 0.308 | C20H21F2N3 | 341.17 | 342.1 | | K |
| | phenyl)-piperidin-1- | | | | | | |
| | yl]methyl}-1-methyl-1H- | | | | | | |
| | benzoimidazole | | | | | | |
| 435 | 3-[1-(1-Methyl-1H- | 3.3 | C27H29N5O | 439.24 | 440.4 | | K |
| | benzoimidazol-2- | | | | | | |
| | ylmethyl)-piperidin-4-yl]- | | | | | | |
| | N-(6-methyl-pyridin-2-yl)- | | | | | | |
| | benzamide | | | | | | |
| 436 | N-Cyclopropylmethyl-3- | 2.21 | C26H36N4O | 444.29 | 445.4 | | K |
| | [1-(1-methyl-1H- | | | | | | |
| | benzoimidazol-2- | | | | | | |
| | ylmethyl)-piperidin-4-yl]- | | | | | | |
| | N-propyl-benzamide | | | | | | |
| 437 | N-Cyclopentyl-4-[1-(1- | 5.27 | C26H32N4O | 416.26 | 417.4 | | K |
| | methyl-1H- | | | | | | |
| | benzoimidazol-2- | | | | | | |
| | ylmethyl)-piperidin-4-yl]- | | | | | | |
| | benzamide | | | | | | |
| 438 | 2-[4-(2-Fluoro-6- | 0.081 | C21H24FN3O | 353.19 | 354.1 | | K |
| | methoxy-phenyl)- | | | | | | |
| | piperidin-1-ylmethyl]-1- | | | | | | |
| | methyl-1H- | | | | | | |
| | benzoimidazole | | | | | | |
| 439 | 7-Fluoro-2-{[4-fluoro-4- | 0.46 | C20H20F3N3 | 359.16 | 360.2 | | K |
| | (4-fluoro-phenyl)- | | | | | | |
| | piperidin-1-yl]methyl}-1- | | | | | | |
| | methyl-1H- | | | | | | |
| | benzoimidazole | | | | | | |
| 440 | 2-[(3,5-Dimethyl-4- | 2.85 | C22H25N3 | 331.2 | 332.4 | | K |
| | phenyl-3,6-dihydro-2H- | | | | | | |
| | pyridin-1-yl)methyl]-1- | | | | | | |
| | methyl-1H- | | | | | | |
| | benzoimidazole | | | | | | |
| 441 | 2-{[3-Fluoro-4-(4- | 0.157 | C21H21F4N3 | 391.17 | 392.2 | | K |
| | trifluoromethyl-phenyl)- | | | | | | |
| | piperidin-1-yl]methyl}-1- | | | | | | |
| | methyl-1H- | | | | | | |
| | benzoimidazole | | | | | | |
| 442 | 2-[4-(4-Ethoxy-phenyl)- | 0.0807 | C22H27N3O | 349.22 | 350.2 | | K |
| | piperidin-1-ylmethyl]-1- | | | | | | |
| | methyl-1H- | | | | | | |
| | benzoimidazole | | | | | | |
| 443 | 2-[4-(3-Chloro-4-fluoro- | 0.057 | C20H21ClFN3 | 357.14 | 358.3 | | K |
| | phenyl)-piperidin-1- | | | | | | |
| | ylmethyl]-1-methyl-1H- | | | | | | |
| | benzoimidazole | | | | | | |
| 444 | 2-[4-(3-Chloro-4-fluoro- | 0.131 | C20H20ClF2N3 | 375.13 | 376.2 | | K |
| | phenyl)-piperidin-1- | | | | | | |
| | ylmethyl]-7-fluoro-1- | | | | | | |
| | methyl-1H- | | | | | | |
| | benzoimidazole | | | | | | |
| 445 | 7-Fluoro-2-{[3-fluoro-4- | 0.137 | C21H20F5N3 | 409.16 | 410.2 | | K |
| | (4-trifluoromethyl- | | | | | | |
| | phenyl)-piperidin-1- | | | | | | |
| | yl]methyl}-1-methyl-1H- | | | | | | |
| | benzoimidazole | | | | | | |
| 446 | 2-[4-(4-Chloro-3- | 0.0429 | C21H21ClF3N3 | 407.14 | 408.1 | 2.2 | K |
| | trifluoromethyl-phenyl)- | | | | | | |
| | piperidin-1-ylmethyl]-1- | | | | | | |
| | methyl-1H- | | | | | | |
| | benzoimidazole | | | | | | |
| 447 | 2-[4-(3-Chloro-4-fluoro- | 0.0456 | C20H20ClF2N3 | 375.13 | 376.2 | | K |
| | phenyl)-piperidin-1- | | | | | | |
| | ylmethyl]-5-fluoro-1- | | | | | | |
| | methyl-1H- | | | | | | |
| | benzoimidazole | | | | | | |
| 448 | 1-Methyl-2-(4-#pl-tolyl- | 0.0562 | C21H25N3 | 319.2 | 320.3 | 1.9 | K |
| | piperidin-1-ylmethyl)-1H- | | | | | | |
| | benzoimidazole | | | | | | |
| 449 | 2-[(4-Fluoro-4-#ol-tolyl- | 0.252 | C21H24FN3 | 337.2 | 338.1 | | K |
| | piperidin-1-yl)methyl]-1- | | | | | | |
| | methyl-1H- | | | | | | |
| | benzoimidazole | | | | | | |
| 450 | 2-[4-(4-Chloro-3-fluoro- | 0.079 | C20H21ClFN3 | 357.14 | 358.1 | 2 | K |
| | phenyl)-piperidin-1- | | | | | | |
| | ylmethyl]-1-methyl-1H- | | | | | | |
| | benzoimidazole | | | | | | |
| 451 | 2-[4-(4-Chloro-2-fluoro- | 0.00757 | C20H21ClFN3 | 357.14 | 358.2 | 1.7 | K |
| | phenyl)-piperidin-1- | | | | | | |
| | ylmethyl]-1-methyl-1H- | | | | | | |
| | benzoimidazole | | | | | | |
| 452 | 2-[4-(2,3-Difluoro- | 0.0202 | C20H21F2N3 | 341.17 | 342.4 | 1.9 | K |
| | phenyl)-piperidin-1- | | | | | | |
| | ylmethyl]-1-methyl-1H- | | | | | | |
| | benzoimidazole | | | | | | |
| 453 | 2-[4-(4-Chloro-3-fluoro- | 0.157 | C20H20ClF2N3 | 375.13 | 376.1 | 1.64 | K |
| | phenyl)-piperidin-1- | | | | | | |
| | ylmethyl]-7-fluoro-1- | | | | | | |
| | methyl-1H- | | | | | | |
| | benzoimidazole | | | | | | |
| 454 | 2-[4-(4-Chloro-3-fluoro- | 0.0882 | C20H20ClF2N3 | 375.13 | 376.1 | 1.72 | K |
| | phenyl)-piperidin-1- | | | | | | |
| | ylmethyl]-5-fluoro-1- | | | | | | |
| | methyl-1H- | | | | | | |
| | benzoimidazole | | | | | | |
| 455 | 2-(4-{4-[((2R,6S)-2,6- | 2.59 | C27H36N4O | 432.29 | 433.4 | | K |
| | Dimethyl-morpholin-4- | | | | | | |
| | yl)methyl]-phenyl}- | | | | | | |
| | piperidin-1-ylmethyl)-1- | | | | | | |
| | methyl-1H- | | | | | | |
| | benzoimidazole | | | | | | |
| 456 | 2-{[Cis-4-(3-Chloro-4- | 0.181 | C21H23ClFN3 | 371.16 | 372.1 | 1.85 | K |
| | fluoro-phenyl)-3 methyl- | | | | | | |
| | piperidin-1-yl]methyl}-1- | | | | | | |
| | methyl-1H- | | | | | | |
| | benzoimidazole | | | | | | |
| 457 | 1-Methyl-2-{[Cis-2- | 0.454 | C22H24F3N3 | 387.19 | 388.2 | | K |
| | methyl-4-(4- | | | | | | |
| | trifluoromethyl-phenyl)- | | | | | | |
| | piperidin-1-yl]methyl}- | | | | | | |
| | 1H-benzoimidazole | | | | | | |
| 458 | 2-[4-(4-Chloro-2-fluoro- | 0.0419 | C20H20ClF2N3 | 375.13 | 376.3 | | K |
| | phenyl)-piperidin-1- | | | | | | |
| | ylmethyl]-5-fluoro-1- | | | | | | |
| | methyl-1H- | | | | | | |
| | benzoimidazole | | | | | | |
| 459 | 2-{[Cis-4-(2-Methoxy-4- | 0.128 | C23H26F3N3O | 417.2 | 418.1 | | K |
| | trifluoromethyl-phenyl)-3- | | | | | | |
| | methyl-piperidin-1- | | | | | | |
| | yl]methyl}-1-methyl-1H- | | | | | | |
| | benzoimidazole | | | | | | |
| 460 | 2-[(6-(4-Fluoro-phenyl)- | 0.106 | C21H22FN3 | 335.18 | 336.1 | 2.03 | K |
| | 3-aza-bicyclo[4,1.0]hept- | | | | | | |
| | 3-ylmethyl]-1-methyl-1H- | | | | | | |
| | benzoimidazole | | | | | | |
| 461 | 2-{[Cis-4-(4-Chloro-2- | 0.0303 | C21H23ClFN3 | 371.16 | 372.1 | 2.3 | K |
| | fluoro-phenyl)-3-methyl- | | | | | | |
| | piperidin-1-yl]methyl}-1- | | | | | | |
| | methyl-1H- | | | | | | |
| | benzoimidazole | | | | | | |
| 462 | 2-{[Cis-4-(3,4-Difluoro- | 0.241 | C21H23F2N3 | 355.19 | 356.1 | | K |
| | phenyl)-3-methyl- | | | | | | |
| | piperidin-1-yl]methyl}-1- | | | | | | |
| | methyl-1H- | | | | | | |
| | benzoimidazole | | | | | | |
| 463 | 2-{[Cis-4-(2,4-Difluoro- | 0.0407 | C21H23F2N3 | 355.19 | 256.1 | | K |
| | phenyl)-3-methyl- | | | | | | |
| | piperidin-1-yl]methyl}-1- | | | | | | |
| | methyl-1H- | | | | | | |
| | benzoimidazole | | | | | | |
| 464 | 2-{[Cis-4-(4-Fluoro-2- | 0.125 | C22H26FN3O | 367.21 | 368.2 | | K |
| | methoxy-phenyl)-3- | | | | | | |
| | methyl-piperidin-1- | | | | | | |
| | yl]methyl}-1-methyl-1H- | | | | | | |
| | benzoimidazole | | | | | | |
| 465 | 2-({Cis-4-[3-fluoro-4- | 0.375 | C22H23F4N3 | 405.18 | 406.3 | 2.1 | K |
| | (trifluoromethyl)phenyl]- | | | | | | |
| | 3-methylpiperidin-1- | | | | | | |
| | yl}methyl)-1-methyl-1H- | | | | | | |
| | benzimidazole | | | | | | |
| 466 | 2-({Cis-4-[2,4- | 0.161 | C23H23F6N3 | 455.18 | 456.3 | 2.4 | K |
| | bis(trifluoromethyl)phenyl]- | | | | | | |
| | 3-methylpiperidin-1- | | | | | | |
| | yl}methyl)-1-methyl-1H- | | | | | | |
| | benzimidazole | | | | | | |
| 467 | 2-({Cis-4-[2-chloro-4- | 0.0531 | C22H23ClF3N3 | 421.15 | 422.4 | 2.2 | K |
| | (trifluoromethyl)phenyl]- | | | | | | |
| | 3-methylpiperidin-1- | | | | | | |
| | yl}methyl)-1-methyl-1H- | | | | | | |
| | benzimidazole | | | | | | |
| 468 | 2-{[Cis-4-(2,4- | 0.0862 | C21H23Cl2N3 | 387.13 | 388.1 | 2.3 | K |
| | dichlorophenyl)-3- | | | | | | |
| | methylpiperidin-1- | | | | | | |
| | yl]methyl}-1-methyl-1H- | | | | | | |
| | benzimidazole | | | | | | |
| 469 | 2-{[Cis-4-(4-chloro-2- | >0.351 | C22H26ClN3 | 367.18 | 368.1 | 2.2 | K |
| | methylphenyl)-3- | | | | | | |
| | methylpiperidin-1- | | | | | | |
| | yl]methyl}-1-methyl-1H- | | | | | | |
| | benzimidazole | | | | | | |
| 470 | (4aR,10bS)-3-[(1-methyl- | 1.52 | C21H23N3O | 333.43 | 334 | | K |
| | 1H-benzimidazol-2- | | | | | | |
| | yl)methyl]- | | | | | | |
| | 1,3,4,4a,5,10b- | | | | | | |
| | hexahydro-2H- | | | | | | |
| | chromeno[3,4-c]pyridine | | | | | | |
| 471 | (4aR,10bR)-3-[(1-methyl- | 2.07 | C21H23N3O | 333.43 | 334 | | K |
| | 1H-benzimidazol-2- | | | | | | |
| | yl)methyl]- | | | | | | |
| | 1,3,4,4a,5,10b- | | | | | | |
| | hexahydro-2H- | | | | | | |
| | chromeno[3,4-c]pyridine | | | | | | |
| 472 | 2-{[Cis-4-(3,4- | 0.152 | C21H23F2N3 | 355.19 | 356.2 | 6.12* | K1 |
| | difluorophenyl)-3- | | | | | | |
| | methylpiperidin-1- | | | | | | |
| | yl]methyl}-1-methyl-1H- | | | | | | |
| | benzimidazole, | | | | | | |
| | Enantiomer 1 | | | | | | |
| 473 | 2-{[Cis-4-(3,4- | 1.03 | C21H23F2N3 | 355.19 | 356.2 | 9.82* | K1 |
| | difluorophenyl)-3- | | | | | | |
| | methylpiperidin-1- | | | | | | |
| | yl]methyl}-1-methyl-1H- | | | | | | |
| | benzimidazole, | | | | | | |
| | Enantiomer 2 | | | | | | |
| 474 | 2-({4-[2-fluoro-4- | 0.0767 | C22H23F4N3O | 421.18 | 422.3 | 2.3 | K |
| | (trifluoromethoxy)phenyl]- | | | | | | |
| | 3-methylpiperidin-1- | | | | | | |
| | yl}methyl)-1-methyl-1H- | | | | | | |
| | benzimidazole | | | | | | |

O. Biological Protocols

In Vitro Assays

Procedure for mGluR2 Potentiator Screen NLB Methods EC10-EC20 Challenge Cell Culture and Plating:

Cells used for this screen are HEK cells stably transfected with the mGluR2 receptor (metabotropic glutamate receptor 2) and the Gα15 G protein. Clones were identified by functional activity (FLIPR). Cells are grown in growth media containing: DMEM High Glucose with Glutamine and Na Pyruvate (GIBCO), 10% (v/v) Heat inactivate FBS (GIBCO) G418 500 ug/ml (from 50 mg/ml stock) (GIBCO) and Blasticidin 3 ug/ml (from 5 mg/ml stock made in H2O) (Invitrogen).

2 days before the assay cell are trypsinized with 0.25% trysin/EDTA (GIBCO), spun down at 1000 rpm for 5 minutes, resuspended in growth media and plated on polystyrene 384 well black wall/clear bottom poly-D-lysine coated plates at a density of approximately 18,000 cells/well in a volume of 50 μL per well. One day before the assay the growth media is removed from the plates by flicking and replaced with media containing DMEM High Glucose without Glutamine and Na Pyruvate (GIBCO) and 10% (v/v) dialyzed FBS (GIBCO). The reason for the removal of glutamine the day before the assay is to minimize the amount of glutamate that will be present during the assay, as endogenous glutarnate released from the cells can reduce the fluorescent response and interfere with the FLIPR screen.

FLIPR Methods and Data Analysis:

On the day of the assay, the FLIPR assay is performed using the following methods:

Assay buffer:

| Compound | g/L | MW | [concentration] |
|---|---|---|---|
| NaCl | 8.47 | 58.44 | 145 mM |
| Glucose | 1.8 | 180.2 | 10 mM |
| KCl | .37 | 74.56 | 5 mM |
| MgSO₄ | 1 ml 1M Stock | 246.48 | 1 mM |
| HEPES | 2.38 | 238.3 | 10 mM |
| CaCl₂ | 2 ml 1M Stock | 110.99 | 2 mM |

The pH is adjusted to 7.4 with 1M NaOH. Prepare a 2 mM (approx.) stock solution of Fluo-4.am (Molecular Probes) dye in DMSO −22 μl DMSO per 50 ug vial (440 μL per 1 mg vial). Make a 1 mM (approx.) flou-4, PA working solution per vial by adding 22 μl of 20% pluronic acid (PA) (Molecular Probes) in DMSO to each 50 ug vial (440 μL per 1 mg vial). Prepare a 250 mM Probenecid (Sigma) stock solution by dissolving 0.71 g into 5 ml 1N NaOH and 5 ml assay buffer (for each liter of assay wash buffer). Make 4 uM (approx.) dye incubation media by adding 2.50 ug vials per 11 ml DMEM high glucose without glutamine (220 ml per 1 mg vial). Add 110 μL probenecid stock per 11 ml (2.5 mM final [concentration]). To the dye media add 3 units/ml of glutamic-pyruvic transaminase (GPT, Sigma) and 3 mM Na Pyruvate. The assay has worked with dye concentrations from 2 uM to 8 uM dye as well. To the assay buffer from drug preparation, add 1.83 mls DMSO and 400 μL. 15.8% P104 (from New Leads biology) per liter for final concentrations of 0.18% DMSO and 0.006% P104. To the assay buffer for cell washing, add probenecid in the same manner and concentration that was used for the dye media.

Remove growth media from cell plates by flicking. Add 50 μl/well dye solution. Incubate 1 hour at 37° C. and 5% CO₂. Remove dye solution and wash 3 times with assay buffer+probenecid (100 μl probenecid stock per 10 ml buffer), leaving 30 μL/well assay buffer. Wait at least 10-15 minutes. Compounds and agonist challenge additions are performed with the FLIPR. The in addition is for test compounds, which are added as 15 μL of 4× [concentration] of potentiator. The second 2^{nd} addition is 15 μL of 4× [concentration] of agonist or challenge. This achieves 1× concentration of all compounds only after 2^{nd} addition. The 1^{st} and 2^{nd} additions are performed separately using the FLIPR, which give 2 different data files. Compounds are pretreated at least 30 minutes before agonist addition.

Results are analyzed by dividing the peak fluorescent value of the FLIPR response by the time point after agonist addition to achieve a ratio response. The ratios are then analyzed by curve fitting programs. Since potent compounds can give an inverted U dose response curve (due to effects on endogenous glutamate by the potentiators) points are deleted at concentrations higher than the concentration that gives the maximum effect. Maximum values for dose response curves (forced fitting) are derived from standards on the plate.

Compound Preparation and Glutamate Challenge:

Compounds are delivered as 10 mM DMSO stocks or as powders. Powders are solubilized in DMSO at 10 mM (as solubility allows). Compounds are sonicated in a heated water bath (35-40° C.) for at least 20 minutes. Compounds are then added to assay drug buffer as 40 μL top [concentration] (4× the 10 uM top screening concentration).

In order to test compounds against an EC10 to EC20 concentration of glutamate, multiple glutamate challenge plates for the 2^{nd} FLIPR addition are prepared. The best challenge for a particular assay is determined by examining the glutamate dose response and 1-4 test plates.

EC_{5 0} values of the compounds of the invention are preferably 10 micromolar or less, more preferably 1 micromolar or less, even more preferably 100 nanomolar or less.

When introducing elements of the present invention or the exemplary embodiments) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. A compound of formula I, or a pharmaceutically acceptable salt thereof, 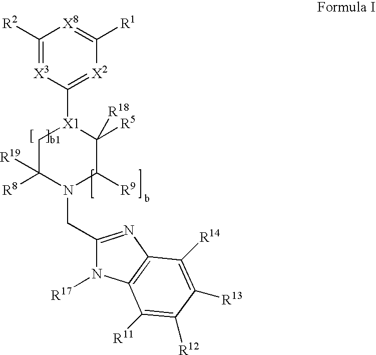
wherein:
X³=CR⁶;
X²=CR⁴;
X⁸=CR³;
R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ wherein each of R¹, R², R³, R⁴ and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, C(O)NR¹⁰¹R¹⁰², NR¹⁰¹C(O)R¹⁰³, and C(O)R¹⁰³;
or two substituents bonded to adjacent carbon atoms of the ring containing X², X³ and X⁸, together with the adjacent carbon atoms, form a heterocyclic or carbocyclic ring which is optionally substituted with one or more R¹⁰, wherein each R¹⁰ is independently selected from the group consisting of hydrogen, —CN, halogen, —C(O)R¹⁰¹, —C(O)NR¹⁰¹R¹⁰², —NR¹⁰¹R¹⁰², —OR¹⁰¹, or —R¹⁰¹;
q is 0, 1 or 2;
each R¹⁰¹ and each R¹⁰² is independently selected from the group consisting of hydrogen, alkyl, alkenyl, alkynyl, cycloalkyl, aryl, heterocycloalkyl and heteroaryl;
wherein each R¹⁰¹ and R¹⁰² alkyl, alkenyl alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl,
R¹⁰³ is independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
X¹=CR⁷;
b=0, 1 or 2;
b1=1 or 2;
each of R⁵, R⁸ and R⁹ is independently selected from the group consisting of halogen, cyano, —R⁴⁰¹, —OR⁴⁰¹, —C(O)OR⁴⁰¹ and —NR⁴⁰¹R⁴⁰²;
R⁷ is hydrogen, halogen, hydroxyl, alkyl, alkoxy, cyano or alkyl-CO—;
or R⁵ and R⁷ taken together form a second bond;
R¹⁶ is hydrogen, halogen or alkyl;
R¹⁹ is hydrogen or —R⁸ and —R¹⁹ together form ═O;
wherein R⁴⁰¹ and R⁴⁰² are independently selected from the group consisting of hydrogen, alkyl, alkenyl, cycloalkyl, aryl, heterocycloalkyl and heteroaryl;
wherein each of the R⁴⁰¹ and R⁴⁰² alkyl alkenyl, cycloalkyl, aryl, heterocycloalkyl and heteroaryl substituents is optionally independently substituted with one or more substituents independently selected from the group consisting of halogen, hydroxy, cyano, nitro, —R⁴¹¹, —C(O)R⁴¹³, —C(O)OR⁴¹³, —C(O)NR⁴¹¹R⁴¹², —OR⁴¹¹, —OC(O)R⁴¹³, —NR⁴¹¹R⁴¹², —NR⁴¹¹C(O)R⁴¹³, —NR⁴¹¹C(O)OR⁴¹³, —NR⁴¹¹S(O)₂R⁴¹³, —S(O)ₜR⁴¹³, —S(O)₂NR⁴¹¹R⁴¹²;
t is 0, 1 or 2;
R⁴¹¹ and R⁴¹² are independently selected from the group consisting of hydrogen, alkyl, cycloalkyl, aryl, heterocycloalkyl and heteroaryl,
R⁴¹³ is independently selected from the group consisting of alkyl, cycloalkyl, aryl, heterocycloalkyl and heteroaryl;
wherein the R⁴¹¹, R⁴¹² and R⁴¹³ alkyl, cycloalkyl, aryl, heterocycloalkyl and heteroaryl substituents are each optionally independently substituted with one or more substituents independently selected from the group consisting of halogen, hydroxy, cyano, nitro, alkyl, aryl, heterocycloalkyl, heteroaryl, haloalkyl, hydroxyalkyl, carboxy, alkoxy and alkoxycarbonyl;
or R⁴ and R⁵ together with the atoms connecting R⁴ and R⁵ form a 5-7-membered carbocyclic or heterocyclic ring optionally containing a heteroatom selected from O, N and S;
or if b=1 and b1=1, R⁵ and R⁹ together with the atoms connecting R⁵ and R⁹ form a 5-7-membered carbocyclic or heterocyclic ring containing up to two heteroatoms selected from O, N and S, wherein the carbocyclic or heterocyclic ring is optionally substituted with one or more substitutents selected from halogen, cyano, alkyl, cycloalkyl, heterocycloalkyl, aryl, heteroaryl or C(O)R²⁰, wherein R²⁰ is alkyl, cycloalkyl, heterocycloalkyl, aryl or heteroaryl and R²⁰ is optionally substituted with one or more substituents independently selected from the group consisting of alkyl, alkoxy, aryloxy, cyano, —CO₂-alkyl, and OC(O)alkyl;
or R⁴ and R⁷ together with the atoms connecting R⁶ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms R⁴ and R⁷ contains a heteroatom selected from the group of O, N and S;
or R⁵ and R⁷ together with the atoms connecting R⁵ and R⁷ form a 3-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;
wherein the carbocyclic or heterocyclic ring formed by R⁴ and R⁷ together with the atoms connecting R⁴ and R⁷, or by R⁵ and R⁷ together with the atoms connecting R⁵ and R⁷ is optionally substituted with one or more substitutents independently selected from halogen, cyano, alkyl, cycloalkyl, heterocycloalkyl, aryl, heteroaryl and —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl or —OC(O)alkyl;
R¹⁷ is selected from the group consisting of alkyl, alkenyl, cycloalkyl, and cycloalkenyl, wherein the R¹⁷ alkyl, alkenyl, cycloalkyl, or cycloalkenyl is optionally substituted with one or more substituents independently selected from the group consisting of halogen, hydroxy, cyano, nitro, —R⁵⁰¹, —OR⁵⁰¹, —NR⁵⁰¹R⁵⁰², —S(O)ᵥR⁵⁰³, —S(O)₂NR⁵⁰¹R⁵⁰², —NR⁵⁰¹S(O)₂R⁵⁰³, —OC(O)R⁵⁰³, —C(O)OR⁵⁰³, —C(O)NR⁵⁰¹R⁵⁰², —NR⁵⁰¹C(O)R⁵⁰³, and —C(O)R⁵⁰³;
v is 0, 1 or 2;
wherein each R⁵⁰¹ and each R⁵⁰² is independently selected from the group consisting of hydrogen, alkyl, alkenyl, cycloalkyl, cycloalkenyl, aryl, heterocycloalkyl and heteroaryl;
R¹¹, R¹², R¹³ and R¹⁴ are each independently selected from the group consisting of halogen, cyano, —R⁶⁰¹, —C(O)OR⁶⁰¹, —C(O)NR⁶⁰¹R⁶⁰², —OR⁶⁰¹, —OC(O)R⁶⁰², —NR⁶⁰¹R⁶⁰², and —NR⁶⁰¹C(O)R⁶⁰²
wherein R⁶⁰¹ and R⁶⁰² are each independently selected from the group consisting of hydrogen, alkyl, alkenyl, cycloalkyl, aryl, heterocycloalkyl and heteroaryl;
wherein the R⁶⁰¹ and R⁶⁰² alkyl, alkenyl, cycloalkyl, aryl, heterocycloalkyl and heteroaryl substituents are each optionally independently substituted with one or more substituents independently selected from the group consisting of halogen, hydroxy, cyano, nitro, —R⁶¹¹, —C(O)R⁶¹³, —C(O)OR⁶¹³, —C(O)NR⁶¹¹R⁶¹², —OR⁶¹¹, —OC(O) R⁶¹³, —NR⁶¹¹R⁶¹², —NR⁶¹¹C(O)R⁶¹³, —NR⁶¹¹C(O)OR⁶¹³, —NR⁶¹¹S(O)₂R⁶¹³, —S(O)ᵢR⁶¹³, —S(O)₂NR⁶¹¹R⁶¹²;
u is 0, 1 or 2;
R⁶¹¹ and R⁶¹² are each independently selected from the group consisting of hydrogen, alkyl, cycloalkyl, aryl, heterocycloalkyl and heteroaryl;
R⁶¹³ is independently selected from the group consisting of alkyl, cycloalkyl, aryl, heterocycloalkyl and heteroaryl.

2. The compound of claim 1, or a pharmaceutically acceptable salt thereof, wherein R¹⁷ is selected from the group consisting of alkyl and cycloalkyl; wherein the R¹⁷ alkyl and cycloalkyl substituents are optionally substituted with one or more substituents independently selected from the group consisting of halogen, cyano, —OR⁵⁰¹, and —NR⁵⁰¹R⁵⁰².

3. The compound of claim 1, or a pharmaceutically acceptable salt thereof, wherein R⁷ is hydrogen, fluoro or alkyl.

4. The compound of claim 1, or a pharmaceutically acceptable salt thereof, wherein each of R¹¹, R¹², R¹³ and R¹⁴ is independently selected from the group consisting of hydrogen, halogen, cyano, alkyl, alkoxy, cycloalkyl, aryl, heterocycloalkyl and heteroaryl, wherein the R¹¹, R¹², R¹³ and R¹⁴ alkyl, cycloalkyl, aryl, heterocycloalkyl and heteroaryl substituents are optionally independently substituted as in the compound of formula I.

5. The compound of claim 4, or a pharmaceutically acceptable salt thereof, wherein each of R¹¹, R¹², R¹³ and R¹⁴ is independently selected from the group consisting of hydrogen, cyano and halogen.

6. The compound of claim 1, or a pharmaceutically acceptable salt thereof, wherein b=1 and b1=0.

7. The compound of claim 1, or a pharmaceutically acceptable salt thereof, wherein b=1 and b1=1.

8. The compound of claim 1, or a pharmaceutically acceptable salt thereof, wherein the compound of formula I has the formula II 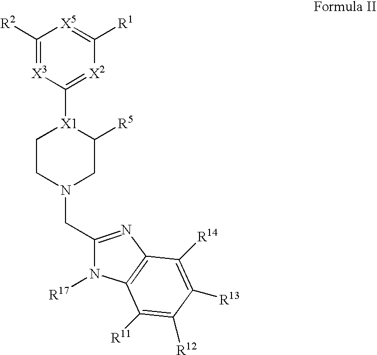
wherein,
R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ or, wherein each of R¹, R², R³, R⁴ and R⁶ alkyl, alkenyl, cycloalkyl, cycloalkenyl, heterocycloalkyl, aryl or heteroaryl is optionally independently substituted with one or more substitutents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, C(O)NR¹⁰¹R¹⁰², NR¹⁰¹C(O)R¹⁰³, —C(O)R¹⁰³;
R⁵ is selected from the group consisting of halogen, —R⁴⁰¹, —OR⁴⁰¹, and —NR⁴⁰¹R⁴⁰²;
R⁷ is hydrogen, halogen, hydroxyl, alkyl, or alkoxy,
or R⁴ and R⁷ together with the atoms connecting R⁴ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms connecting R⁴ and R⁷ contains a heteroatom selected from the group of O, N and S;
or R⁵ and R⁷ together with the atoms connecting R⁵ and R⁷ form a 3-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;
wherein the carbocyclic or heterocyclic ring formed by R⁴ and R⁷ together with the atoms connecting R⁴ and R⁷, or by R⁵ and R⁷ together with the atoms connecting R⁵ and R⁷, is optionally substituted with one or more substitutents independently selected from halogen, cyano, alkyl, cycloalkyl, heterocycloalkyl, aryl, heteroaryl and —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

9. The compound of claim 8, or a pharmaceutically acceptable salt thereof, wherein R⁷ is hydrogen or fluoro.

10. The compound of claim 8, or a pharmaceutically acceptable salt thereof, wherein R⁵ is hydrogen, halogen or alkyl optionally substituted with one or more fluorines.

11. The compound of claim 8, or a pharmaceutically acceptable salt thereof, wherein R¹⁷ is selected from the group consisting of alkyl and cycloalkyl, wherein the R¹⁷ alkyl and cycloalkyl substituent is optionally substituted as in the compound of formula II.

12. The compound of claim 8, or a pharmaceutically acceptable salt thereof, wherein R¹¹, R¹², R¹³ and R¹⁴ are each independently selected from the group consisting of hydrogen, halogen, cyano, alkyl, alkoxy, cycloalkyl, aryl, heterocycloalkyl and heteroaryl, wherein the R¹¹, R¹², R¹³ or R¹⁴ alkyl, cycloalkyl, aryl, heterocycloalkyl and heteroaryl are each optionally independently substituted as in the compound of formula II.

13. The compound of claim 12, or a pharmaceutically acceptable salt thereof, wherein each of R¹¹, R¹², R¹³ and R¹⁴ is independently selected from the group consisting of hydrogen, cyano and halogen.

14. The compound of claim 8, or a pharmaceutically acceptable salt thereof, wherein R⁵ and R⁷ together with the atoms connecting R⁵ and R⁷ form a 5-7-membered carbocyclic or heterocyclic ring that is optionally substituted as in the compound of formula II.

15. The compound of claim 8, or a pharmaceutically acceptable salt thereof, wherein the compound of formula II has the formula III, 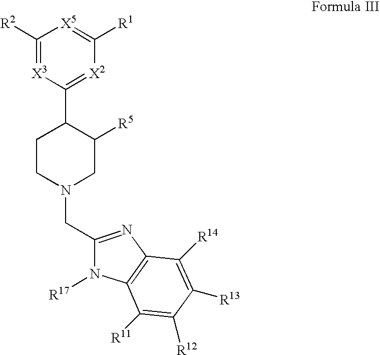
wherein
R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ or, wherein each of R¹, R², R³, R⁴ and R⁶alkyl alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —C(O)NR¹⁰¹R¹⁰², —NR¹⁰¹C(O)R¹⁰³, and —C(O)R¹⁰³; and
R⁵ is hydrogen, halogen or alkyl optionally substituted with one or more fluorines.

16. The compound of claim 15, or a pharmaceutically acceptable salt thereof, wherein R¹⁴ is selected from the group consisting of hydrogen and halogen;
R¹³ is selected from the group consisting of hydrogen, halogen, cyano, alkyl, amino, heterocycloalkyl and heteroaryl;
R¹² is selected from the group consisting of hydrogen, halogen, cyano, alkyl, heterocycloalkyl and heteroaryl; and
R¹¹ is selected from the group consisting of hydrogen, halogen, alkyl, aryl, heterocycloalkyl and heteroaryl.

17. The compound of claim 15, or a pharmaceutically acceptable salt thereof, wherein R⁵ is hydrogen.

18. The compound of claim 15, or a pharmaceutically acceptable salt thereof, wherein R⁵ is alkyl optionally substituted with one or more fluorines.

19. The compound of claim 15, or a pharmaceutically acceptable salt thereof, wherein R⁵ and the aromatic ring containing X², X³ and X⁸ are cis- to each other.

20. The compound of claim 15, or a pharmaceutically acceptable salt thereof, wherein R¹⁷ is alkyl or cycloalkyl, wherein the R¹⁷ alkyl or cycloalkyl substituent is optionally substituted as in the compound of formula III.

21. The compound of claim 15, or a pharmaceutically acceptable salt thereof, wherein R¹⁴ is hydrogen, fluoro or bromo; R¹³ is hydrogen, cyano halogen, methyl or amino; R¹² is selected from the group consisting of hydrogen, bromo, fluoro, cyano, methyl, methoxy and methoxypyridinyl; and R¹¹ is selected from the group consisting of bromo, fluoro, phenyl and methoxypyridinyl.

22. The compound of claim 15, or a pharmaceutically acceptable salt thereof, wherein R¹⁷ is methyl, cyclopropyl, fluoroethyl, fluoromethyl, methoxyethyl or methoxymethyl.

23. The compound of claim 1, or a pharmaceutically acceptable salt thereof, wherein the compound of formula I has the formula IV, 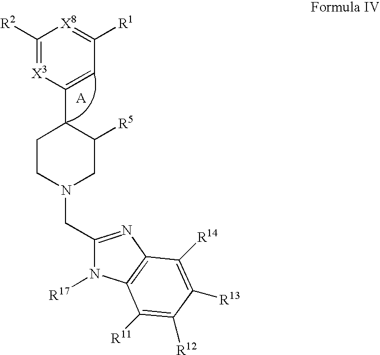
wherein
X³=CR⁶;
X⁸=CR³;
R¹, R², R³, and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, C(O)R¹⁰¹, C(O)NR¹⁰¹R¹⁰², —NR¹⁰¹R¹⁰², or wherein each of R¹, R², R³, and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)_{q}NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, and —C(O)R¹⁰³;
R⁵ is hydrogen, halogen or alkyl; and
wherein ring A is a 5-7-membered carbocyclic or heterocyclic ring, wherein A is optionally substituted with one or more substitutents independently selected from halogen, cyano; alkyl optionally substituted with heterocycloalkyl; cycloalkyl, heterocycloalkyl, aryl, heteroaryl C(O)OR²⁰ or C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, CO₂-alkyl, or OC(O)alkyl.

24. The compound of claim 23, or a pharmaceutically acceptable salt thereof, wherein the compound of formula IV is a compound of formula IVa: 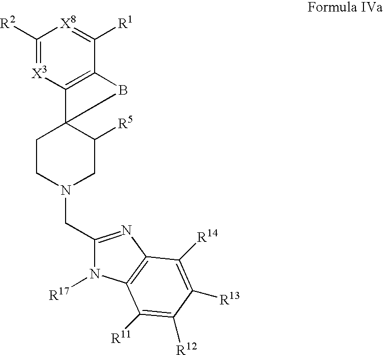
wherein B is a divalent chain selected from the group consisting of ethylene, ethynelene, propylene, butylene, methylenoxy, methylenethioxy, methylenamino, ethylenoxy, ethylenethioxy, and ethylenamino, wherein the carbons or the N of the methylenamino or ethylenamino divalent chain and the carbons of the ethylene, ethynelene, propylene, butylene, metheylenoxy, ethylenoxy, methylenethioxy, and ethylenethioxy divalent chain are each optionally independently substituted with one or more substitutents independently selected from halogen, cyano; alkyl optionally substituted with heterocycloalkyl; cycloakyl, heterocycloalkyl, aryl, heteroaryl or C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, CO₂-alkyl, or OC(O)alkyl.

25. The compound of claim 24, or a pharmaceutically acceptable salt thereof, wherein the N of the methylenamino or ethylenamino is optionally substituted with one or more substitutents independently selected from halogen, cyano, alkyl, cycloalkyl, heterocycloalkyl, aryl, heteroaryl or C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, CO₂-alkyl, or OC(O)alkyl.

26. The compound of claim 1, or a pharmaceutically acceptable salt thereof, wherein 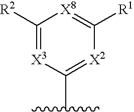
is selected from the group consisting of
4-fluoro-2-methoxyphenyl, 5-fluoro-2-methoxyphenyl, 5-chloro-2-methoxyphenyl, 5-chloro-2-ethoxyphenyl, 5-chloro-2-propoxyphenyl, 5-chloro-2-isobutoxyphenyl, isobutoxyphenyl, butoxyphenyl, 5-Chloro-2-((S)-2-methyl-butoxy)-phenyl, 5-Chloro-2-((R)-2-methyl-butoxy)-phenyl, 2-butoxy-5-chlorophenyl, 5-Chloro-2-(tetrahydro-pyran-2-ylmethoxy)phenyl, 5-Chloro-2-(3-methyl-oxetan-3-ylmethoxy-phenyl, 5-Chloro-2-(tetrahydro-furan-2-ylmethoxy)-phenyl, 5-Chloro-2-(tetrahydro-furan-3-ylmethoxy)-phenyl, 5-Chloro-2-(2-methyl-cyclopropylmethoxy)-phenyl, 5-Chloro-2-(2-cyclopropyl-ethoxy)-phenyl, 5-Chloro-2-cyclobutylmethoxy-phenyl, cyclobutylmethoxy-phenyl, 4-fluoro-3-methoxyphenyl, 2-fluoro-6-methoxyphenyl, difluorophenyl, chlorofluorophenyl, chlorophenyl, bromophenyl, dibromophenyl, fluorophenyl, 2-methoxy-4-trifluoromethylphenyl, trifluoromethylphenyl, [dimethylmorpholin-4-yl]methylphenyl, (2-morpholin-4-yl-ethoxy)-phenyl, methylphenyl, dimethylphenyl, 4-chloro-3-trifluoromethylphenyl, methoxyphenyl, dimethoxyphenyl, hydroxyphenyl, phenyl, fluorophenyl, cyclopentylaminocarbonylphenyl, [N-cyclopropylmethyl]propylaminocarbonylphenyl, [methylpyridynyl]aminocarbonylphenyl, fluorochromanyl, ethylphenyl, t-butylphenyl, cyanophenyl, trifluoromethoxyphenyl, isopropoxy phenyl, 2-methoxy-4-trifluoromethylphenyl, 2-methoxy-5-trifluoromethylphenyl, 2-fluoro-5-trifluoromethylphenyl, 2-fluoro-4-trifluoromethylphenyl, bis-trifluoromethylphenyl, hydroxyethylphenyl, 4-fluoro-2-methylphenyl, 5-Chloro-2-prop-2-ynyloxy-phenyl, prop-2-ynyloxy-phenyl, naphthalenyl, aminocarbonylnaphthalenyl, (1-phenyl-ethoxy)-phenyl, (Indan-2-yloxy)-phenyl, [(S)-(tetrahydro-furan-3-yl)oxy]phenyl, (tetrahydro-pyran-4-yloxy)-phenyl, ((S)-1-methyl-pyrrolidin-2-ylmethoxy-phenyl, (2-pyridin-2-yl-ethoxy)-phenyl, ((S)-2-methyl-butoxy)-phenyl, cyclopropyl-ethoxyphenyl, pentoxyphenyl, 3-ethoxypropoxyphenyl, 2-ethoxyethoxyphenyl, 2-isopropoxyethoxyphenyl, 3-dimethylaminopropoxyphenyl, cyclopentylmethoxyphenyl, 2-(2,6-Dimethyl-morpholin-4-yl)-ethoxy)-phenyl, (2,6-Dimethyl-morpholin-4-yl)-phenyl, methoxycarbonylphenyl, methylsulfonyamidophenyl, methyl-cyclopropylmethoxyphenyl, propynyloxyphenyl, 5-chloro-2-propynyloxyphenyl, 5-chloro-2-(3-tetrahydrofuranyl)methoxyphenyl, 5-chloro-2-(3-tetrahydropyranyl)methoxyphenyl, 5-chloro-2-(2-tetrahydrofuranyl)methoxyphenyl, 5-chloro-2-(2-tetrahydropyranyl)methoxyphenyl, ethoxyphenyl, N-(5-methyl-1H-pyrazol-3-yl)aminocarbonylphenyl, 3-fluoro-4-trifluoromethyl-phenyl, 2-fluoro-4-trifluoromethoxyphenyl, 2-methyl-4-trifluoromethoxyphenyl, 4-chloro-2-methylphenyl, 4-fluoro-2-methylphenyl, 2-chloro-4-trifluoromethylphenyl, 2-chloro-4-isopropoxyphenyl, 2-fluoro-4-isopropoxyphenyl, 3-fluoro-4-isopropoxyphenyl, 3-chloro-4-isopropoxyphenyl, 3-chloro-4-ethoxyphenyl, 4-methoxy-2-trifluoromethylphenyl, difluoromethoxyphenyl, 2-fluoro-4-difluoromethoxyphenyl, 2-chloro-4-difluoromethoxyphenyl, trifluorophenyl, tetralinyl, 4-fluoro-2-isopropoxyphenyl, 4-fluoro-3-trifluoromethylphenyl, (2,3-dihydro-1-benzofuran-5-yl), 4-fluoro-2-trifluoromethylphenyl, 4-chloro-2-trifluoromethylphenyl, 2-chloro-4-methylphenyl, 3-chloro-4-trifluoromethoxyphenyl, 2-chloro-4-trifluoromethoxy-phenyl, 2-methoxy-4-trifluoromethoxyphenyl, 2-trifluoromethyl-4-isopropoxyphenyl, 2-fluoro-6-trifluoromethylphenyl, dichlorophenyl, 3-chloro-4-trifluoromethylphenyl, 2-methyl-4-trifluoromethylphenyl, 3-methyl-4-trifluoromethylphenyl, 4-fluoro-2-difluoromethoxyphenyl 3-methoxy-4-trifluoromethylphenyl and positional isomers thereof.

27. The compound of claim 1, or a pharmaceutically acceptable salt thereof, wherein 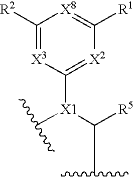
has the structure

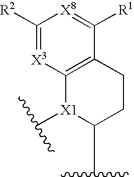

28. The compound of claim 1 or a pharmaceutically acceptable salt thereof, wherein R¹⁷ is selected from the group consisting of cycloalkyl and alkyl optionally substituted with halogen or with alkoxy.

29. The compound of claim 1, or a pharmaceutically acceptable salt thereof, wherein R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring 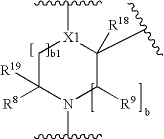
are cis-fused.

30. The compound of claim 1, or a pharmaceutically acceptable salt thereof, wherein R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring 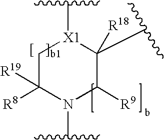
are trans-fused.

31. The compound of claim 1, or a pharmaceutically acceptable salt thereof, wherein the compound is an optically active compound of the formula 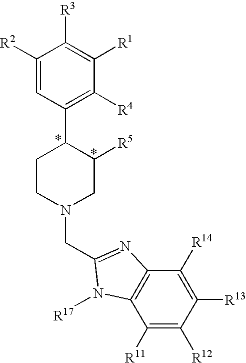
wherein R¹⁷, R¹¹, R¹², R¹³, and R¹⁴ are as defined in formula I; R¹ and R² are each independently halogen or hydrogen; R³ is halogen, hydrogen, alkyl optionally substituted with halogen, or alkoxy optionally substituted with halogen; R⁴ is halogen, hydrogen, alkyl optionally substituted with halogen, or alkoxy, and R⁵ is alkyl optionally substituted with aryloxy, wherein each of the carbons marked with an asterisk independently has the (R) configuration or the (S) configuration, provided that the R⁵ group and the phenyl group substituted with R¹, R², R³, and R⁴ are cis to each other.

32. The compound of claim 1, or a pharmaceutically acceptable salt thereof, wherein the compound is an optically active compound of the formula 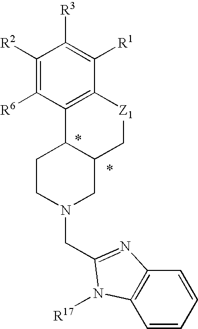
wherein R¹⁷ is as defined in formula I; Z₁ is O or CH₂; R¹ and R² are each independently halogen, hydrogen, or OR¹⁰¹ wherein R¹⁰¹ is alkyl or cycloalkyl, R³ is halogen, hydrogen, alkyl optionally substituted with halogen, or alkoxy optionally substituted with halogen; R⁶ is halogen, hydrogen, alkyl optionally substituted with halogen, or alkoxy; wherein each of the carbons marked with an asterisk independently has the (R) configuration or the (S) configuration.

33. A compound selected from the group consisting of the compounds disclosed in Table 7 herein, or a pharmaceutically acceptable salt thereof.

34. A method for the treatment or prevention of a condition selected from the group consisting of cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder in a mammal, comprising administering a compound of claim 1, or a pharmaceutically acceptable salt thereof, to the mammal.

35. The method of claim 34, wherein the condition is anxiety selected from the group consisting of generalized anxiety disorder, social anxiety disorder, panic disorder, post-traumatic stress disorder and obsessive compulsive disorder.

36. The method of claim 34, wherein the condition is a mood disorder selected from the group consisting of depression, mania, and bipolar disorders.

37. The method of claim 34, wherein the condition is pain selected from the group consisting of acute and chronic pain states, severe pain, intractable pain, neuropathic pain, and post-traumatic pain.

38. A method for treating or preventing neurological and psychiatric disorders associated with glutamate dysfunction, comprising administering to a patient in need thereof an amount of a compound of claim 1, or a pharmaceutically acceptable salt thereof, effective in treating such disorders.

39. The method of claim 38, wherein further comprising administering a metabotropic glutamate receptor agonist.

40. A pharmaceutical composition comprising a compound of claim 1, or a pharmaceutically acceptable salt thereof, and a pharmaceutically acceptable carrier.

41. A composition for treating or preventing a condition selected from the group consisting of cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder in a mammal, wherein the composition contains an amount of the compound of claim 1, or a pharmaceutically acceptable salt thereof, that is effective in the treatment or prevention of such conditions.

42. The composition of claim 41, further comprising a metabotropic glutamate receptor agonist.
